(19) United States
(12) Patent Application Publication
(10) Pub. No.: US 2011/0038854 A1
(43) Pub. Date: Feb. 17, 2011

(54) STRATEGY FOR HOMO- OR HETERO-DIMERIZATION OF VARIOUS PROTEINS IN SOLUTION AND IN CELL

(75) Inventor: Sergei V. Kotenko, East Brunswick, NJ (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(73) Assignee: University of Medicine and Dentistry of New Jersey, Somerset, NJ (US)

(21) Appl. No.: 12/295,619

(22) PCT Filed: Mar. 30, 2007
(86) PCT No.: PCT/US07/65728

§ 371 Date: Oct. 12, 2010

Related U.S. Application Data
(60) Provisional application No. 60/787,407, filed on Mar. 30, 2006.

(51) Int. Cl. A61K 39/395; C07K 14/00; C12N 5/10; C07H 21/00; A61K 38/16; C12N 15/63; C07K 16/00; C12N 5/071; A61P 43/00

(52) U.S. Cl. 424/133.1; 530/350; 435/325; 536/23.1; 514/21.2; 435/320.1; 530/387.3

(57) ABSTRACT

The present invention describes a chimeric polypeptide comprising a first portion comprising a receptor domain, wherein the receptor domain comprises an intracellular region and a transmembrane region; and a second portion comprising a dimerization domain. The present invention also describes a chimeric polypeptide comprising a first portion comprising a receptor domain, wherein the receptor domain comprises a receptor extracellular region; and a second portion comprising a dimerization domain, wherein the dimerization domain comprises an antibody heavy chain region of a Fab fragment or an antibody light chain region. Polynucleotides encoding the chimeric polypeptides and methods of use of the chimeric polypeptides are also described.

### FIELD OF THE INVENTION

This invention relates to a novel method for the generation of homo- and hetero-dimeric protein complexes which include heterodimeric chimeric soluble receptor complexes and constitutively active ligand-independent homo- or heterodimeric membrane-bound chimeric receptor complexes. These chimeric complexes may be used for the treatment of various diseases or as a research tool.

### BACKGROUND OF THE INVENTION

Development of cancer is a multi-step process. It is likely to be started with one point mutation which gives a single cell a minor advantage—either increased rate of uncontrolled proliferation, or unresponsiveness to external signals commanding the cell to commence apoptosis. The immune system also reacts to and destroys the mutated cell and its progeny. However, in rare events, one or more of the cell's offspring, in addition to the initial advantageous mutation, gain other mutations to protect themselves from the immune surveillance. Thus, the immune system is engaged in all steps of tumor development and progression, and the failure of the immune system to recognize and eliminate cancerous cells is a must for tumor survival and progression.

Cytokines regulate a broad array of cellular and immunological functions. They exert their biological activities through the binding to cell surface receptors leading to induction of specific signal transduction events. Cytokines are powerful weapons which are utilized by both cancer cells and the immune system for their own advantages. For example, tumors can benefit from immunosuppressive properties of IL-10. By expressing IL-10, tumors can downregulate antigen presentation and inhibit immune response directed against tumor cells. In addition, several cytokines can stimulate proliferation of tumor cells. If such cytokines are produced by tumors, they may serve as autocrine growth factors for tumor cells. On the other hand, the immune system can inhibit tumor growth through the expression of IFNs, cytokines with strong antiproliferative activity. In addition, IFNs and other cytokines can mobilize and activate various aspects of the immune system to fight cancer.

Many drugs and methods targeting tumors for destruction are being developed and are in trials for their effectiveness. However, it is unlikely that a single drug equally effective in fighting different cancers can ultimately be created, particularly since even a single tumor often represents a pool of cells which have accumulated different multiple mutations. The immune system, on the other hand, is the army of “universal soldiers” which ideally should be able to deal with any type of cancer. In reality, however, the immune system, in the case of cancers, is not able to handle them on its own and several strategies have been developed to help the immune system combat the disease. Such strategies include: the enhancement of the immune system with the use of immunostimulating cytokines, antibodies (Ab) specific for cancer cells (tumor antigens), Ab-cytokine fusion proteins to attract the immune cells to and/or stimulate them at the cancer site, and the boosting of the immune system with in-vitro stimulated immune cells or in-vitro modified cancer cells. The present invention is directed, in part, to the latter strategy and has several advantages including a new method of modifying cancer cells to make them highly immunostimulatory.

In one possible approach to treating cancer using the methods and compositions of the present invention, solid tumors can be transfected with and forced to express constitutively active chimeric IFN receptors (tumor cells can be transfected with expression plasmids or infected with adenovirus harboring the receptor). Infected tumor cells can become highly immunogenic and stimulate an anti-tumor immune response. After stimulating an anti-tumor immune response these modified tumor cells will undergo apoptosis.

This approach will also work for blood cell tumors. Malignant cells can be collected, modified as described for solid tumors and delivered back to the patient. When a primary solid tumor is removed, tumor cells are modified as described above and delivered back to the patient in the case of metastasis or to prevent metastasis. Importantly, targeted delivery of modified tumor cells to sites of residual tumors or metastases can be achieved by using an Ab that is specific for tumor antigens. The extracellular part of the Ab-receptor fusion complex, or the Ab part which is expressed on the cell surface can be specific for tumor antigens. Therefore, modified tumor cells injected into the blood stream may be able to accumulate in tumor sites. This will provide recruitment of immune cells and activation of an antitumor immune response specifically in sites of residual tumors or metastases.

In addition, the present invention can be used to constitutively express active IFN receptors in pathogen-infected cells to stimulate intracellular anti-viral or anti-bacterial protection, and/or make pathogen-infected cells more immunogenic, and/or stimulate apoptosis of infected cells.

### SUMMARY OF THE INVENTION

In one aspect the present invention is directed to a chimeric polypeptide comprising a first portion comprising a receptor domain, wherein the receptor domain comprises an intracellular region and a transmembrane region; and a second portion comprising a dimerization domain. In certain embodiments the first portion consists of the receptor domain and the second portion consists of the dimerization domain. In another embodiment the chimeric polypeptide comprises a linker sequence between the first portion and the second portion.

In another embodiment, the dimerization domain comprises an amino acid sequence derived from an antibody. In certain embodiments the dimerization domain comprises an antibody Fc region or a fragment thereof, an antibody heavy chain region of a Fab region or a fragment thereof, or an antibody light chain region or a fragment thereof. In certain embodiments, the dimerization domain comprises an amino acid sequence selected from the group consisting of SEQ ID NO: 16 to 25.

In another embodiment, the receptor domain comprises an amino acid sequence of a receptor selected from the group consisting of an interleukin receptor, a cytokine receptor, an interferon receptor and a growth factor receptor. In certain embodiments, the receptor domain comprises an amino acid sequence selected from the group consisting of SEQ ID NO: 41 to 50.

In another aspect the present invention is directed to a composition comprising a first chimeric polypeptide wherein the first a chimeric polypeptide comprises a first portion comprising a receptor domain, wherein the receptor domain comprises an intracellular region and a transmembrane region; and a second portion comprising a dimerization domain; and a second chimeric polypeptide wherein the second chimeric polypeptide comprises a first portion comprising a receptor domain, wherein the receptor domain comprises an intracellular region and a transmembrane region; and a second portion comprising a dimerization domain; receptor domain of the second chimeric polypeptide is derived from a receptor the same as the receptor domain of the first chimeric polypeptide; wherein the dimerization domain of the first chimeric polypeptide binds to the dimerization domain of the second chimeric polypeptide resulting in the receptor domain of the first chimeric polypeptide associating with the receptor domain of the second chimeric polypeptide.

In another aspect the present invention is directed to a composition comprising a first chimeric polypeptide wherein the first a chimeric polypeptide comprises a first portion comprising a receptor domain, wherein the receptor domain comprises an intracellular region and a transmembrane region; and a second portion comprising a dimerization domain; and a second chimeric polypeptide wherein the second chimeric polypeptide comprises a first portion comprising a receptor domain, wherein the receptor domain comprises an intracellular region and a transmembrane region; and a second portion comprising a dimerization domain; receptor domain of the second chimeric polypeptide is derived from a receptor different from the receptor domain of the first chimeric polypeptide; wherein the dimerization domain of the first chimeric polypeptide binds to the dimerization domain of the second chimeric polypeptide resulting in the receptor domain of the first chimeric polypeptide associating with the receptor domain of the second chimeric polypeptide.

In another aspect the present invention is directed to a composition comprising a first chimeric polypeptide wherein the first chimeric polypeptide comprises a first portion comprising a receptor domain, wherein the receptor domain comprises an intracellular region and a transmembrane region; and a second portion comprising a dimerization domain comprising an antibody Fc region or a fragment thereof; and a second chimeric polypeptide wherein the second chimeric polypeptide comprises a first portion comprising a receptor domain, wherein the receptor domain comprises an intracellular region and a transmembrane region; and a second portion comprising a dimerization domain comprising an antibody Fc region or a fragment thereof, wherein the receptor domain of the second chimeric polypeptide is derived from a receptor the same as the receptor domain of the first chimeric polypeptide; wherein the dimerization domain of the first chimeric polypeptide binds to the dimerization domain of the second chimeric polypeptide resulting in the receptor domain of the first chimeric polypeptide associating with the receptor domain of the second chimeric polypeptide.

In another aspect the present invention is directed to composition comprising a first chimeric polypeptide wherein the first chimeric polypeptide comprises a first portion comprising a receptor domain, wherein the receptor domain comprises an intracellular region and a transmembrane region; and a second portion comprising a dimerization domain comprising an antibody heavy chain region of a Fab region or a fragment thereof; and a second chimeric polypeptide wherein the second chimeric polypeptide comprises a first portion comprising a receptor domain, wherein the receptor domain comprises an intracellular region and a transmembrane region; and a second portion comprising a dimerization domain comprising an antibody light chain region or a fragment thereof, wherein the receptor domain of the second chimeric polypeptide is derived from a receptor different from the receptor domain of the first chimeric polypeptide; wherein the dimerization domain of the first chimeric polypeptide binds to the dimerization domain of the second chimeric polypeptide resulting in the receptor domain of the first chimeric polypeptide associating with the receptor domain of the second chimeric polypeptide.

In another aspect the present invention is directed to a method of activating a signaling pathway in a cell comprising administering to the cell a composition of the present invention. In one embodiment the signaling pathway is a pathway activated by a receptor selected from the group consisting of an interleukin receptor, a cytokine receptor, an interferon receptor and a growth factor receptor. In another embodiment the signaling pathway is a JAK/STAT signaling pathway or a MAP kinase signaling pathway.

In another aspect the present invention is directed to a method of preventing, treating, or ameliorating a disease related to an increased or extended signaling pathway induced by cytokines or growth factors in a subject comprising administering to the subject a composition according of the present invention wherein the composition activates a signaling pathway that counterbalances, suppresses or alters the increased or extended signaling pathway in the subject. In one embodiment the disease is selected from the group consisting of cancer, malignant conditions, chronic infections with various pathogens such as viruses and bacteria, chronic inflammatory conditions and autoimmune diseases. For example, the Type I, II, and III interferons have antiproliferative/pro-apoptotic activity. Membrane-bound chimeric polypeptides comprising the intracellular domains of the receptors for these interferons would be useful to treat proliferative disorders such as cancer. Proliferation and cell survival can be induced by various ligands including EGF, IL-20, and IL-22 or oncoproteins such as v-Abl or v-Src. Soluble chimeric polypeptides (described in more detail below) comprising the extracellular domains of the receptors for these ligands would be useful to treat proliferative disorders such as cancer.

Similarly, IL-10 has an anti-inflammatory activity. Membrane-bound chimeric polypeptides comprising the intracellular domains of the IL-10 receptor would be useful to treat chronic inflammatory conditions. Conversely, IL-12; IL-23; IFN-gamma (type II IFN), IL-17, IL-22 have proinflammatory activity. Soluble chimeric polypeptides (described in more detail below) comprising the extracellular domains of the receptors for these ligand would be useful to treat chronic inflammatory conditions.

In another aspect the present invention is directed to a method of preventing, treating, or ameliorating a disease related to increased or extended STAT3 and/or STAT5 activity in a subject comprising administering to the subject a composition according to the present invention wherein the composition activates a STAT1 signaling pathway in the subject.

In another aspect the present invention is directed to a chimeric polypeptide comprising a first portion comprising a receptor domain, wherein the receptor domain comprises a receptor extracellular region; and a second portion comprising a dimerization domain, wherein the dimerization domain comprises an antibody heavy chain region of a Fab fragment or an antibody light chain region. In one embodiment, the first portion consists of the receptor domain and the second portion consists of the dimerization domain. In another embodiment, the chimeric polypeptide comprises a linker sequence between the first portion and the second portion. In another embodiment, the receptor domain comprises amino acid sequence from a receptor selected from the group consisting of an interleukin receptor, a cytokine receptor, an interferon receptor and a growth factor receptor. In another embodiment, the receptor domain comprises an amino acid sequence selected from the group consisting of SEQ ID NO: 51 to 55. In another embodiment, the dimerization domain comprises an amino acid sequence selected from the group consisting of SEQ ID NO: 26 to 30.

In another aspect the present invention is direct to a composition comprising first chimeric polypeptide wherein the first chimeric polypeptide is a chimeric polypeptide comprising a first portion comprising a receptor domain, wherein the receptor domain comprises a receptor extracellular region; and a second portion comprising a dimerization domain, wherein the dimerization domain comprises an antibody heavy chain region of a Fab fragment or an antibody light chain region; and a second chimeric polypeptide wherein the second chimeric polypeptide is a chimeric polypeptide comprising a first portion comprising a receptor domain, wherein the receptor domain comprises a receptor extracellular region; and a second portion comprising a dimerization domain, wherein the dimerization domain comprises an antibody heavy chain region of a Fab fragment or an antibody light chain region, wherein the receptor domain of the second chimeric polypeptide is derived from a receptor different from the receptor domain of the first chimeric polypeptide; wherein the dimerization domain of the first chimeric polypeptide binds to the dimerization domain of the second chimeric polypeptide resulting in the receptor domain of the first chimeric polypeptide associating with the receptor domain of the second chimeric polypeptide.

In another aspect the present invention is direct to a method of inhibiting or reducing activation of a signaling pathway in a cell comprising administering to the cell a composition comprising first chimeric polypeptide wherein the first chimeric polypeptide is a chimeric polypeptide comprising a first portion comprising a receptor domain, wherein the receptor domain comprises a receptor extracellular region; and a second portion comprising a dimerization domain, wherein the dimerization domain comprises an antibody heavy chain region of a Fab fragment or an antibody light chain region; and a second chimeric polypeptide wherein the second chimeric polypeptide is a chimeric polypeptide comprising a first portion comprising a receptor domain, wherein the receptor domain comprises a receptor extracellular region; and a second portion comprising a dimerization domain, wherein the dimerization domain comprises an antibody heavy chain region of a Fab fragment or an antibody light chain region, wherein the receptor domain of the second chimeric polypeptide is derived from a receptor different from the receptor domain of the first chimeric polypeptide; wherein the dimerization domain of the first chimeric polypeptide binds to the dimerization domain of the second chimeric polypeptide resulting in the receptor domain of the first chimeric polypeptide associating with the receptor domain of the second chimeric polypeptide. In one embodiment the signaling pathway is activated by a cytokine. In another embodiment the cytokine is selected from the group consisting of type I IFN, type II IFN, type III IFN, IL-10, IL-19, IL-20, IL-22, IL-24, IL-26 and Epidermal Growth Factor (EGF)

In another aspect the present invention is directed to a polynucleotide encoding a chimeric polypeptide according to the present invention. In one embodiment the polynucleotide encodes a receptor domain comprising an amino acid sequence selected from the group consisting of SEQ ID NO: 41 to 50 or a receptor domain comprising an amino acid sequence selected from the group consisting of SEQ ID NO: 51 to 55. In another embodiment the polynucleotide encodes a dimerization domain comprising an amino acid sequence selected from the group consisting of SEQ ID NO: 16 to 25 or a dimerization domain comprising an amino acid sequence selected from the group consisting of SEQ ID NO: 26 to 30. In another embodiment the polynucleotide the polynucleotide comprises a sequence selected from the group consisting of SEQ ID NO: 86 to 95 and 111 to 120 or a sequence selected from the group consisting of SEQ ID NO: 96 to 100 and 121 to 125.

In another aspect the present invention is directed to a vector comprising a polynucleotide sequence of the present invention. In one embodiment the vector comprises a polynucleotide wherein the polynucleotide sequence is operably linked to a promoter selected from the group consisting of HSV, TK, RSV, SV40, CMV, elongation factor 1α (EF-1α), and β-actin promoters.

In another aspect the present invention is directed to a cell comprising a chimeric polypeptide, a polynucleotide or a vector of the present invention.

In another aspect the present invention is directed to a composition comprising a chimeric polypeptide, a polynucleotide or a vector of the present invention.

### BRIEF DESCRIPTION OF THE DRAWINGS

FIG. 1. Schematic models of various natural and fusion protein complexes.

FIG. 2. Interleukins and Growth Hormones as Oncoproteins.

FIG. 3. Role of Cytokines in Cancer.

FIG. 4. Class II cytokine receptor family and its associated cytokines.

FIG. 5. Ligand-independent constitutively active IFN-γ signaling.

FIG. 6. Balancing STAT1 and STAT3 Activation.

FIG. 7. IL-10R-Ab chimeric complex neutralizes IL-10 signaling.

FIG. 8. Nucleotide and amino acid sequences of chimeric antibody-receptor constructs.

FIG. 9. Schematic of relative position of primers used to amplify various parts of Ab molecules.

FIG. 10. Constitutive ligand-independent STAT activation of receptors transiently expressed in COS cells.

### DETAILED DESCRIPTION OF THE INVENTION

In one aspect the present invention is directed to a chimeric polypeptide comprising a first portion comprising a receptor domain, wherein the receptor domain comprises an intracellular region and a transmembrane region; and a second portion comprising a dimerization domain.

A chimeric polypeptide is a human-engineered or in vivo mutated protein that is encoded by a nucleotide sequence made by a splicing together of two or more complete or partial genes or cDNA. The pieces used may be from different species.

A receptor is a protein on the cell membrane or within the cytoplasm or cell nucleus that binds to a specific molecule (a ligand). A receptor domain is a portion or fragment of a receptor amino acid sequence or an amino acid sequence derived from the amino acid sequence of a receptor. Different receptor domains can be characterized based on where they are located in relation to a cellular membrane. Receptors generally have an intracellular region, a transmembrane region, and an extracellular region. The transmembrane region of a receptor is that portion of a receptor that is embedded within the cell membrane. The transmembrane region is generally a hydrophobic region. The intracellular region is in the cytoplasm of the cell. This region interacts with cytoplasmic molecules and activates signaling pathways. As used herein, the term “intracellular domain” means the intracellular domain of the receptor or a portion of the intracellular domain that is necessary for activation of the receptor signaling pathways. The extracellular region is outside the cell. This region interacts with the receptor ligands. As used herein, the term “extracellular domain” means the extracellular domain of the receptor or a portion of the extracellular domain that is necessary for binding to its ligand.

A dimerization domain is an amino acid sequence capable of associating with or binding to another dimerization domain. The association or binding may be covalent or non-covalent. The association or binding may be facilitated by the binding of non-amino acid molecules to the dimerization domain. For example, an avidin molecule attached to one dimerization domain and a biotin molecule attached to another dimerization domain would result in formation of an avidin-biotin bridge, thus promoting dimerization of the two domains. In another embodiment, the association or binding is through protein-protein interactions of the dimerization domains, for example, two leucine zipper domains. In one embodiment the dimerization domain is derived from an antibody.

In certain embodiments the first portion consists of the receptor domain and the second portion consists of the dimerization domain. In another embodiment the chimeric polypeptide comprises a linker sequence between the first portion and the second portion. A linker is a molecule that acts as a bridge between different domains of the chimeric polypeptide. A linker sequence is a polypeptide sequence that acts as a bridge. Linker sequences can be of any length. In one embodiment the linker sequence is less than 50 amino acids. In other embodiments the linker sequence is less than 40, less than 30 or less than 20 amino acids in length. In certain embodiments, the linker sequence is between 21 and 25 amino acids in length. In other embodiments the linker sequence comprises an amino acid sequence comprising a sequence selected from the group consisting of amino acids 221-245 of SEQ ID NO: 11; amino acids 464-470 of SEQ ID NO: 11; amino acids 236-260 of SEQ ID NO: 12; amino acids 479-490 of SEQ ID NO: 12; amino acids 236-260 of SEQ ID NO: 13; amino acids 227-241 of SEQ ID NO: 14; and amino acids 229-253 of SEQ ID NO: 15.

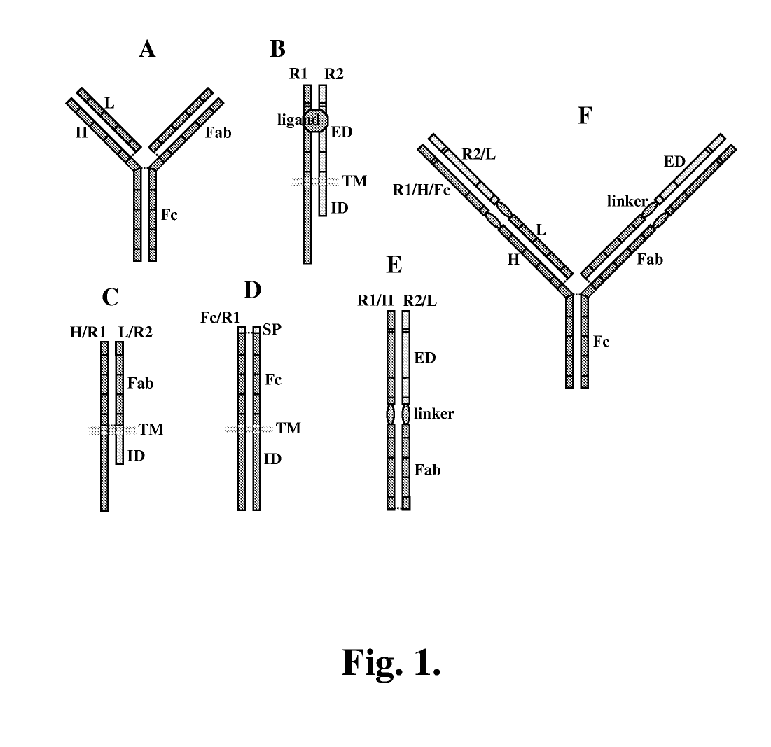

In another embodiment, the dimerization domain comprises an amino acid sequence derived from an antibody. Generally, an antibody (or immunoglobulin) is a large Y-shaped protein used by the immune system to identify and neutralize foreign objects like bacteria and viruses. Each antibody recognizes a specific antigen unique to its target. The basic unit of each antibody is a monomer (one Ig unit). The monomer is a “Y”-shaped molecule that consists of four polypeptide chains; two identical heavy chains and two identical light chains connected by disulfide bonds or through protein-protein interaction (FIG. 1A). The Fc region is derived from the stem of the “Y,” and is composed of two heavy chains that each contribute two to three constant domains (depending on the class of the antibody). Fc regions are capable of binding to Fc regions with identical or similar amino acid sequences. Such a dimer is referred to a homodimer.

Each end of the forked portion of the “Y” on the antibody is called the Fab region (Fragment, antigen binding). It is composed of a portion of the heavy chain and one light chain. These domains shape the antigen binding site at the amino terminal end of the monomer. Fc regions are capable of binding to Fc regions with identical or similar amino acid sequences. The heavy chain of the Fab fragment binds to and forms a dimer with the light chain. Because the heavy and light chains are different amino acid sequences, such a dimer is referred to a heterodimer.

The following tables refer to chimeric polypeptides, and fragments thereof, and polynucleotides that encode the chimeric polypeptides, and fragments thereof, that are disclosed in the Examples below. The present invention is directed to the use of any of these amino acid of polynucleotide sequences to generate a chimeric polypeptide or polynucleotide of the present invention. Accordingly, the present invention is directed to a chimeric polypeptide comprising and a polynucleotide encoding an amino acid sequence selected from the group consisting of SEQ ID NO: 1 to 55, and a polynucleotide selected from the group consisting of SEQ ID NO: 56 to 125.

mH2/γR1

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-484 | 1 | Full Length | 1-1452 | 56 |
| | | | Full Length + Stop | 1-1455 | 71 |
| | | | Codon | | |
| Ab portion | 1-241 | 16 | Ab portion | 1-723 | 86 |
| Trans- | 242-265 | 31 | Trans-membrane | 724-795 | 101 |
| membrane | | | | | |
| Intracellular | 266-484 | 41 | Intracellular | 796-1452 | 111 |

mL2/γR2

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-327 | 2 | Full Length | 1-981 | 57 |
| | | | Full Length + Stop | 1-984 | 72 |
| | | | Codon | | |
| Ab portion | 1-236 | 17 | Ab portion | 1-711 | 87 |
| Trans- | 237-231 | 32 | Trans-membrane | 712-783 | 102 |
| membrane | | | | | |
| Intracellular | 262-327 | 41 | Intracellular | 784-981 | 112 |

SPd6xHis-hH2/γR1

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-20 | 3 | Full Length | 1-1485 | 58 |
| | | | Full Length + Stop | 1-1488 | 73 |
| | | | Codon | | |
| Ab portion | 27-252 | 18 | Ab portion | 79-756 | 88 |
| Trans- | 253-276 | 33 | Trans-membrane | 757-828 | 103 |
| membrane | | | | | |
| Intracellular | 277-495 | 43 | Intracellular | 829-1485 | 113 |

SPFL-hL2/γR2

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-342 | 4 | Full Length | 1-1026 | 59 |
| | | | Full Length + Stop | 1-1029 | 74 |
| | | | Codon | | |
| Ab portion | 28-252 | 19 | Ab portion | 82-756 | 89 |
| Trans- | 253-276 | 34 | Trans-membrane | 757-828 | 104 |
| membrane | | | | | |
| Intracellular | 277-342 | 44 | Intracellular | 829-1026 | 114 |

SPFL-mFc/γR1

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-597 | 5 | Full Length | 1-1491 | 60 |
| | | | Full Length + Stop | 1-1494 | 75 |
| | | | Codon | | |
| Ab portion | 28-254 | 20 | Ab portion | 82-762 | 90 |
| Trans- | 255-278 | 35 | Trans-membrane | 763-834 | 105 |
| membrane | | | | | |
| Intracellular | 279-597 | 45 | Intracellular | 835-1491 | 115 |

SPFL-mFc/IFN-λR1

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-548 | 6 | Full Length | 1-1637 | 61 |
| | | | Full Length + Stop | 1-1640 | 76 |
| | | | Codon | | |
| Ab portion | 30-254 | 21 | Ab portion | 88-762 | 91 |
| Trans- | 255-277 | 36 | Trans-membrane | 763-831 | 106 |
| membrane | | | | | |
| Intracellular | 278-548 | 46 | Intracellular | 832-1637 | 116 |

SPFL-mFc/IFN-αR2

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-526 | 7 | Full Length | 1-1577 | 62 |
| | | | Full Length + Stop | 1-1560 | 77 |
| | | | Codon | | |
| Ab portion | 30-254 | 22 | Ab portion | 88-762 | 92 |
| Trans- | 255-275 | 37 | Trans-membrane | 763-825 | 107 |
| membrane | | | | | |
| Intracellular | 276-526 | 47 | Intracellular | 826-1577 | 117 |

SPFL-mFc/IL-20R1

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-557 | 8 | Full Length | 1-1667 | 63 |
| | | | Full Length + Stop | 1-1670 | 78 |
| | | | Codon | | |
| Ab portion | 30-254 | 23 | Ab portion | 88-762 | 93 |
| Trans- | 255-278 | 38 | Trans-membrane | 763-836 | 108 |
| membrane | | | | | |
| Intracellular | 279-557 | 48 | Intracellular | 837-1667 | 118 |

SPFL-mFc/IL-20R1

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-600 | 9 | Full Length | 1-1800 | 65 |
| | | | Full Length + Stop | 1-1803 | 79 |
| | | | Codon | | |
| Ab portion | 30-254 | 24 | Ab portion | 88-762 | 94 |
| Trans- | 255-277 | 38 | Trans-membrane | 763-831 | 109 |
| membrane | | | | | |
| Intracellular | 278-600 | 49 | Intracellular | 832-1800 | 119 |

SPFL-mFc/EGFR

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-818 | 10 | Full Length | 1-2447 | 65 |
| | | | Full Length + Stop | 1- | 80 |
| | | | Codon | | |
| Ab portion | 30-254 | 25 | Ab portion | 88-762 | 95 |
| Trans- | 255-276 | 40 | Trans-membrane | 763-828 | 110 |
| membrane | | | | | |
| Intracellular | 277-818 | 50 | Intracellular | 829-2447 | 120 |

IL-10R2-2xlinker-L2-linker-FL

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-479 | 11 | Full Length | 1-1437 | 66 |
| | | | Full Length + Stop | 1-1440 | 81 |
| | | | Codon | | |
| Ab portion | 246-463 | 26 | Ab portion | 736-1398 | 96 |
| Extracellular | 1-220 | 51 | Extracellular | 1-660 | 121 |

IL-10R1-2xlinker-H2-linker-6xHis

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-496 | 12 | Full Length | 1-1491 | 67 |
| | | | Full Length + Stop | 1-1494 | 82 |
| | | | Codon | | |
| Ab portion | 261-478 | 27 | Ab portion | 781-1434 | 97 |
| Extracellular | 1-235 | 52 | Extracellular | 1-705 | 122 |

IL-10R1-2xlinker-H2-Fc

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-705 | 13 | Full Length | 1-2115 | 68 |
| | | | Full Length + Stop | 1-2118 | 83 |
| | | | Codon | | |
| Ab portion | 261-705 | 28 | Ab portion | 781-2115 | 98 |
| Extracellular | 1-235 | 53 | Extracellular | 1-705 | 123 |

IFN-λR1-linker-H2-Fc

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-386 | 14 | Full Length | 1-2058 | 69 |
| | | | Full Length + Stop | 1-2061 | 84 |
| | | | Codon | | |
| Ab portion | 242-386 | 29 | Ab portion | 724-2058 | 99 |
| Extracellular | 1-226 | 54 | Extracellular | 1-678 | 124 |

IL-22R1-2xlinker-H2-Fc

| Amino Acid | | | Polynucleotide | | |
|---|---|---|---|---|---|
| | | SEQ ID | | | SEQ ID |
| Description | Residues | NO | Description | Nucleotides | NO |
| Full Length | 1-699 | 15 | Full Length | 1-2097 | 70 |
| | | | Full Length + Stop | 1-2100 | 85 |
| | | | Codon | | |
| Ab portion | 254-699 | 30 | Ab portion | 760-2097 | 100 |
| Extracellular | 1-228 | 55 | Extracellular | 1-684 | 125 |

In certain embodiments the dimerization domain comprises an antibody Fc region or a fragment thereof, an antibody heavy chain region of a Fab region or a fragment thereof, or an antibody light chain region or a fragment thereof. As used herein, the term “Fc region or a fragment thereof” means the Fc region or a fragment thereof that is necessary for homodimer formation. As used herein, the term “an antibody heavy chain region of a Fab region or a fragment thereof” means the antibody heavy chain region of a Fab region or a fragment thereof that is necessary for heterodimer formation with a light chain or a fragment thereof. As used herein, the term “an antibody light chain region or a fragment thereof” means the antibody light chain region or a fragment thereof that is necessary for heterodimer formation with an antibody heavy chain region of a Fab region or a fragment thereof. In certain embodiments, the dimerization domain comprises an amino acid sequence selected from the group consisting of SEQ ID NO: 16 to 25 or an amino acid sequence selected from the group consisting of SEQ ID NO: 26 to 30.

The unique property of antibody molecules can be utilized to design a universal approach of creating various homo- or hetero-dimeric fusion proteins. The overall design is shown in FIG. 1. Generalized structures of natural and designed fusion (or chimeric) protein complexes are schematically presented on FIG. 1. Whole Fc and Fab Ab portions or their fragments that are sufficient for dimerization are utilized to create membrane bound constitutively active receptor complexes. Such receptor complexes do not require a ligand. Fab parts of heavy and light chains of Ab serve as the extracellular domains of the Fab/receptor chimeric complex, heterodimerize on the cell surface without any stimulus and cause heterodimerization of the intracellular domains (ID) of receptor subunits that triggers signal transduction cascade specific for the given receptor subunits. Very often ID of only one receptor subunit determines the specificity of signaling (Kotenko et al., 1996, 1999, 2000, 2001, 2002, 2004). In such cases homodimerization of the R1 type of the receptor subunit is sufficient (Kotenko et al., 1996, 1999, 2000) and Fc/R1 fusion proteins can be utilized to create constitutively active cytokine-specific receptor complex (see FIG. 1 B and C).

In addition, whole Ab, Fab Ab portions or their fragments that are sufficient for dimerization are utilized to create heterodimeric soluble cytokine-specific receptor complexes as schematically shown on FIGS. 1 E and F.

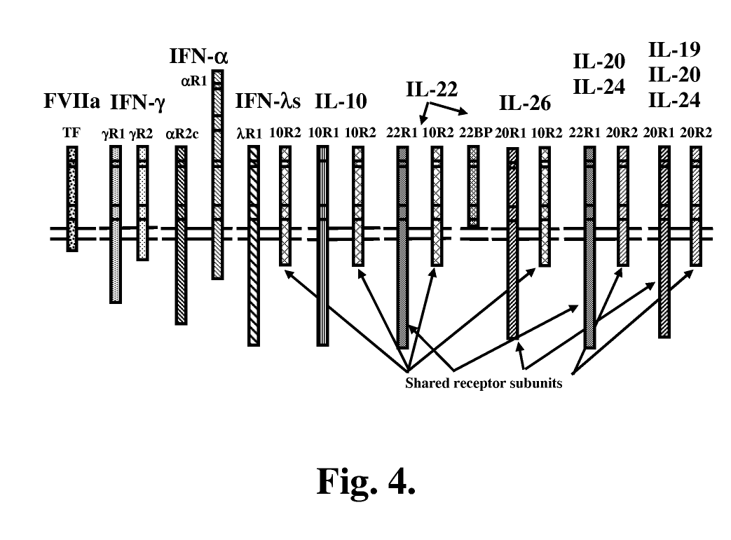

In FIG. 1, R1 can be IFN-αR2c, IFN-γR1, IL-10R1, IL-22R1, IL-20R1 or IFN-λR1; whereas R2 can be IFN-αR1, IFN-γR2, IL-20R2 or IL-10R2. Receptor activation by various IFNs is shown in FIG. 4. The two subunits of the IFN-γ and the type I IFN receptor complexes are indicated, respectively, as “γR1” and “γR2”, and “αR1” and “αR2”. Tissue factor (TF) is a receptor for coagulation factor VIIa (FVIIa). IL-10R2 can be combined with either IL-10R1, IL-22R1, IFN-λR1 or IL-20R1 to assemble the IL-10, IL-22, IFN-λ or IL-26 receptor complexes, respectively. IL-22R1 can also function with IL-20R2, generating the receptor complexes for IL-20 and IL-24. In turn, IL-20R2 can also join IL-20R1 to form the receptor complex for IL-19, IL-20 and IL-24. IL-22BP is the IL-22 binding protein, the only soluble receptor from this family.

In another embodiment, the receptor domain comprises an amino acid sequence of a receptor selected from the group consisting of an interleukin receptor, a cytokine receptor, an interferon receptor and a growth factor receptor. In certain embodiments, the receptor is a receptor shown in FIG. 4. In another embodiment, the receptor is IFNγR, IFNαR, IL-λR, IL-10R, IL-20R, IL-22R or EGFR. In another embodiment, the receptor domain comprises an amino acid sequence selected from the group consisting of SEQ ID NO: 41 to 50.

Further provided by the present invention are isolated polypeptides having an amino acid sequence corresponding to SEQ ID NO.: 1 to 55, or the amino acid sequence of a functionally equivalent fusion protein product, fragment or bioprecursor of the protein. A protein of the invention can be in a substantially purified form, in which case it will generally comprise the polypeptide in a preparation in which more than 90%, e.g. 95%, 98%, or 99% of the polypeptide in the preparation is a polypeptide of the invention. Proteins of the invention can be modified, for example by the addition of histidine residues to assist their purification or by the addition of a signal sequence to promote their secretion from a cell. Proteins having at least 90% sequence identity, for example at least 95%, 98% or 99% sequence identity to the polypeptide protein depicted in SEQ ID NO.: 1 to 55 may be proteins which are amino acid sequence variants, alleles, derivatives, or mutants of the protein depicted in SEQ ID NO.: 1 to 55, and are also provided by the present invention.

In certain embodiments the chimeric polypeptides of the present invention comprise an epitope tag. Common epitopes tags used for this purpose are c-myc, HA, FLAG, V5, and His 6×. In other embodiments the epitope tag sequence comprises an amino acid sequence comprising a sequence selected from the group consisting of amino acids 21-26 of SEQ ID NO: 3; amino acids 22-27 of SEQ ID NOS: 4-10; amino acids 471-478 of SEQ ID NO: 11; and amino acids 491-496 of SEQ ID NO: 12. In certain embodiments a signal peptide is introduced in the beginning of the chimeric polypeptides to target the polypeptide to the membrane.

In another aspect the present invention is directed to a composition comprising a first chimeric polypeptide wherein the first a chimeric polypeptide comprises a first portion comprising a receptor domain, wherein the receptor domain comprises an intracellular region and a transmembrane region; and a second portion comprising a dimerization domain; and a second chimeric polypeptide wherein the second chimeric polypeptide comprises a first portion comprising a receptor domain, wherein the receptor domain comprises an intracellular region and a transmembrane region; and a second portion comprising a dimerization domain; receptor domain of the second chimeric polypeptide is derived from a receptor the same as the receptor domain of the first chimeric polypeptide; wherein the dimerization domain of the first chimeric polypeptide binds to the dimerization domain of the second chimeric polypeptide resulting in the receptor domain of the first chimeric polypeptide associating with the receptor domain of the second chimeric polypeptide.

In another aspect the present invention is directed to a composition comprising a first chimeric polypeptide wherein the first a chimeric polypeptide comprises a first portion comprising a receptor domain, wherein the receptor domain comprises an intracellular region and a transmembrane region; and a second portion comprising a dimerization domain; and a second chimeric polypeptide wherein the second chimeric polypeptide comprises a first portion comprising a receptor domain, wherein the receptor domain comprises an intracellular region and a transmembrane region; and a second portion comprising a dimerization domain; receptor domain of the second chimeric polypeptide is derived from a receptor different from the receptor domain of the first chimeric polypeptide; wherein the dimerization domain of the first chimeric polypeptide binds to the dimerization domain of the second chimeric polypeptide resulting in the receptor domain of the first chimeric polypeptide associating with the receptor domain of the second chimeric polypeptide.

In another aspect the present invention is directed to a composition comprising a first chimeric polypeptide wherein the first chimeric polypeptide comprises a first portion comprising a receptor domain, wherein the receptor domain comprises an intracellular region and a transmembrane region; and a second portion comprising a dimerization domain comprising an antibody Fc region or a fragment thereof; and a second chimeric polypeptide wherein the second chimeric polypeptide comprises a first portion comprising a receptor domain, wherein the receptor domain comprises an intracellular region and a transmembrane region; and a second portion comprising a dimerization domain comprising an antibody Fc region or a fragment thereof, wherein the receptor domain of the second chimeric polypeptide is derived from a receptor the same as the receptor domain of the first chimeric polypeptide; wherein the dimerization domain of the first chimeric polypeptide binds to the dimerization domain of the second chimeric polypeptide resulting in the receptor domain of the first chimeric polypeptide associating with the receptor domain of the second chimeric polypeptide.

In another aspect the present invention is directed to composition comprising a first chimeric polypeptide wherein the first chimeric polypeptide comprises a first portion comprising a receptor domain, wherein the receptor domain comprises an intracellular region and a transmembrane region; and a second portion comprising a dimerization domain comprising an antibody heavy chain region of a Fab region or a fragment thereof; and a second chimeric polypeptide wherein the second chimeric polypeptide comprises a first portion comprising a receptor domain, wherein the receptor domain comprises an intracellular region and a transmembrane region; and a second portion comprising a dimerization domain comprising an antibody light chain region or a fragment thereof, wherein the receptor domain of the second chimeric polypeptide is derived from a receptor different from the receptor domain of the first chimeric polypeptide; wherein the dimerization domain of the first chimeric polypeptide binds to the dimerization domain of the second chimeric polypeptide resulting in the receptor domain of the first chimeric polypeptide associating with the receptor domain of the second chimeric polypeptide.

In another aspect the present invention is directed to a method of activating a signaling pathway in a cell comprising administering to the cell a composition of the present invention. In one embodiment the signaling pathway is a pathway activated by a receptor selected from the group consisting of an interleukin receptor, a cytokine receptor, an interferon receptor and a growth factor receptor. In another embodiment the signaling pathway is a JAK/STAT signaling pathway or a MAP kinase signaling pathway.

Cytokines secreted by tumor cells can have several effects on tumor growth. Several cytokines such as IL-10 or TGF-β can suppress anti-tumor immune response leading to the establishment of immunological tolerance to tumor antigens. Tumor cells may also express functional receptor complex for a cytokine secreted by the same tumor cells. If this cytokine can support proliferation of tumor cells, for example through the activation of STAT3 and/or STAT5, then this cytokine may function as an autocrine growth factor for tumor cells.

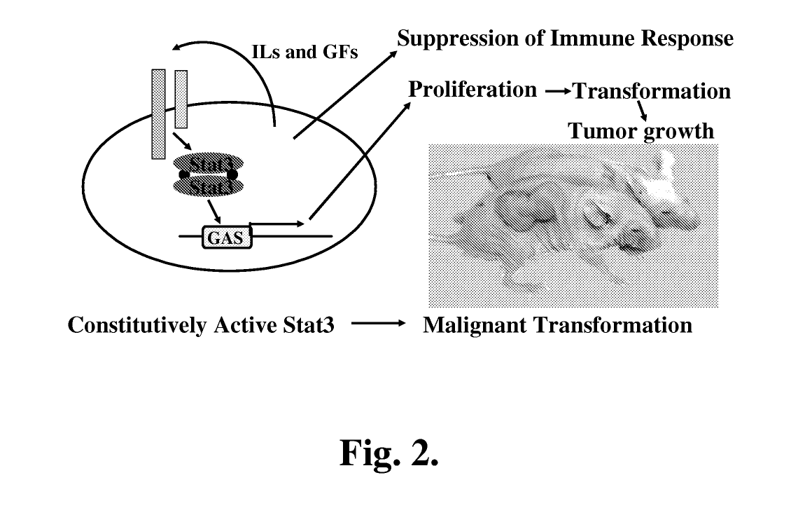

Many interleukins (ILs) and growth factors (GFs) as well as several oncoproteins are able to predominantly activate STAT3 during signal transduction events. Constitutive signaling through STAT3 leads to malignant transformation (FIG. 2). Cytokine milieu can shift the balance between STAT1 and STAT3 activation inside of tumor cells. IFNs are well known for their ability to signal predominantly through STAT1 activation that leads to the induction of anti-proliferative response, cell cycle arrest and apoptosis.

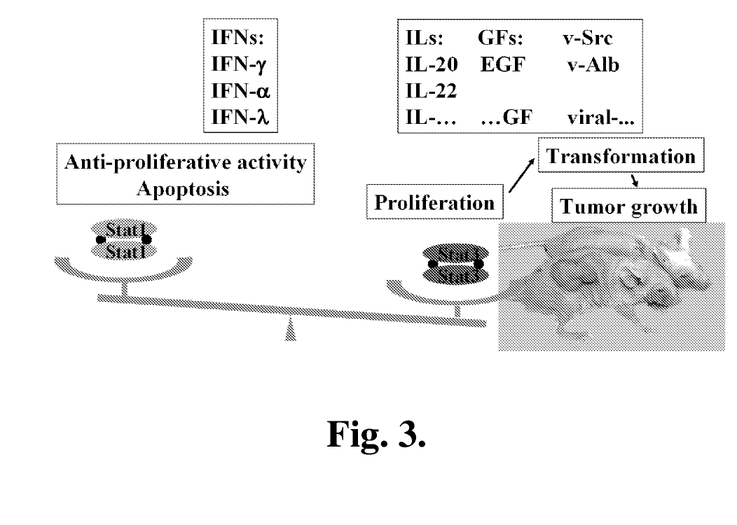

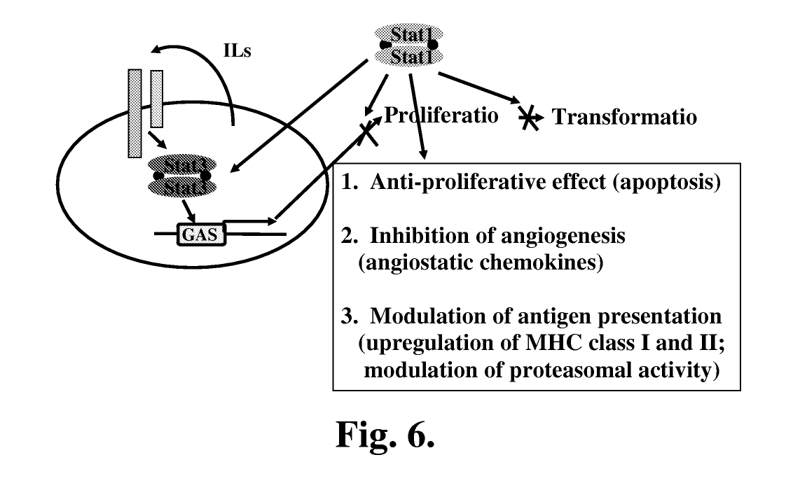

To circumvent immune surveillance, tumors must develop means of protecting themselves from being recognized by immune cells as an abnormal cell type thus avoiding destruction by the immune cells. Often this leads to general suppression of the immune system. In addition, tumor cells constitutively proliferate. Agents inducing constitutive proliferation are often driving forces in cancer development. Autocrine growth factors or mutations leading to activation of one or more signal transduction pathways (e.g., Ras-MAP kinase or Jak-STAT pathways) induced by growth factors are examples of such agents. The present invention includes a method to deliver anti-proliferative signals to cancer cells forcing them to stop or at least to slow down their proliferation. For example, expression of constitutively active STAT3 was shown to drive cells to malignant transformation (Bromberg et al., 1999). Studies also demonstrated that STAT3 is maintained in activated form in many tumors. In contrast, activation of STAT1 is linked to anti-proliferative effect, cell cycle arrest and induction of apoptosis (FIGS. 3 and 6). Therefore, a method was designed to force tumor cells to express constitutively active STAT1. STAT1 is predominantly activated by interferons (IFNs) (FIG. 3). IFNs are also well known for their strong anti-proliferative activities. Therefore, a constitutively active IFN signaling was designed. The chimeric receptors shown in FIGS. 1 C and D are examples of such IFN signaling constructs.

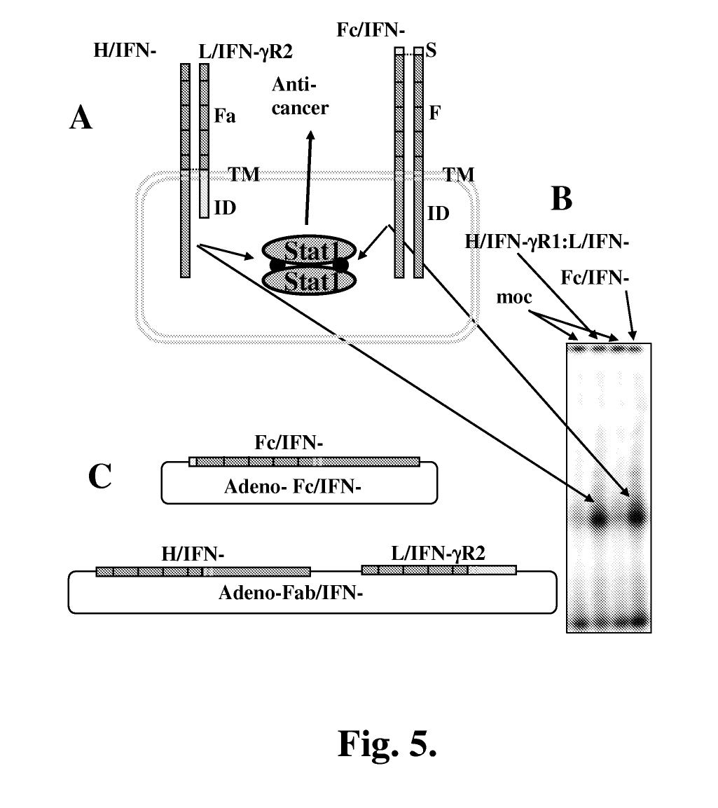

In another aspect the present invention is directed to a method of preventing, treating, or ameliorating a disease related to an increased or extended signaling pathway induced by cytokines or growth factors in a subject comprising administering to the subject a composition according of the present invention wherein the composition activates a signaling pathway that counterbalances, suppresses or alters the increased or extended signaling pathway in the subject. In one embodiment the disease is selected from the group consisting of cancer, malignant conditions, chronic infections with various pathogens such as viruses and bacteria, chronic inflammatory conditions and autoimmune diseases. In another aspect the present invention is directed to a method of preventing, treating, or ameliorating a disease related to increased or extended STAT3 activity in a subject comprising administering to the subject a composition according to the present invention wherein the composition activates a STAT1 signaling pathway in the subject. Many cancer cells demonstrate constitutively active STAT3 signaling that can be induced through several mechanisms (FIG. 3) including response to various interleukins (ILs) present in the local environment (cytokine milieu). By forcing cells to undergo constitutive IFN signaling (FIG. 5) resulting in STAT1 activation, the balance between activated STAT3 and STAT1 is shifted toward STAT1. This event leads to the induction of various biological activities (some are listed on the figure) that are beneficial for immune system and disadvantageous for cancer progression.

In another aspect the present invention is directed to a chimeric polypeptide comprising a first portion comprising a receptor domain, wherein the receptor domain comprises a receptor extracellular region; and a second portion comprising a dimerization domain, wherein the dimerization domain comprises an antibody heavy chain region of a Fab fragment or an antibody light chain region. In one embodiment, the first portion consists of the receptor domain and the second portion consists of the dimerization domain. In another embodiment, the chimeric polypeptide comprises a linker sequence between the first portion and the second portion. In another embodiment, the receptor domain comprises amino acid sequence from a receptor selected from the group consisting of an interleukin receptor, a cytokine receptor, an interferon receptor and a growth factor receptor. In another embodiment, the receptor domain comprises an amino acid sequence selected from the group consisting of SEQ ID NO: 51 to 55. In another embodiment, the dimerization domain comprises an amino acid sequence selected from the group consisting of SEQ ID NO: 26 to 30.

In another aspect the present invention is direct to a composition comprising first chimeric polypeptide wherein the first chimeric polypeptide is a chimeric polypeptide comprising a first portion comprising a receptor domain, wherein the receptor domain comprises a receptor extracellular region; and a second portion comprising a dimerization domain, wherein the dimerization domain comprises an antibody heavy chain region of a Fab fragment or an antibody light chain region; and a second chimeric polypeptide wherein the second chimeric polypeptide is a chimeric polypeptide comprising a first portion comprising a receptor domain, wherein the receptor domain comprises a receptor extracellular region; and a second portion comprising a dimerization domain, wherein the dimerization domain comprises an antibody heavy chain region of a Fab fragment or an antibody light chain region, wherein the receptor domain of the second chimeric polypeptide is derived from a receptor different from the receptor domain of the first chimeric polypeptide; wherein the dimerization domain of the first chimeric polypeptide binds to the dimerization domain of the second chimeric polypeptide resulting in the receptor domain of the first chimeric polypeptide associating with the receptor domain of the second chimeric polypeptide.

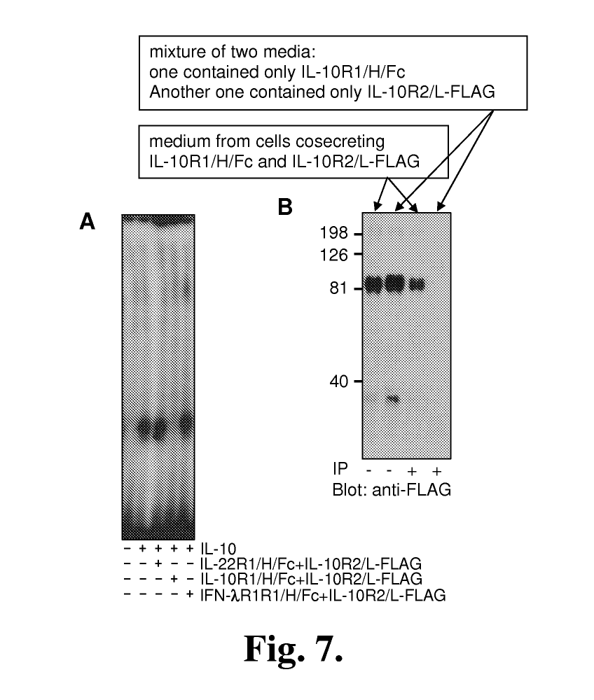

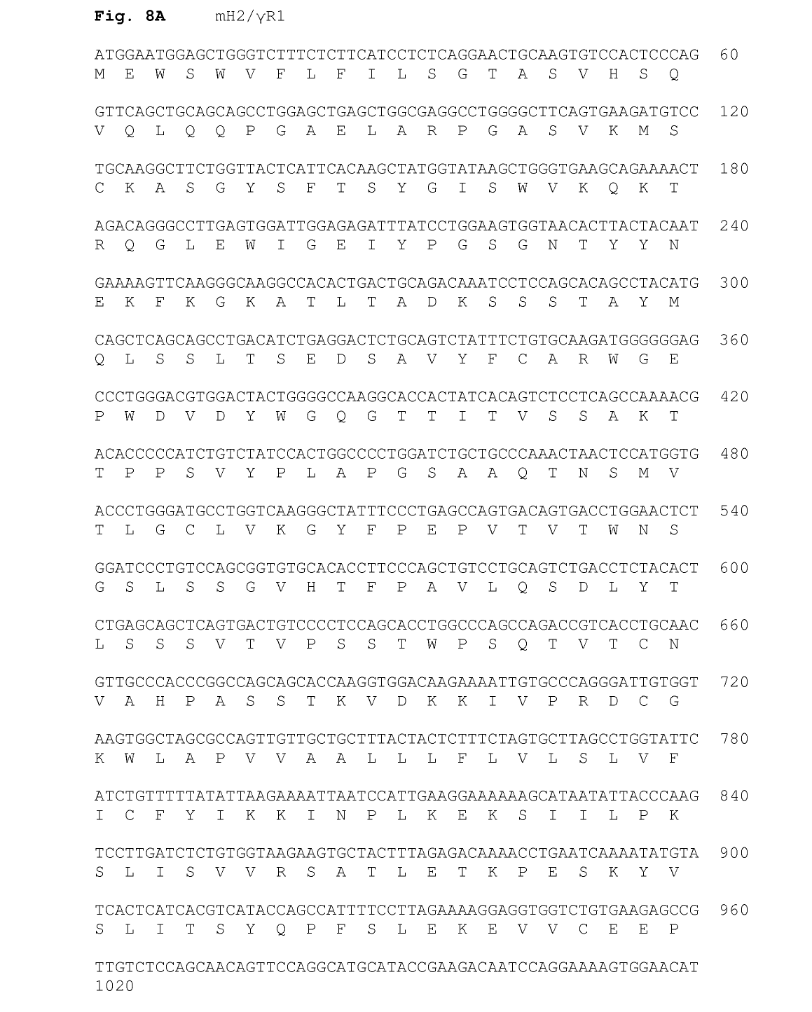

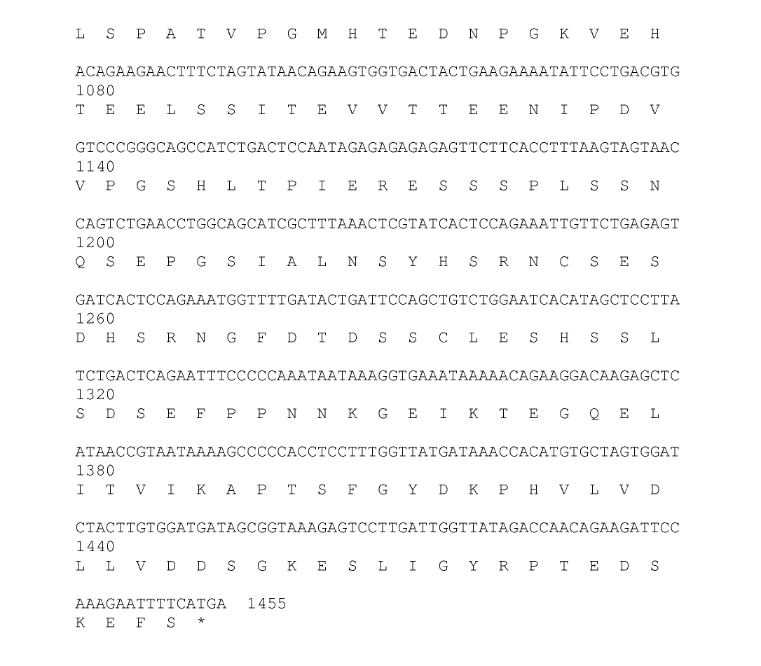

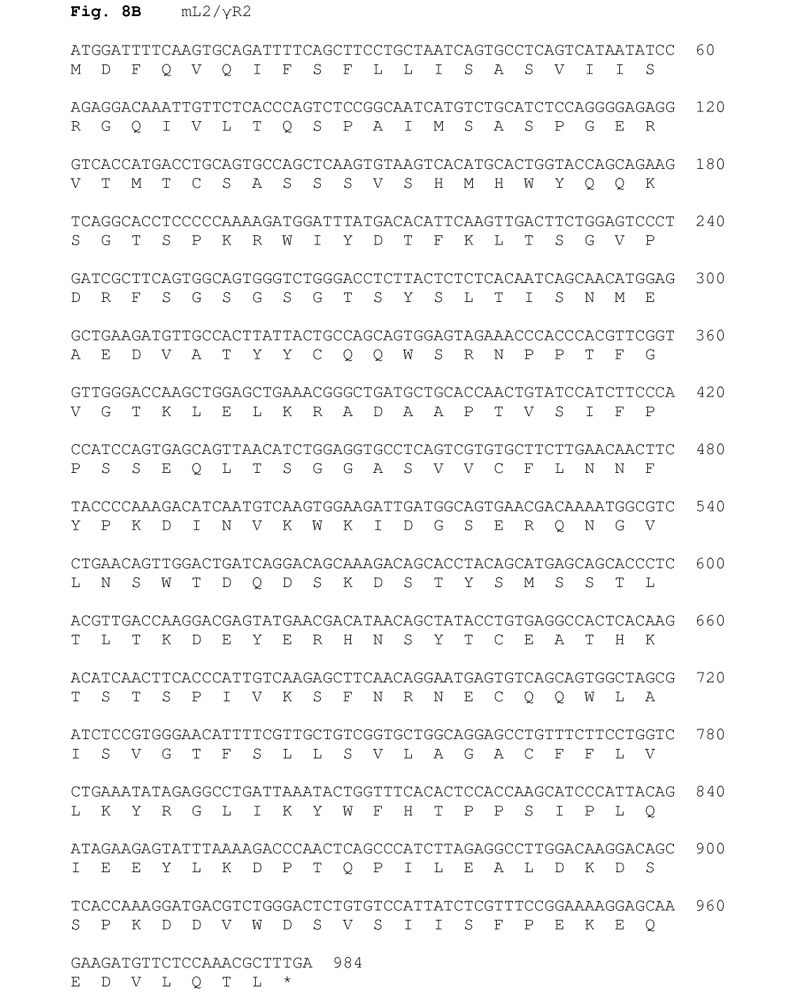

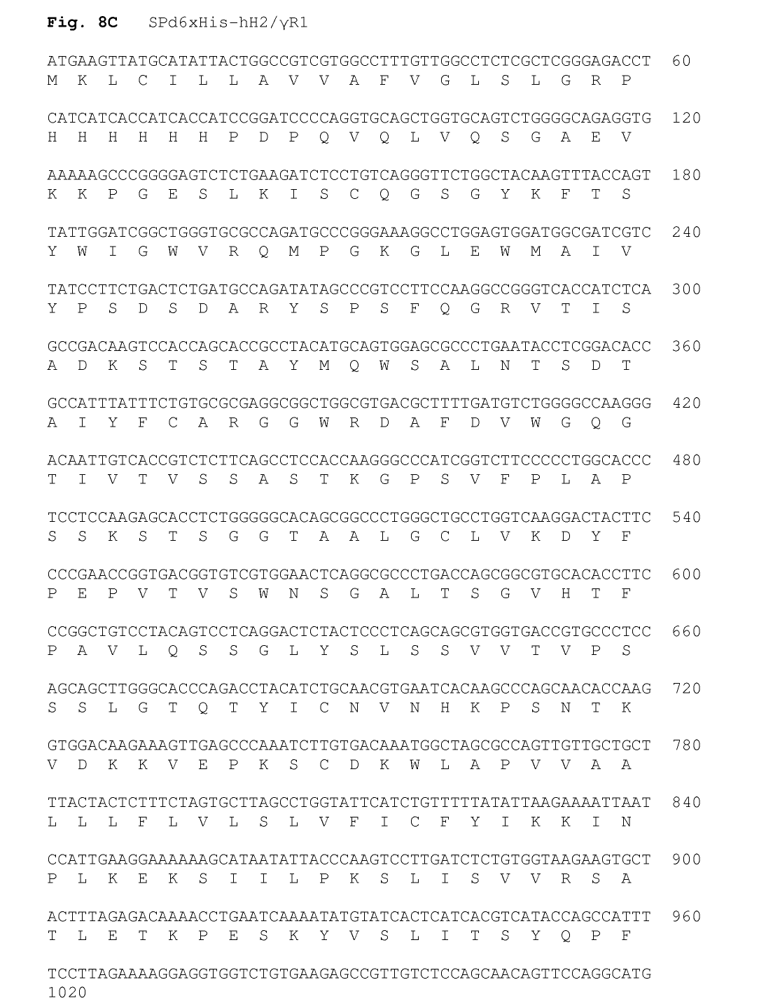

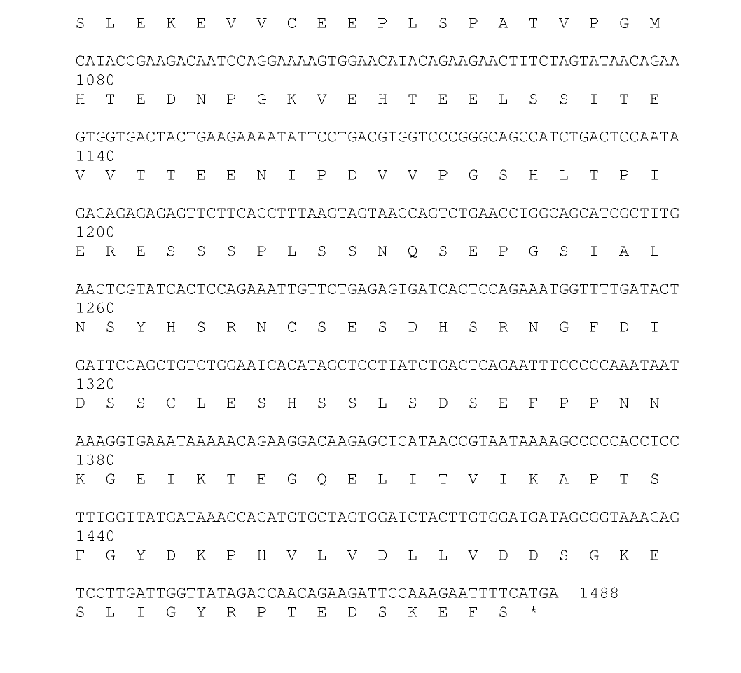

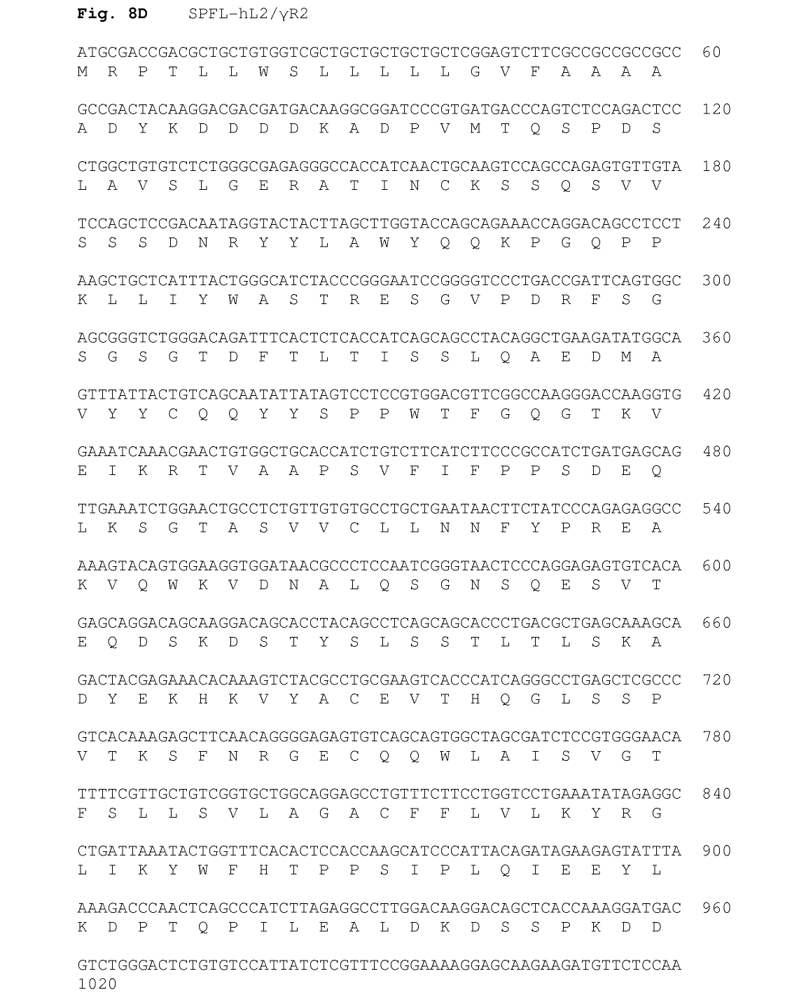

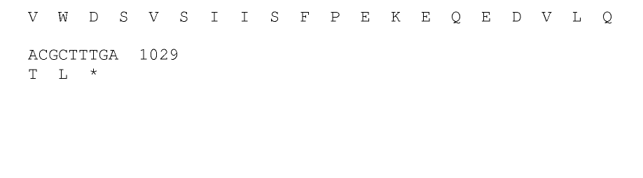

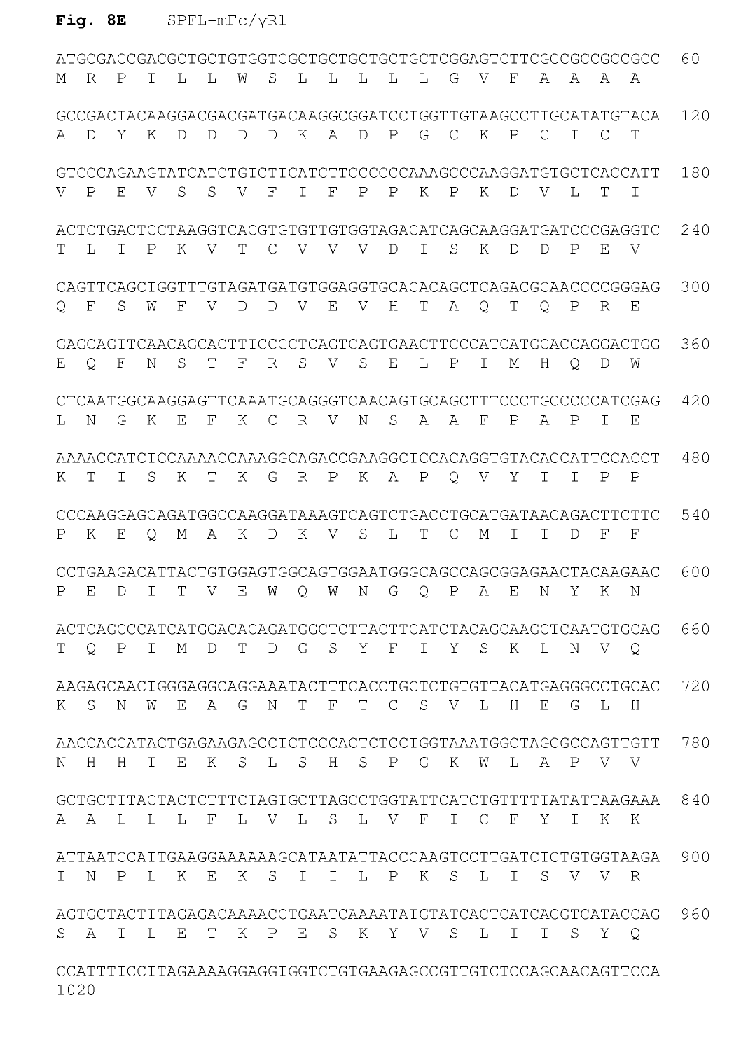

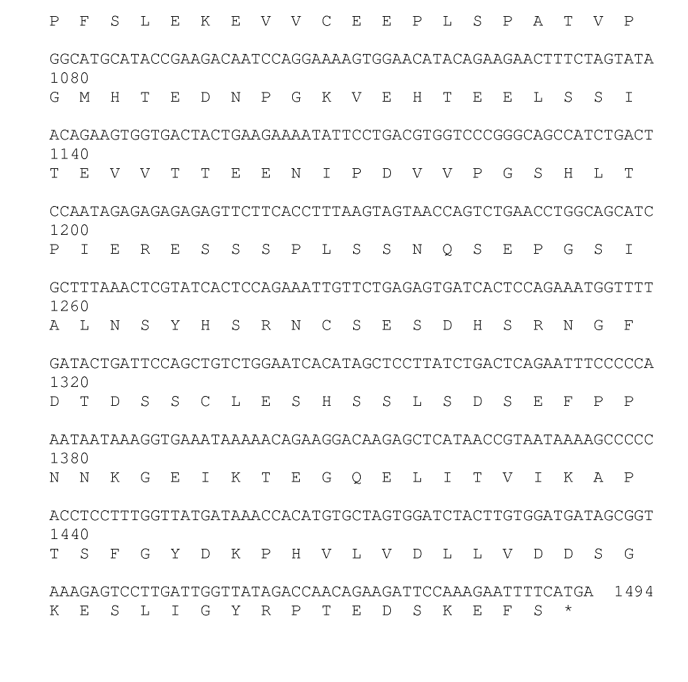

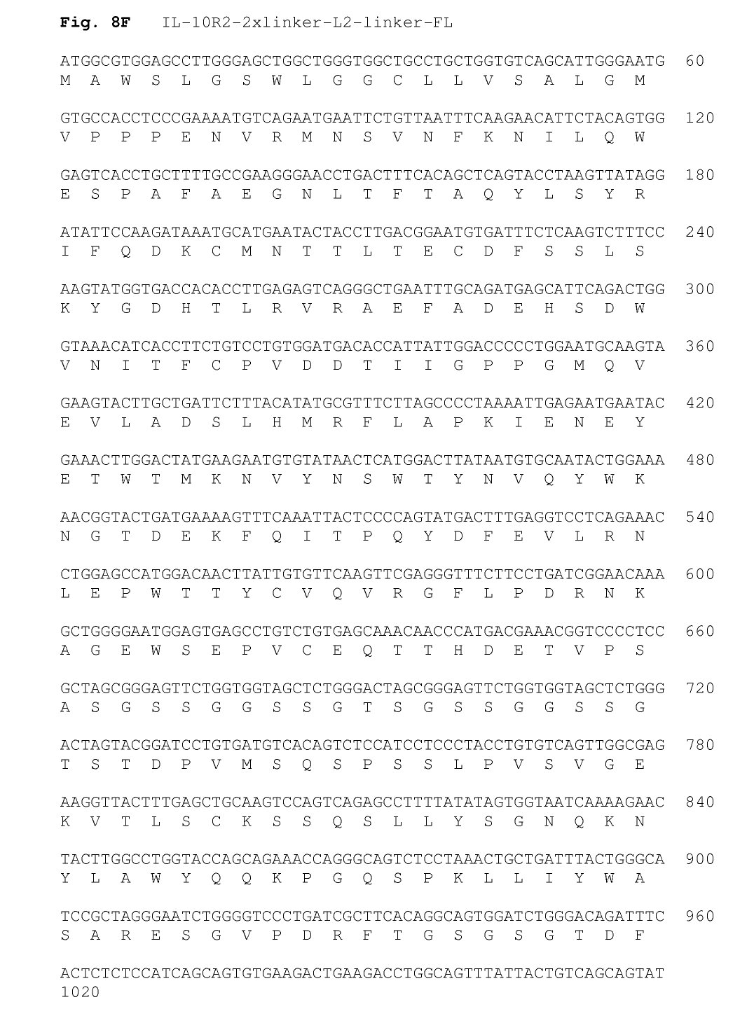

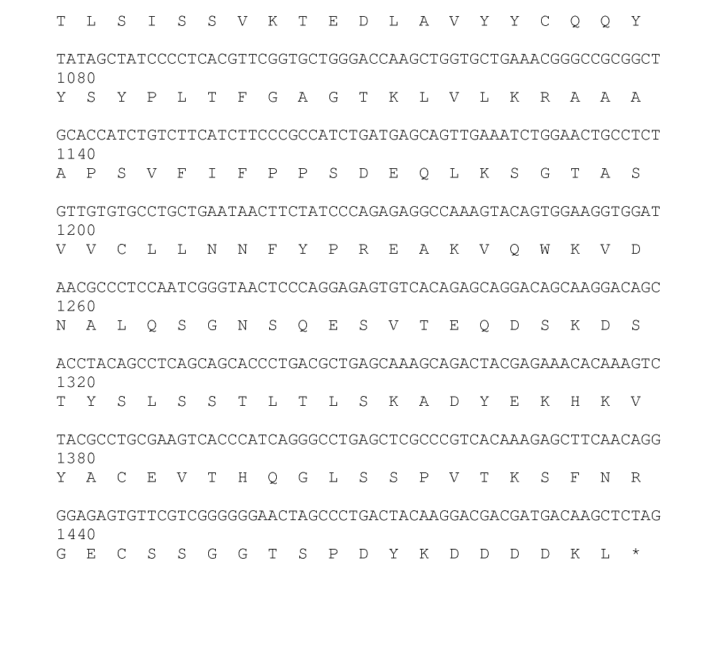

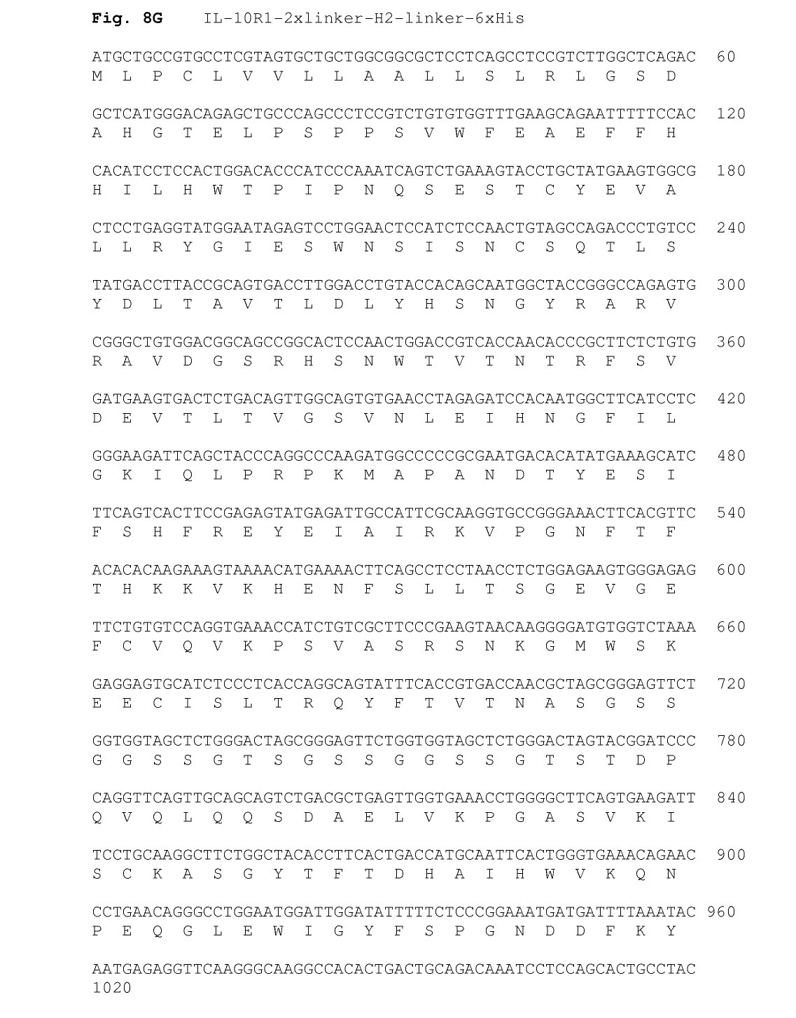

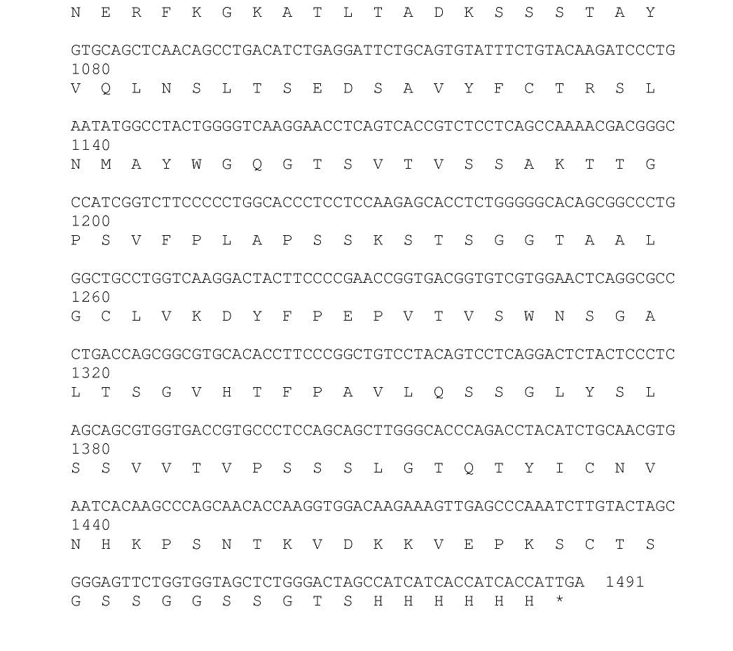

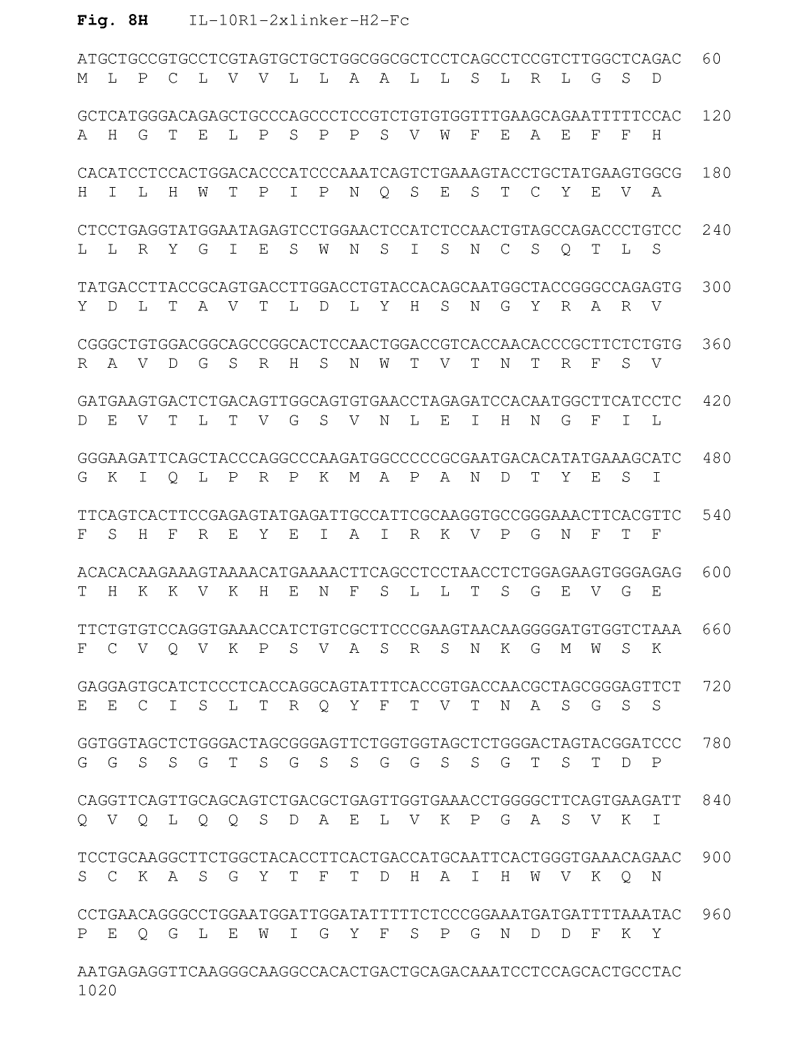

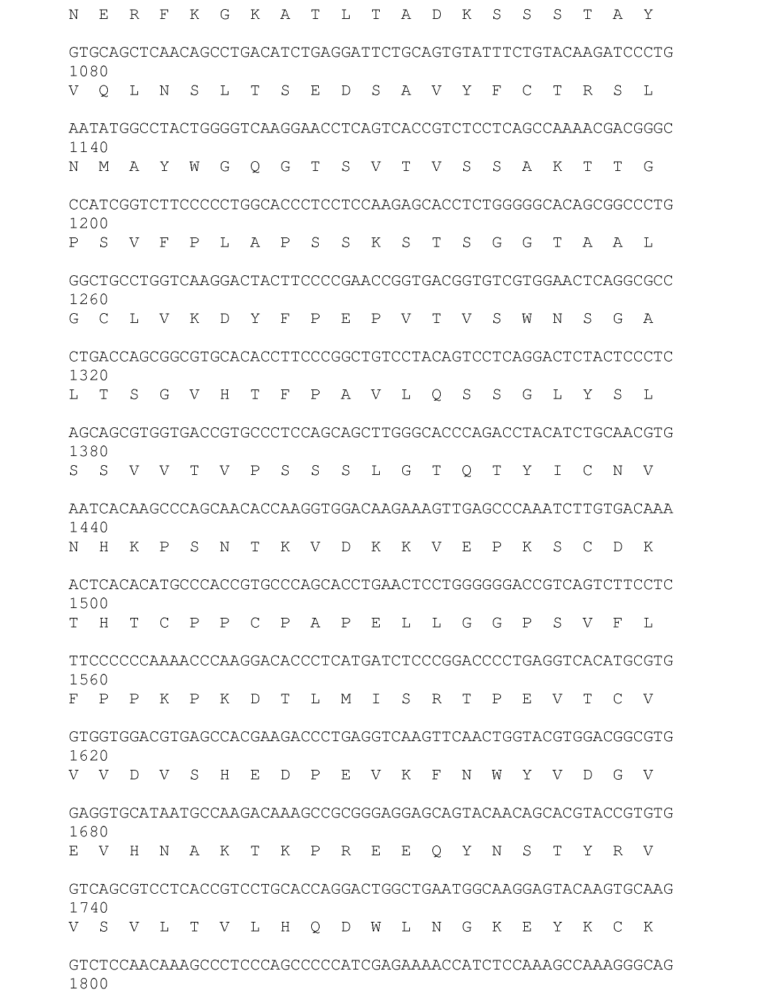

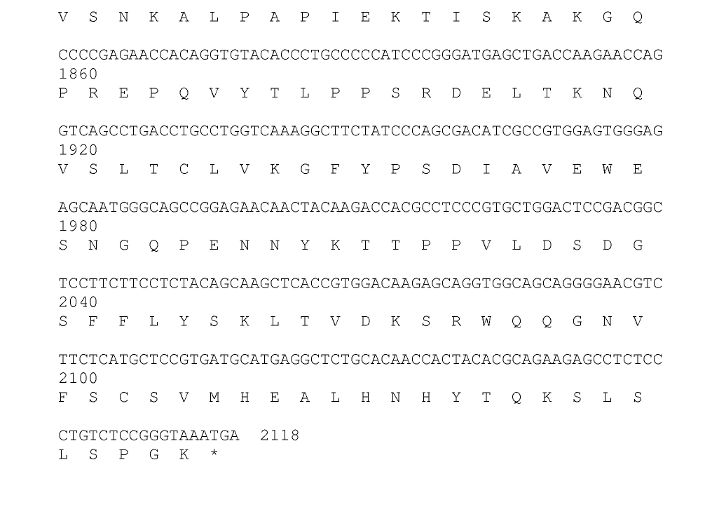

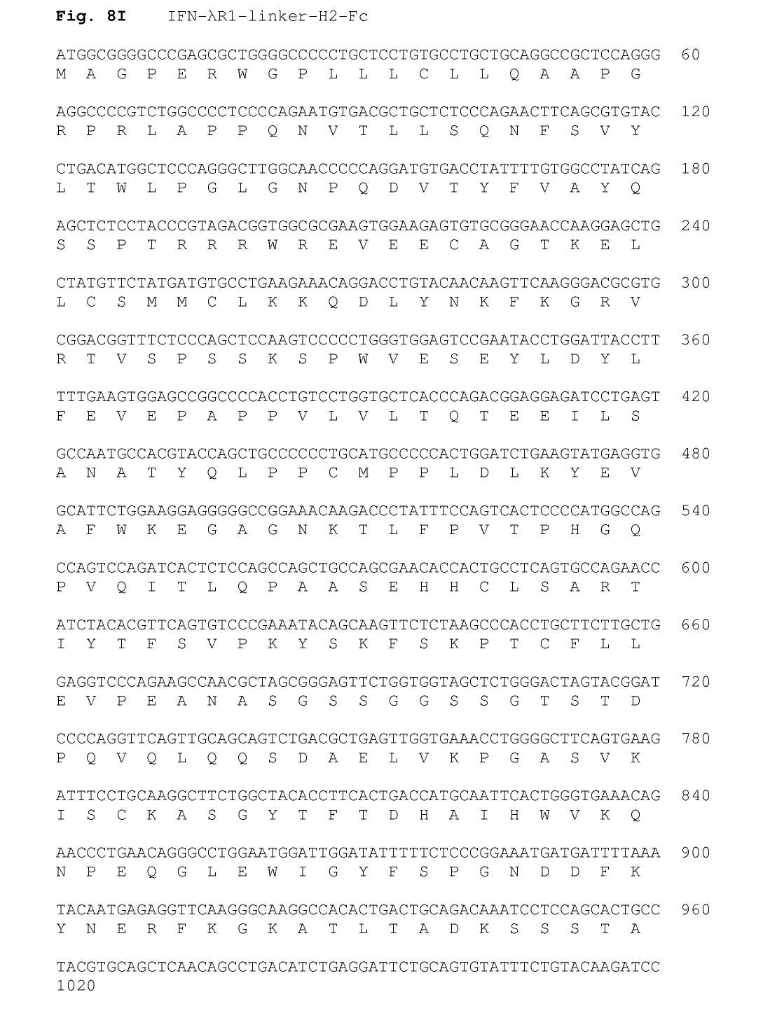

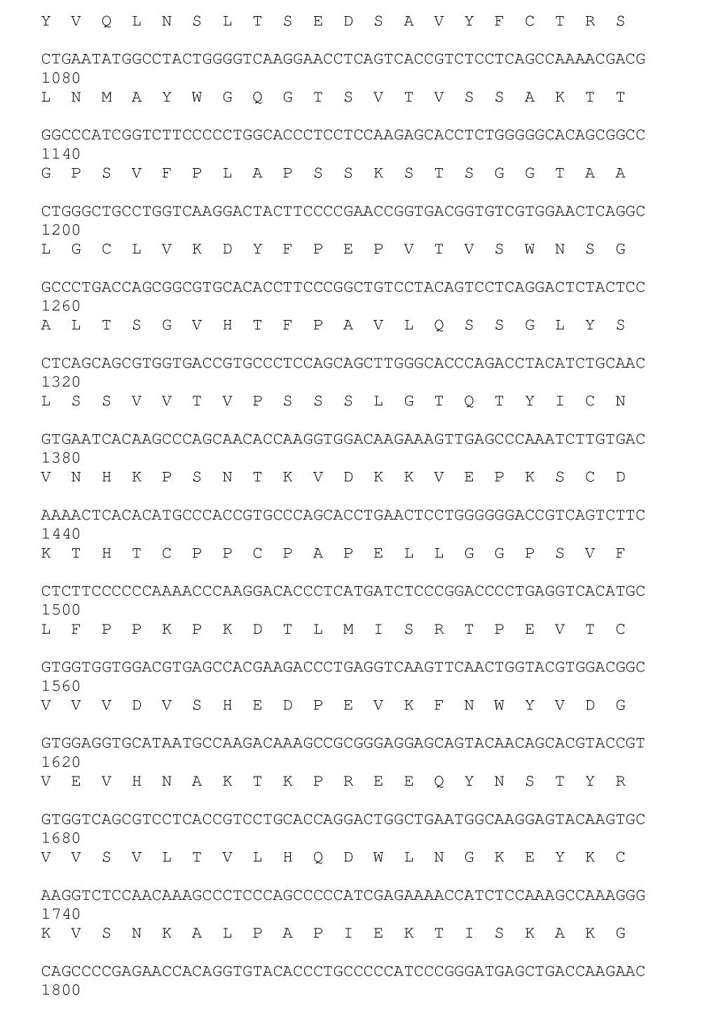

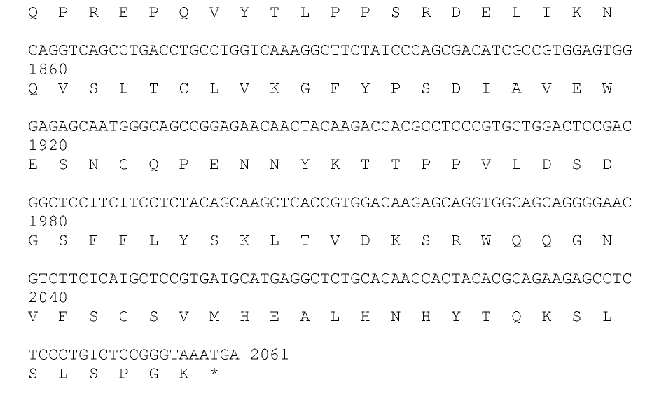

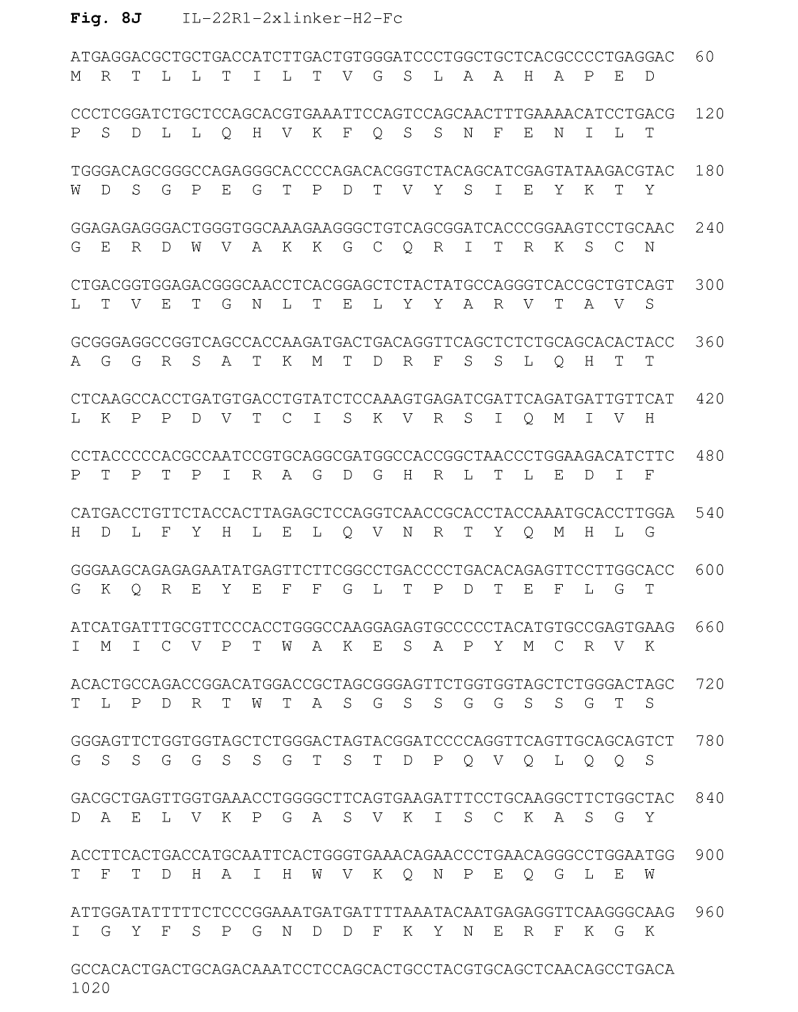

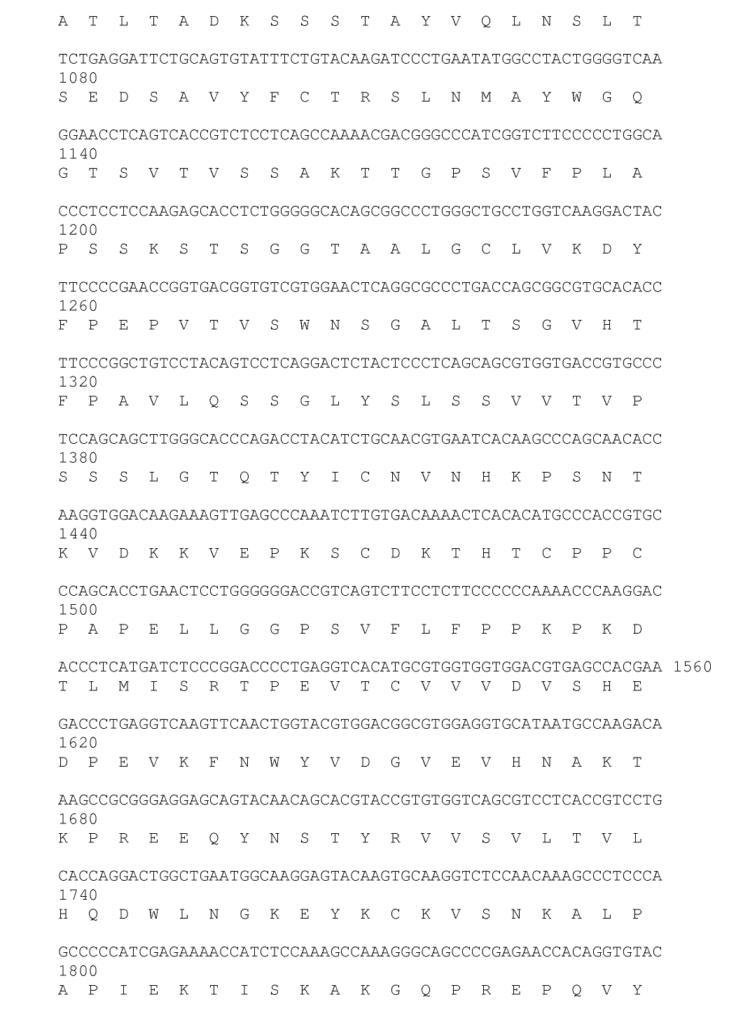

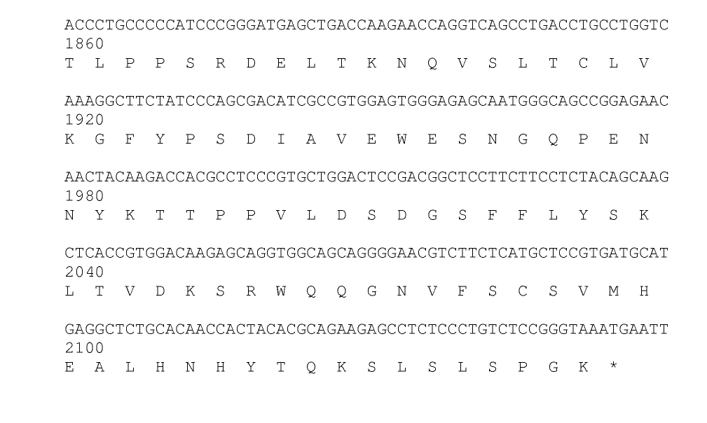

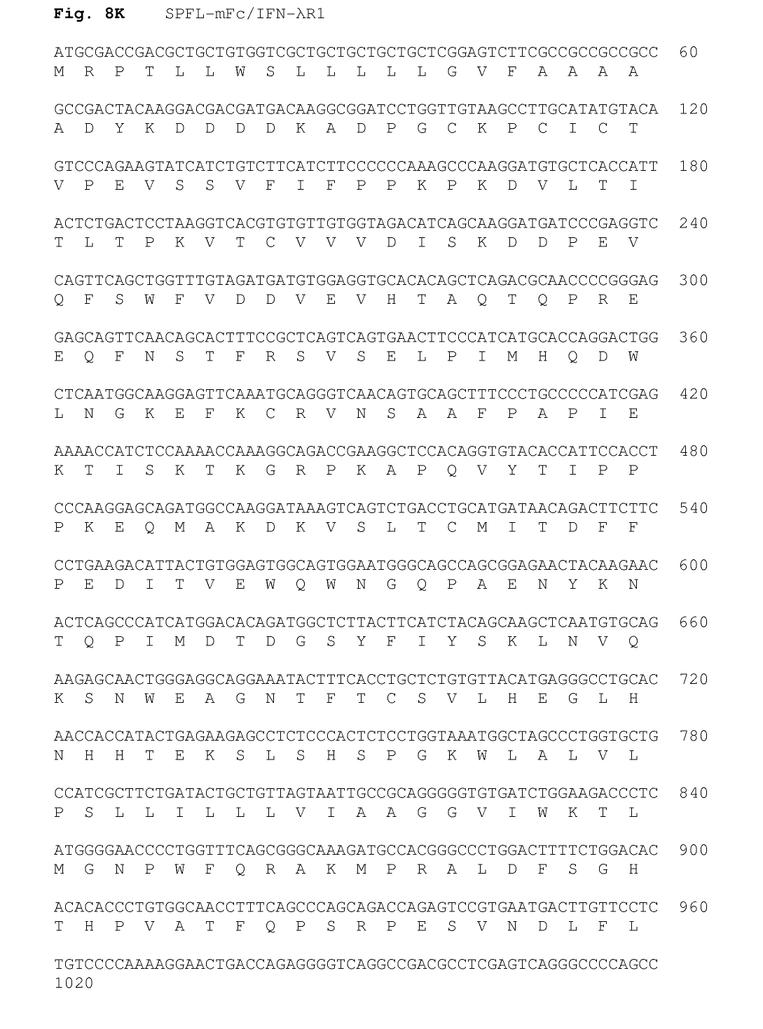

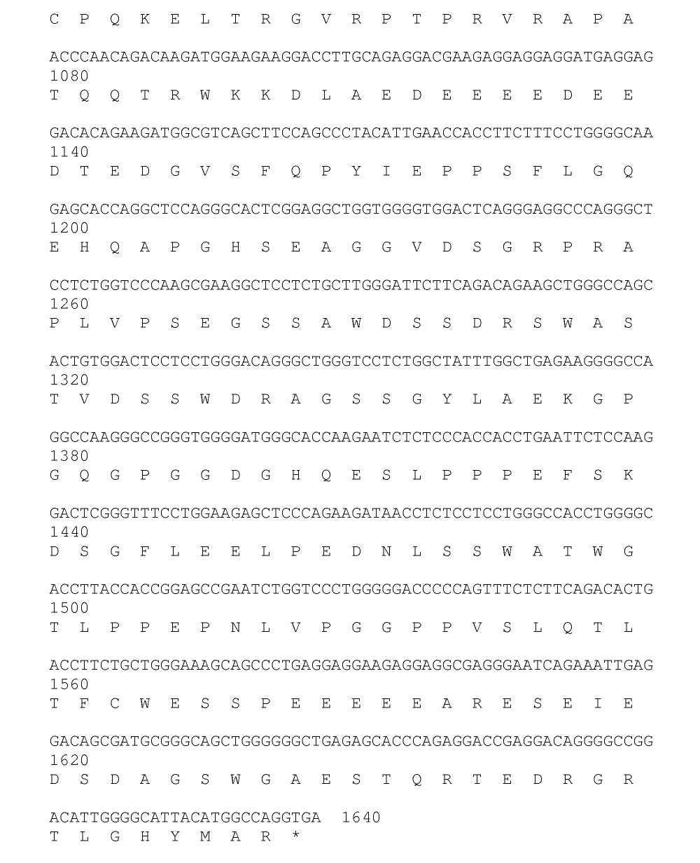

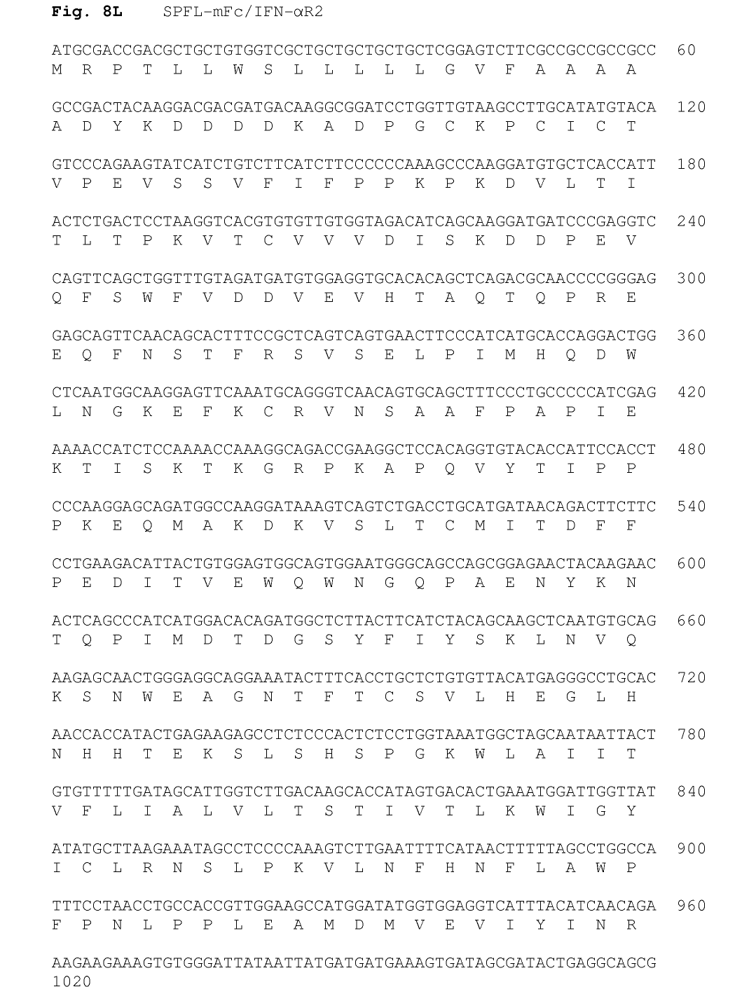

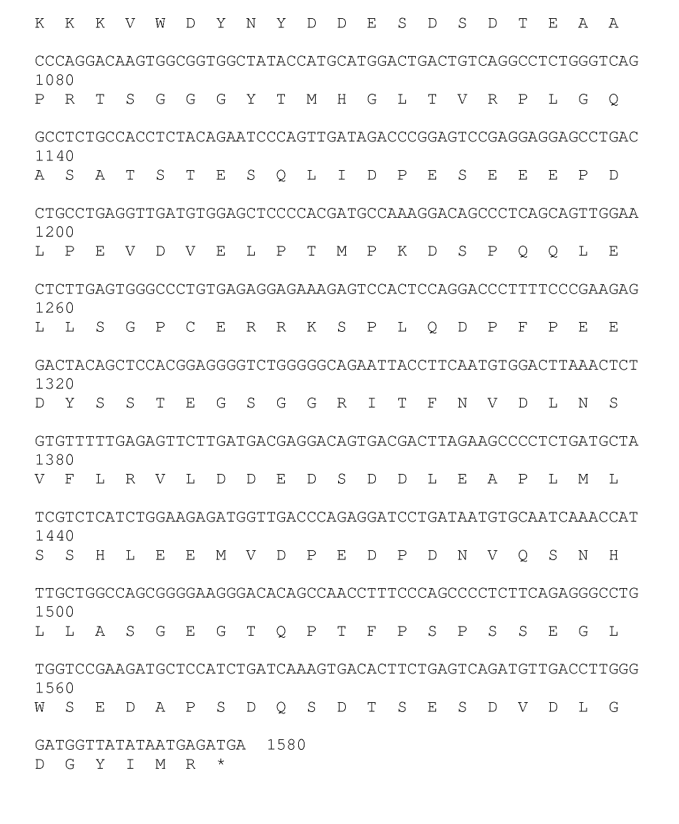

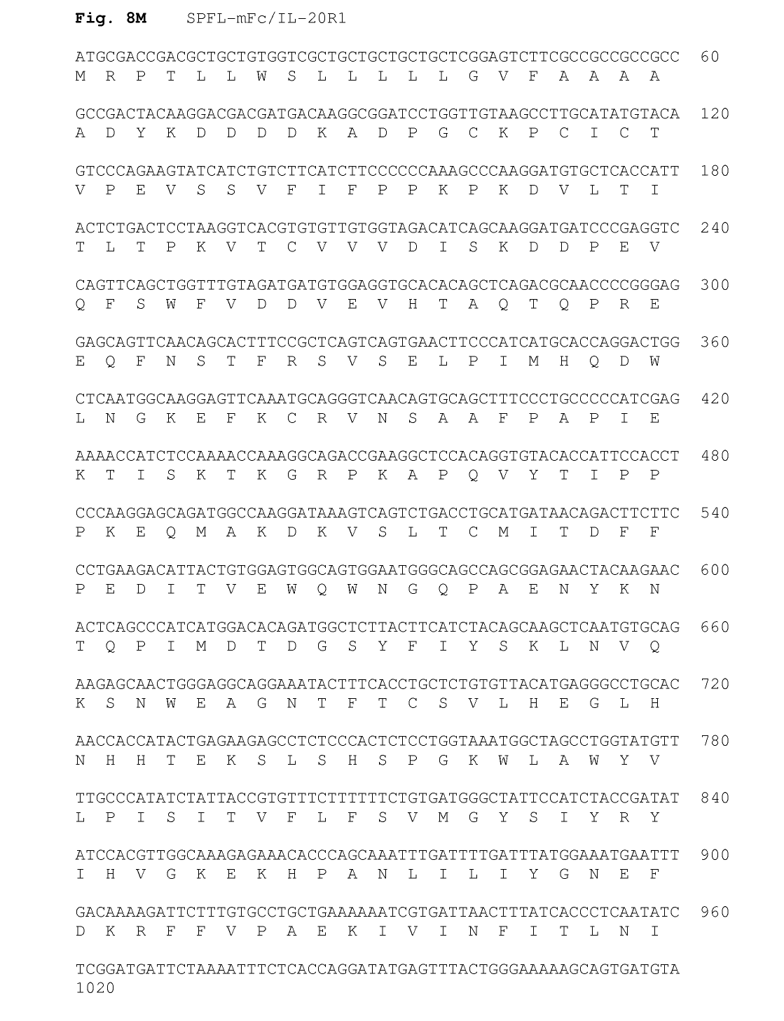

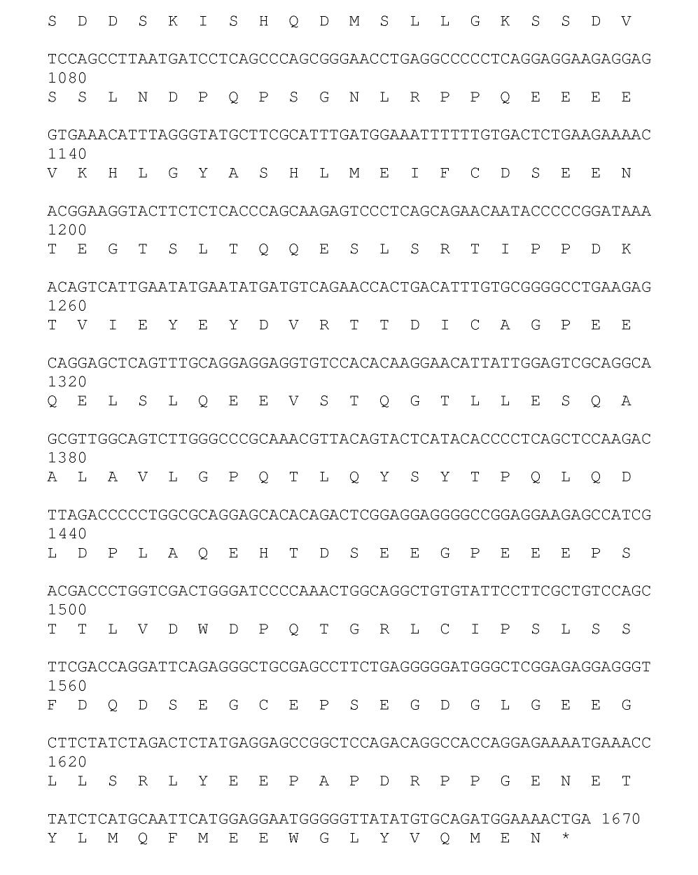

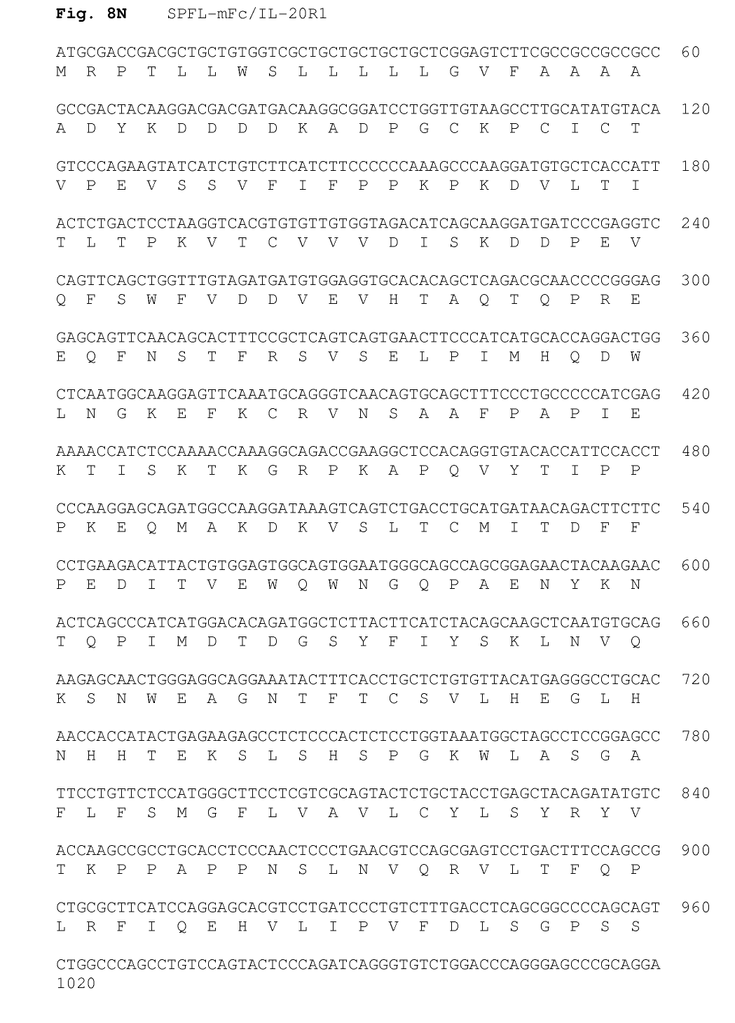

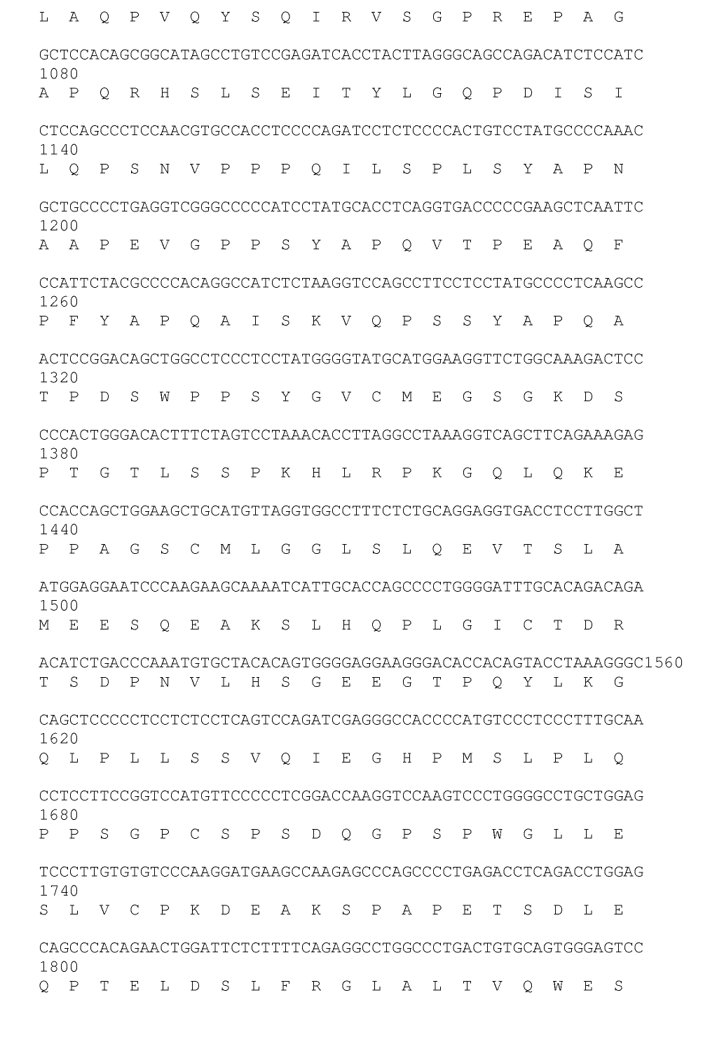

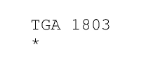

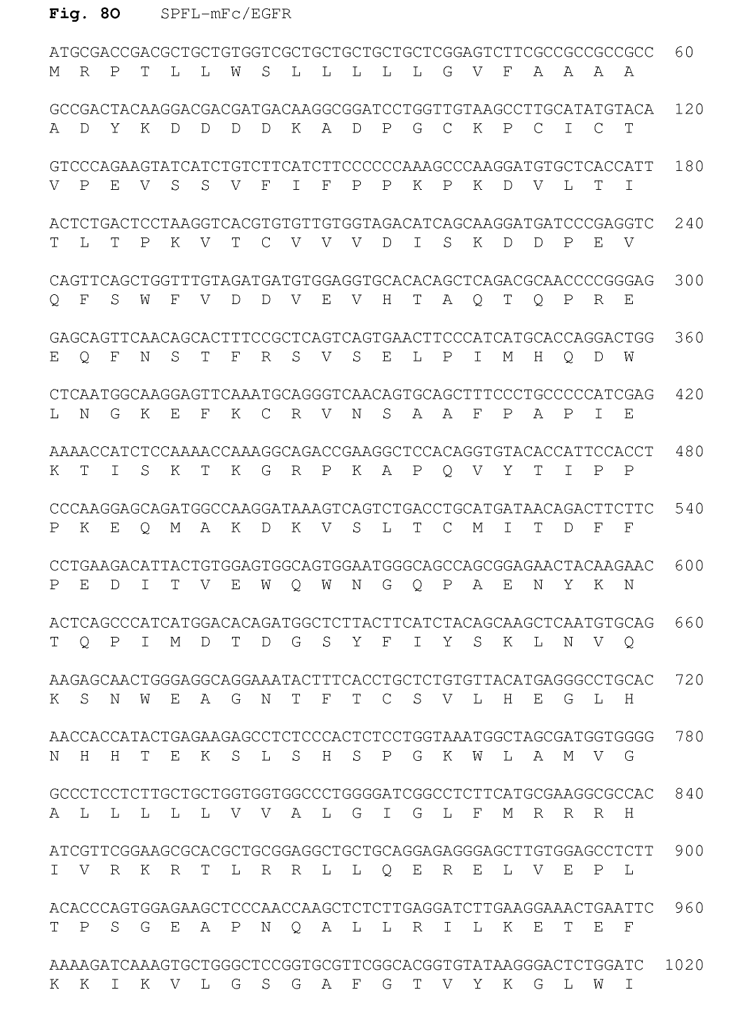

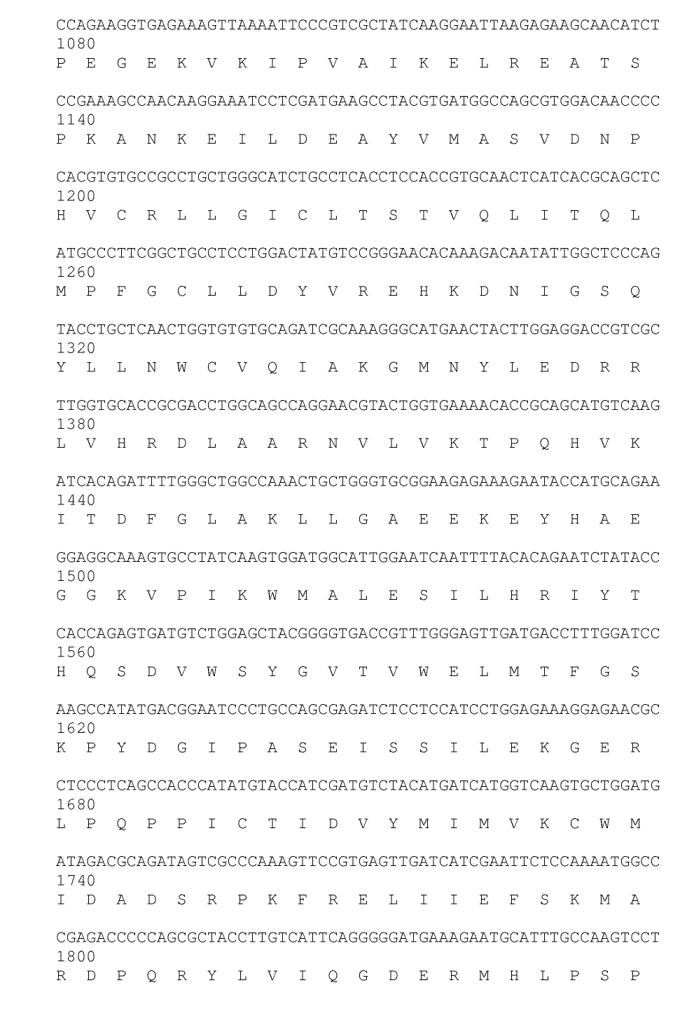

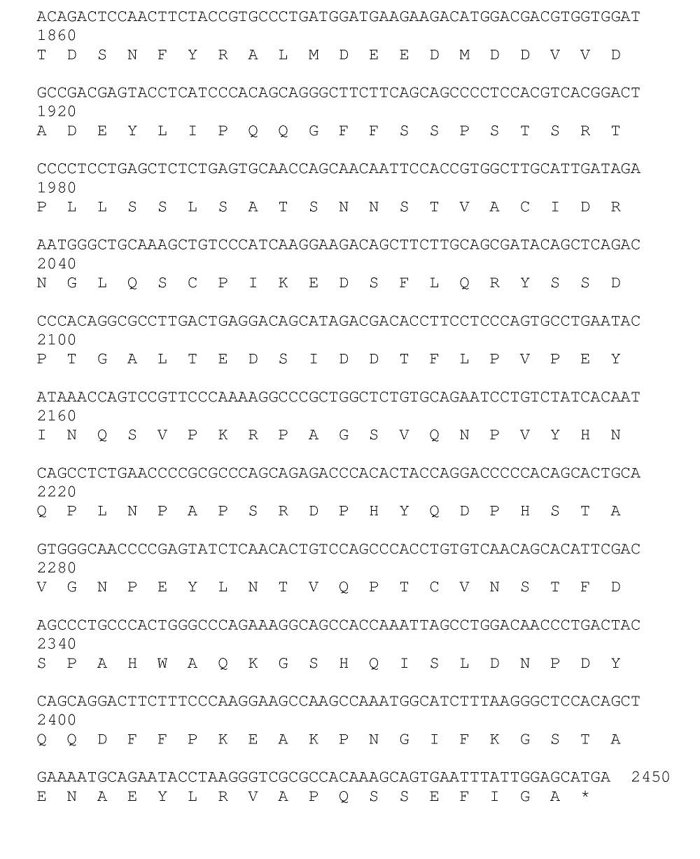

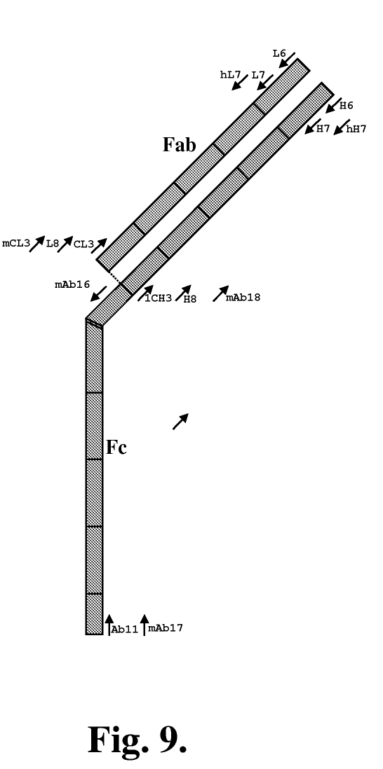

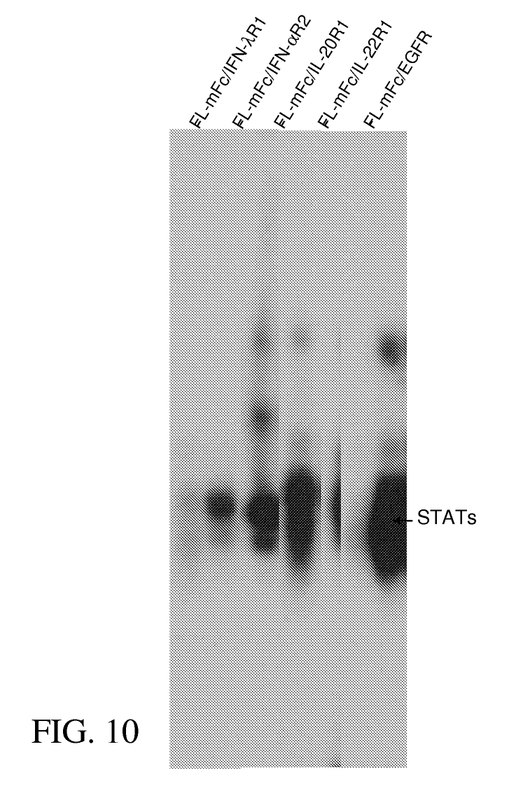

Most cytokines signal through heterodimeric receptor complexes. In general, a homodimeric receptor has lower affinity for the cytokine then the combination of two different receptor subunits. Therefore, soluble receptors representing just one receptor subunit are very inefficient in neutralizing cytokine functions. The use of Ab-receptor chimeric polypeptides as presented on FIGS. 1 E and F allows the generation of heterodimeric soluble receptors. This technique has been successfully applied to the IL-10 ligand-receptor system. IL-10R-Ab fusion molecule (IL-10R1/H/Fc+IL-10R2/L) is very efficient in specifically inhibiting IL-10 signaling (FIG. 7A). Both IL-10R1/H/Fc and IL-10R2/L chimeric chains are coexpressed in a cell to allow proper assembling of the IL-10R-Ab complex.

In another aspect the present invention is direct to a method of inhibiting or reducing activation of a signaling pathway in a cell comprising administering to the cell a composition comprising first chimeric polypeptide wherein the first chimeric polypeptide is a chimeric polypeptide comprising a first portion comprising a receptor domain, wherein the receptor domain comprises a receptor extracellular region; and a second portion comprising a dimerization domain, wherein the dimerization domain comprises an antibody heavy chain region of a Fab fragment or an antibody light chain region; and a second chimeric polypeptide wherein the second chimeric polypeptide is a chimeric polypeptide comprising a first portion comprising a receptor domain, wherein the receptor domain comprises a receptor extracellular region; and a second portion comprising a dimerization domain, wherein the dimerization domain comprises an antibody heavy chain region of a Fab fragment or an antibody light chain region, wherein the receptor domain of the second chimeric polypeptide is derived from a receptor different from the receptor domain of the first chimeric polypeptide; wherein the dimerization domain of the first chimeric polypeptide binds to the dimerization domain of the second chimeric polypeptide resulting in the receptor domain of the first chimeric polypeptide associating with the receptor domain of the second chimeric polypeptide. In one embodiment the signaling pathway is activated by a cytokine. In another embodiment the cytokine is selected from the group consisting of type I IFN, type II IFN, type III IFN, IL-10, IL-19, IL-20, IL-22, IL-24, IL-26 and Epidermal Growth Factor (EGF)

Chimeric polypeptides and compositions of the present invention can be used for the treatment of autoimmune diseases, cancers, immunomodulation, and any antibody-mediated pathologies. Administration of chimeric polypeptides and compositions of the present invention to a subject can be intravenous, intraarterial, intraperitoneal, intramuscular, subcutaneous, intrapleural, intrathecal, by perfusion through a regional catheter, or by direct intralesional injection. When administering therapeutic proteins by injection, the administration may be by continuous infusion or by single or multiple boluses.

Additional routes of administration include oral, mucosal-membrane, pulmonary, and transcutaneous. A pharmaceutical composition comprising a chimeric polypeptides and compositions of the present invention can be formulated according to known methods to prepare pharmaceutically useful compositions, whereby the chimeric polypeptides and compositions of the present invention are combined in a mixture with a pharmaceutically acceptable carrier. A composition is to be a “pharmaceutically acceptable carrier” if its administration can be tolerated by a recipient patient. Sterile phosphate-buffered saline is one example of a pharmaceutically acceptable carrier. Other suitable carriers are well-known to those in the art.

For purposes of therapy, chimeric polypeptides and compositions of the present invention and a pharmaceutically acceptable carrier are administered to a patient in a therapeutically effective amount. A combination of a chimeric polypeptides and compositions of the present invention and a pharmaceutically acceptable carrier is to be administered in a “therapeutically effective amount” if the amount administered is physiologically significant. An agent is physiologically significant if its presence results in a detectable change in the physiology of a recipient patient. For example, an agent used to treat inflammation is physiologically significant if its presence alleviates the inflammatory response. As another example, an agent used to inhibit the growth of tumor cells is physiologically significant if the administration of the agent results in a decrease in the number of tumor cells, decreased metastasis, a decrease in the size of a solid tumor, or increased necrosis of a tumor.

A pharmaceutical composition comprising chimeric polypeptides and compositions of the present invention can be furnished in liquid form, in an aerosol, or in solid form. Liquid forms, are illustrated by injectable solutions and oral suspensions. Exemplary solid forms include capsules, tablets, and controlled-release forms.

Liposomes provide one means to deliver therapeutic polypeptides to a subject intravenously, intraperitoneally, intrathecally, intramuscularly, subcutaneously, or via oral administration, inhalation, or intranasal administration. Liposomes are microscopic vesicles that consist of one or more lipid bilayers surrounding aqueous compartments. Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 μm to greater than 10 μm. A variety of agents can be encapsulated in liposomes: hydrophobic agents partition in the bilayers and hydrophilic agents partition within the inner aqueous space(s). Moreover, it is possible to control the therapeutic availability of the encapsulated agent by varying liposome size, the number of bilayers, lipid composition, as well as the charge and surface characteristics of the liposomes.

Liposomes can also be prepared to target particular cells or organs by varying phospholipid composition or by inserting receptors or ligands into the liposomes. For example, liposomes, prepared with a high content of a nonionic surfactant, have been used to target the liver (Hayakawa et al., Japanese Patent 04-244,018; Kato et al., Biol. Pharm. Bull. 16:960 (1993)). These formulations were prepared by mixing soybean phosphatidylcholine, a-tocopherol, and ethoxylated hydrogenated castor oil (HCO-60) in methanol, concentrating the mixture under vacuum, and then reconstituting the mixture with water. A liposomal formulation of dipalmitoylphosphatidylcholine (DPPC) with a soybean-derived sterylglucoside mixture (SG) and cholesterol (Ch) has also been shown to target the liver (Shimizu et al., Biol. Pharm. Bull. 20:881 (1997)).

Alternatively, various targeting ligands can be bound to the surface of the liposome, such as antibodies, antibody fragments, carbohydrates, vitamins, and transport proteins. In one embodiment of the present invention a first chimeric polypeptide comprising a heavy chain of a Fab fragment associates with a second chimeric polypeptide comprising a light of a Fab fragment such that upon dimerization the heavy and light chains can bind to a target molecule. In one embodiment the target molecule is found on the surface of a target cell.

In another aspect the present invention is directed to a polynucleotide encoding a chimeric polypeptide according to the present invention. In one embodiment the polynucleotide encodes a receptor domain comprising an amino acid sequence selected from the group consisting of SEQ ID NO: 41 to 50 or a receptor domain comprising an amino acid sequence selected from the group consisting of SEQ ID NO: 51 to 55. In another embodiment the polynucleotide encodes a dimerization domain comprising an amino acid sequence selected from the group consisting of SEQ ID NO: 16 to 25 or a dimerization domain comprising an amino acid sequence selected from the group consisting of SEQ ID NO: 26 to 30. In another embodiment the polynucleotide the polynucleotide comprises a sequence selected from the group consisting of SEQ ID NO: 86 to 95 and 111 to 120 or a sequence selected from the group consisting of SEQ ID NO: 96 to 100 and 121 to 125.

In another aspect the present invention is directed to a vector comprising a polynucleotide sequence of the present invention. In one embodiment the vector comprises a polynucleotide wherein the polynucleotide sequence is operably linked to a promoter selected from the group consisting of HSV, TK, RSV, SV40, CMV, elongation factor 1α (EF-1α), and β-actin promoters. Expression vectors can also contain other nucleotide sequences, such as IRES elements, polyadenylation signals, splice donor/splice acceptor signals, and the like.

In another aspect the present invention is directed to a cell comprising a chimeric polypeptide, a polynucleotide or a vector of the present invention.

In another aspect the present invention is directed to a composition comprising a chimeric polypeptide, a polynucleotide or a vector of the present invention.

The present invention includes the use of polynucleotides of the present invention to provide the chimeric polypeptides of the present invention to a subject in need of treatment with the chimeric polypeptide. The present nucleic acids can be incorporated into an expression vector and subsequently used to transform, transfect or infect a suitable host cell. In such an expression vector the nucleic acid according to the invention is operably linked to a control sequence, such as a suitable promoter or the like, ensuring expression of the proteins according to the invention in a suitable host cell. The expression vector can be a plasmid, cosmid, virus or other suitable vector. The expression vector and the host cell transfected, transformed or infected with the vector also form part of the present invention. Preferably, the host cell is a eukaryotic cell or a bacterial cell and even more preferably a mammalian cell or insect cell. Mammalian host cells are particularly advantageous because they provide the necessary post-translational modifications to the expressed proteins according to the invention, such as glycosylation or the like, which modifications confer optimal biological activity of the proteins, which when isolated can advantageously be used in diagnostic kits or the like.

The recombinant vectors of the invention generally comprise a polynucleotide of the present invention operatively positioned downstream from a promoter. The promoter is capable of directing expression of the polynucleotide of the present invention in a mammalian, e.g. human cell. Such promoters are thus “operative” in mammalian cells, e.g. human cells.

Expression vectors and plasmids embodying the present invention comprise one or more constitutive promoters, such as viral promoters or promoters from mammalian genes that are generally active in promoting transcription. Examples of constitutive promoters include the HSV, TK, RSV, SV40, CMV, elongation factor 1α (EF-1α), and β-actin promoters.

Inducible promoters and/or regulatory elements are also contemplated for use with the expression vectors of the invention. Examples of suitable inducible promoters include promoters from genes such as cytochrome P450 genes, heat shock protein genes, metallothionein genes, hormone-inducible genes, such as the estrogen gene promoter, and such like. Promoters that are activated in response to exposure to ionizing radiation, such as fos, jun and erg-1, are also contemplated. The tetVP16 promoter that is responsive to tetracycline is a currently preferred example.

Tissue-specific promoters and/or regulatory elements will be useful in certain embodiments. Examples of such promoters that can be used with the expression vectors of the invention include promoters from the liver fatty acid binding (FAB) protein gene, specific for colon epithelial cells; the insulin gene, specific for pancreatic cells; the transphyretin, α-1-antitrypsin, plasminogen activator inhibitor type 1 (PAI-1), apolipoprotein AI and LDL receptor genes, specific for liver cells; the myelin basic protein (MBP) gene, specific for oligodendrocytes; the glial fibrillary acidic protein (GFAP) gene, specific for glial cells; OPSIN, specific for targeting to the eye; and the neural-specific enolase (NSE) promoter that is specific for nerve cells.

The construction and use of expression vectors and plasmids is well known to those of skill in the art. Virtually any mammalian cell expression vector can thus be used in connection with the genes disclosed herein.

Preferred vectors and plasmids are constructed with at least one multiple cloning site. In certain embodiments, the expression vector will comprise a multiple cloning site that is operatively positioned between a promoter and a human Mus81 or murine Mus81 encoding gene sequence. Such vectors can be used, in addition to uses in other embodiments, to create N-terminal or C-terminal fusion proteins by cloning a second protein-encoding DNA segment into the multiple cloning site so that it is contiguous and in-frame with the mammalian Mus81 encoding nucleotide sequence.

There are numerous approaches to introduce a gene to a subject, including the use of recombinant host cells that express, delivery of naked nucleic acid, use of a cationic lipid carrier with a nucleic acid molecule, and the use of viruses, such as recombinant retroviruses, recombinant adeno-associated viruses, recombinant adenoviruses, and recombinant Herpes simplex viruses. In an ex vivo approach, for example, cells are isolated from a subject, transfected with a vector that expresses the chimeric polypeptides of the present invention, and then transplanted into the subject.

In order to effect expression of a chimeric polypeptide of the present invention, an expression vector is constructed in which a nucleotide sequence encoding a chimeric polypeptide of the present invention is operably linked to a core promoter, and optionally a regulatory element, to control gene transcription.

Alternatively, a polynucleotide encoding a chimeric polypeptide of the present invention can be delivered using recombinant viral vectors, including for example, adenoviral vectors, adenovirus-associated viral vectors, alphaviruses such as Semliki Forest Virus and Sindbis Virus, herpes viral vectors, parvovirus vectors, pox virus vectors, pox viruses, such as canary pox virus or vaccinia virus, and retroviruses. Within various embodiments, either the viral vector itself, or a viral particle which contains the viral vector may be utilized.

Alternatively, an expression vector encoding a chimeric polypeptide of the present invention can be introduced into a subject's cells by lipofection in vivo using liposomes. Synthetic cationic lipids can be used to prepare liposomes for in vivo transfection of a gene encoding a marker. The use of lipofection to introduce exogenous genes into specific organs in vivo has certain practical advantages. Liposomes can be used to direct transfection to particular cell types, which is particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

Electroporation is another alternative mode of administration. For example, Aihara and Miyazaki, Nature Biotechnology 16:867 (1998), have demonstrated the use of in vivo electroporation for gene transfer into muscle.

In general, the dosage of a composition comprising a therapeutic vector encoding a chimeric polypeptide of the present invention, such as a recombinant virus, will vary depending upon such factors as the subject's age, weight, height, sex, general medical condition and previous medical history. Suitable routes of administration of therapeutic vectors include intravenous injection, intraarterial injection, intraperitoneal injection, intramuscular injection, intratumoral injection, and injection into a cavity that contains a tumor.

It is apparent that many modifications and variations of this invention as hereinabove set forth may be made without departing from the spirit and scope thereof. The specific embodiments are given by way of example only and the invention is limited only by the terms of the appended claims. All publications cited herein are incorporated by reference in their entireties.

### REFERENCES

Kotenko, S. V., Izotova, L. S., Pollack, B. P., Muthukumaran, G., Paukku, K., Silvennoinen, O., Ihle, J. N., and Pestka, S. (1996) Jak Kinases Can Substitute for Jak2 in the IFN-γ Signal Transduction. J. Biol. Chem., 271, 17174-17182
Kotenko, S. V., Krause, C. D., Izotova, L. S., Pollack, B. P., Wu, W., and Pestka, S. (1997) Identification and Functional Characterization of a Second Chain of the Interleukin-10 Receptor Complex. EMBO J. 16, 5894-5903
Kotenko, S. V., Izotova, L. S., Mirochnitchenko, O. V., Lee, C., Pestka, S. (1999) The Intracellular Domain of IFN-αR2c Chain is Responsible for Stat Activation. Proc. Natl. Acad. Sci. USA 96, 5007-5012
Kotenko, S. V. and Pestka, S. (2000) Jak-Stat Signal Transduction Pathway through the Eyes of Cytokine Class II Receptor Complexes. Oncogene 19, 2557-2565
Kotenko, S. V., Izotova, L. S., Mirochnitchenko, O. V., Esterova, E., Dickensheets, H., Donnelly, R. P., and Pestka, S. (2001) Identification of the Functional Interleukin-22 (IL-22) Receptor Complex: the IL-10R2 Chain (IL-10Rβ) Is a Common Chain of Both the IL-10 and IL-22 (IL-10-Related T Cell-Derived Inducible Factor, IL-TIF) Receptor Complexes. J. Biol. Chem. 276, 2725-2732
Kotenko, S. V. (2002) The Family of IL-10-Related Cytokines and Their Receptors: Related, But to What Extent? Cytokine Growth Factor Rev. 13, 223-240
Kotenko, S. V., Gallagher, G., Baurin, V. V., Lewis-Antes, A., Shen, M., Shah, N. K., Langer, J. A., Sheikh, F., Dickensheets, H., and Donnelly, R. P. (2003) Interferon-λs mediate antiviral protection through a unique class II cytokine receptor complex. Nat. Immunol. 4, 69-77
Kotenko, S. V. and Langer, J. A. (2004) Full House: Twelve Receptors for Thirty Six Cytokines. Intern. Immunopharmacol. 4, 593-608
Bromberg, J. F., Wrzeszczynska, M. H., Devgan, G., Zhao, Y., Pestell, R. G., Albanese, C., and Darnell, J. E. Jr. (1999) Stat3 as an oncogene. Cell 98, 295-303

1. A chimeric polypeptide comprising:
a first portion comprising a receptor domain, wherein the receptor domain comprises an intracellular region and a transmembrane region; and
a second portion comprising a dimerization domain.

2-10. (canceled)

11. A cell comprising the chimeric polypeptide according to claim 1.

12. A composition comprising a chimeric polypeptide according to claim 1.

13. A composition comprising:
a first chimeric polypeptide wherein the first chimeric polypeptide is a chimeric polypeptide according to claim 1; and
a second chimeric polypeptide wherein the second chimeric polypeptide is a chimeric polypeptide according to claim 1, wherein the receptor domain of the second chimeric polypeptide is derived from a receptor the same as the receptor domain of the first chimeric polypeptide;
wherein the dimerization domain of the first chimeric polypeptide binds to the dimerization domain of the second chimeric polypeptide resulting in the receptor domain of the first chimeric polypeptide associating with the receptor domain of the second chimeric polypeptide.

14-16. (canceled)

17. A polynucleotide encoding a chimeric polypeptide according to claim 1.

18-26. (canceled)

27. A method of activating a signaling pathway in a cell comprising administering to the cell a composition according to claim 13.

28-30. (canceled)

31. A method of preventing, treating, or ameliorating a disease related to increased or extended STAT3 activity in a subject comprising
administering to the subject a composition according to claim 13 wherein the composition activates a STAT1 signaling pathway in the subject.

32. A method of preventing, treating, or ameliorating a disease related to increased or extended signaling pathway induced by cytokines or growth factors in a subject comprising
administering to the subject a composition according to claim 13 wherein the composition activates a signaling pathway that counterbalances, suppresses or alters the increased or extended signaling pathway in the subject.

33. (canceled)

34. A chimeric polypeptide comprising:
a first portion comprising a receptor domain, wherein the receptor domain comprises a receptor extracellular region; and
a second portion comprising a dimerization domain, wherein the dimerization domain comprises an antibody heavy chain region of a Fab fragment or an antibody light chain region.

35-39. (canceled)

40. A cell comprising the chimeric polypeptide according to claim 34.

41. A composition comprising a chimeric polypeptide according to claim 34.

42. A composition comprising:
a first chimeric polypeptide wherein the first chimeric polypeptide is the chimeric polypeptide according to claim 34; and
a second chimeric polypeptide wherein the second chimeric polypeptide is the chimeric polypeptide according to claim 34, wherein the receptor domain of the second chimeric polypeptide is derived from a receptor different from the receptor domain of the first chimeric polypeptide;
wherein the dimerization domain of the first chimeric polypeptide binds to the dimerization domain of the second chimeric polypeptide resulting in the receptor domain of the first chimeric polypeptide associating with the receptor domain of the second chimeric polypeptide.

43. A polynucleotide encoding a chimeric polypeptide according to claim 34.

44-46. (canceled)

47. A vector comprising the polynucleotide sequence according to 43.

48. (canceled)

49. A cell comprising the polynucleotide according to claim 43.

50. A cell comprising the vector according to claim 47.

51. A composition comprising the polynucleotide according to 43.

52. A composition comprising the vector according to claim 47.

53. A method of inhibiting or reducing activation of a signaling pathway in a cell comprising administering to the cell a composition of claim 42.

54-55. (canceled)


SEQUENCE LISTING (125 sequences)

SEQ ID NO: 1 - protein, 484 aa, Artificial Sequence
  Nucleotide and amino acid sequences of chimeric antibody-receptor constructs, mH2/ gamma R1, full length
MEWSWVFLFILSGTASVHSQVQLQQPGAELARPGASVKMSCKASGYSFTSYGISWVKQKTRQGLEWIGEIYPGSGNTYYNEKFKGKATLTADKSSSTAYMQLSSLTSEDSAVYFCARWGEPWDVDYWGQGTTITVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGKWLAPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 2 - protein, 327 aa, Artificial Sequence
  Nucleotide and amino acid sequences of chimeric antibody-receptor constructs, mH2/ gamma R2, full length
MDFQVQIFSFLLISASVIISRGQIVLTQSPAIMSASPGERVTMTCSASSSVSHMHWYQQKSGTSPKRWIYDTFKLTSGVPDRFSGSGSGTSYSLTISNMEAEDVATYYCQQWSRNPPTFGVGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECQQWLAISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 3 - protein, 495 aa, Artificial Sequence
  Nucleotide and amino acid sequences of chimeric antibody-receptor constructs, SPd6xHis-hH2/gammaR1, full length
MKLCILLAVVAFVGLSLGRPHHHHHHPDPQVQLVQSGAEVKKPGESLKISCQGSGYKFTSYWIGWVRQMPGKGLEWMAIVYPSDSDARYSPSFQGRVTISADKSTSTAYMQWSALNTSDTAIYFCARGGWRDAFDVWGQGTIVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKWLAPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 4 - protein, 342 aa, Artificial Sequence
  Nucleotide and amino acid sequences of chimeric antibody-receptor constructs SPFL-hL2/gammaR2
MRPTLLWSLLLLLGVFAAAAADYKDDDDKADPVMTQSPDSLAVSLGERATINCKSSQSVVSSSDNRYYLAWYQQKPGQPPKLLIYWASTRESGVPDRFSGSGSGTDFTLTISSLQAEDMAVYYCQQYYSPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQQWLAISVGTFSLLSVLAGACFFLVLKYRGLIKYWFHTPPSIPLQIEEYLVWDSVSIISFPEKEQEDVLQKDPTQPILEALDKDSSPKDDTL

SEQ ID NO: 5 - protein, 497 aa, Artificial Sequence
  Nucleotide and amino acid sequences of chimeric antibody-receptor constructs, SPFL-mFc/gammaR1-full length
MRPTLLWSLLLLLGVFAAAAADYKDDDDKADPGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGKWLAPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 6 - protein, 548 aa, Artificial Sequence
  Nucleotide and amino acid sequences of chimeric antibody-receptor constructs, SPFL-mFc/IFN-lamdaR1-full length
MRPTLLWSLLLLLGVFAAAAADYKDDDDKADPGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGKWLALVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 7 - protein, 526 aa, Artificial Sequence
  Nucleotide and amino acid sequences of chimeric antibody-receptor constructs, SPFL-mFc/IFN-alphaR2-full length
MRPTLLWSLLLLLGVFAAAAADYKDDDDKADPGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGKWLAIITVFLIALVLTSTIVTLKWIGYICLRNSLPKVLNFHNFLAWPFPNLPPLEAMDMVEVIYINRKKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 8 - protein, 557 aa, Artificial Sequence
  Nucleotide and amino acid sequences of chimeric antibody-receptor constructs, SPFL-mFc/IL-20R1-full length
MRPTLLWSLLLLLGVFAAAAADYKDDDDKADPGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGKWLAWYVLPISITVFLFSVMGYSIYRYIHVGKEKHPANLILIYGNEFDKRFFVPAEKIVINFITLNISDDSKISHQDMSLLGKSSDVSSLNDPQPSGNLRPPQEEEEVKHLGYASHLMEIFCDSEENTEGTSLTQQESLSRTIPPDKTVIEYEYDVRTTDICAGPEEQELSLQEEVSTQGTLLESQAALAVLGPQTLQYSYTPQLQDLDPLAQEHTDSEEGPEEEPSTTLVDWDPQTGRLCIPSLSSFDQDSEGCEPSEGDGLGEEGLLSRLYEEPAPDRPPGENETYLMQFMEEWGLYVQMEN

SEQ ID NO: 9 - protein, 600 aa, Artificial Sequence
  Nucleotide and amino acid sequences of chimeric antibody-receptor constructs, SPFL-mFc/IL-20R1-full length
MRPTLLWSLLLLLGVFAAAAADYKDDDDKADPGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGKWLASGAFLFSMGFLVAVLCYLSYRYVTKPPAPPNSLNVQRVLTFQPLRFIQEHVLIPVFDLSGPSSLAQPVQYSQIRVSGPREPAGAPQRHSLSEITYLGQPDISILQPSNVPPPQILSPLSYAPNAAPEVGPPSYAPQVTPEAQFPFYAPQAISKVQPSSYAPQATPDSWPPSYGVCMEGSGKDSPTGTLSSPKHLRPKGQLQKEPPAGSCMLGGLSLQEVTSLAMEESQEAKSLHQPLGICTDRTSDPNVLHSGEEGTPQYLKGQLPLLSSVQIEGHPMSLPLQPPSGPCSPSDQGPSPWGLLESLVCPKDEAKSPAPETSDLEQPTELDSLFRGLALTVQWES

SEQ ID NO: 10 - protein, 818 aa, Artificial Sequence
  Nucleotide and amino acid sequences of chimeric antibody-receptor constructs, SPFL-mFc/EGFR-full length
MRPTLLWSLLLLLGVFAAAAADYKDDDDKADPGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGKWLAMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 11 - protein, 479 aa, Artificial Sequence
  Nucleotide and amino acid sequences of chimeric antibody-receptor constructs, IL-10R2-2xlinker-L2-linker-FL-full length
MAWSLGSWLGGCLLVSALGMVPPPENVRMNSVNFKNILQWESPAFAEGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSASGSSGGSSGTSGSSGGSSGTSTDPVMSQSPSSLPVSVGEKVTLSCKSSQSLLYSGNQKNYLAWYQQKPGQSPKLLIYWASARESGVPDRFTGSGSGTDFTLSISSVKTEDLAVYYCQQYYSYPLTFGAGTKLVLKRAAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSSGGTSPDYKDDDDKL

SEQ ID NO: 12 - protein, 496 aa, Artificial Sequence
  Nucleotide and amino acid sequences of chimeric antibody-receptor constructs IL-10R1-2xlinker-H2-linker-6xHis-full length
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNASGSSGGSSGTSGSSGGSSGTSTDPQVQLQQSDAELVKPGASVKISCKASGYTFTDHAIHWVKQNPEQGLEWIGYFSPGNDDFKYNERFKGKATLTADKSSSTAYVQLNSLTSEDSAVYFCTRSLNMAYWGQGTSVTVSSAKTTGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCTSGSSGGSSGTSHHHHHH

SEQ ID NO: 13 - protein, 705 aa, Artificial Sequence
  Nucleotide and amino acid sequences of chimeric antibody-receptor constructs IL-10R1-2xlinker-H2-Fc-full length
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNASGSSGGSSGTSGSSGGSSGTSTDPQVQLQQSDAELVKPGASVKISCKASGYTFTDHAIHWVKQNPEQGLEWIGYFSPGNDDFKYNERFKGKATLTADKSSSTAYVQLNSLTSEDSAVYFCTRSLNMAYWGQGTSVTVSSAKTTGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 686 aa, Artificial Sequence
  Nucleotide and amino acid sequences of chimeric antibody-receptor constructs, IFN-lamdaR1-linker-H2-Fc-full length
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANASGSSGGSSGTSTDPQVQLQQSDAELVKPGASVKISCKASGYTFTDHAIHWVKQNPEQGLEWIGYFSPGNDDFKYNERFKGKATLTADKSSSTAYVQLNSLTSEDSAVYFCTRSLNMAYWGQGTSVTVSSAKTTGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 698 aa, Artificial Sequence
  Nucleotide and amino acid sequences of chimeric antibody-receptor constructs, IL-22R1-2xlinker-H2-Fc-full length
MRTLLTILTVGSLAAHAPEDPSDLLQHVKFQSSNFENILTWDSGPEGTPDTVYSIEYKTYGERDWVAKKGCQRITRKSCNLTVETGNLTELYYARVTAVSAGGRSATKMTDRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRTWTASGSSGGSSGTSGSSGGSSGTSTDPQVQLQQSDAELVKPGASVKISCKASGYTFTDHAIHWVKQNPEQGLEWIGYFSPGNDDFKYNERFKGKATLTADKSSSTAYVQLNSLTSEDSAVYFCTRSLNMAYWGQGTSVTVSSAKTTGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 241 aa, Artificial Sequence
  Nucleotide and amino acid sequences of chimeric antibody-receptor constructs, mH2/ gamma R1- Ab portion
MEWSWVFLFILSGTASVHSQVQLQQPGAELARPGASVKMSCKASGYSFTSYGISWVKQKTRQGLEWIGEIYPGSGNTYYNEKFKGKATLTADKSSSTAYMQLSSLTSEDSAVYFCARWGEPWDVDYWGQGTTITVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGK

SEQ ID NO: 17 - protein, 236 aa, Artificial Sequence
  Nucleotide and amino acid sequences of chimeric antibody-receptor constructs, mH2/ gamma R2 - Ab portion
MDFQVQIFSFLLISASVIISRGQIVLTQSPAIMSASPGERVTMTCSASSSVSHMHWYQQKSGTSPKRWIYDTFKLTSGVPDRFSGSGSGTSYSLTISNMEAEDVATYYCQQWSRNPPTFGVGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECQ

SEQ ID NO: 18 - protein, 226 aa, Artificial Sequence
  Nucleotide and amino acid sequences of chimeric antibody-receptor constructs, SPd6xHis-hH2/gammaR1 - Ab portion
PDPQVQLVQSGAEVKKPGESLKISCQGSGYKFTSYWIGWVRQMPGKGLEWMAIVYPSDSDARYSPSFQGRVTISADKSTSTAYMQWSALNTSDTAIYFCARGGWRDAFDVWGQGTIVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 19 - protein, 225 aa, Artificial Sequence
  Nucleotide and amino acid sequences of chimeric antibody-receptor constructs, SPFL-hL2/gammaR2 - Ab portion
DKADPVMTQSPDSLAVSLGERATINCKSSQSVVSSSDNRYYLAWYQQKPGQPPKLLIYWASTRESGVPDRFSGSGSGTDFTLTISSLQAEDMAVYYCQQYYSPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQQ

SEQ ID NO: 20 - protein, 227 aa, Artificial Sequence
  Nucleotide and amino acid sequences of chimeric antibody-receptor constructs, SPFL-mFc/gammaR1 - Ab portion
DKADPGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 21 - protein, 225 aa, Artificial Sequence
  Nucleotide and amino acid sequences of chimeric antibody-receptor constructs, SPFL-mFc/IFN-lamdaR1 - Ab portion
ADPGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 22 - protein, 225 aa, Artificial Sequence; identical to SEQ ID NO: 21
  Nucleotide and amino acid sequences of chimeric antibody-receptor constructs, SPFL-mFc/IFN-alphaR2 - Ab portion

SEQ ID NO: 23 - protein, 225 aa, Artificial Sequence; identical to SEQ ID NO: 21
  Nucleotide and amino acid sequences of chimeric antibody-receptor constructs, SPFL-mFc/IL-20R1 - Ab portion

SEQ ID NO: 24 - protein, 225 aa, Artificial Sequence; identical to SEQ ID NO: 21
  Nucleotide and amino acid sequences of chimeric antibody-receptor constructs, SPFL-mFc/IL-20R1 - Ab portion

SEQ ID NO: 25 - protein, 225 aa, Artificial Sequence; identical to SEQ ID NO: 21
  Nucleotide and amino acid sequences of chimeric antibody-receptor constructs, SPFL-mFc/EGFR - Ab portion

SEQ ID NO: 26 - protein, 218 aa, Artificial Sequence
  Nucleotide and amino acid sequences of chimeric antibody-receptor constructs, IL-10R2-2xlinker-L2-linker-FL - Ab portion
VMSQSPSSLPVSVGEKVTLSCKSSQSLLYSGNQKNYLAWYQQKPGQSPKLLIYWASARESGVPDRFTGSGSGTDFTLSISSVKTEDLAVYYCQQYYSYPLTFGAGTKLVLKRAAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 218 aa, Artificial Sequence
  Nucleotide and amino acid sequences of chimeric antibody-receptor constructs IL-10R1-2xlinker-H2-linker-6xHis - Ab portion
QVQLQQSDAELVKPGASVKISCKASGYTFTDHAIHWVKQNPEQGLEWIGYFSPGNDDFKYNERFKGKATLTADKSSSTAYVQLNSLTSEDSAVYFCTRSLNMAYWGQGTSVTVSSAKTTGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 28 - protein, 445 aa, Artificial Sequence
  Nucleotide and amino acid sequences of chimeric antibody-receptor constructs IL-10R1-2xlinker-H2-Fc - Ab portion
QVQLQQSDAELVKPGASVKISCKASGYTFTDHAIHWVKQNPEQGLEWIGYFSPGNDDFKYNERFKGKATLTADKSSSTAYVQLNSLTSEDSAVYFCTRSLNMAYWGQGTSVTVSSAKTTGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 145 aa, Artificial Sequence
  Nucleotide and amino acid sequences of chimeric antibody-receptor constructs, IFN-lamdaR1-linker-H2-Fc - Ab portion
QVQLQQSDAELVKPGASVKISCKASGYTFTDHAIHWVKQNPEQGLEWIGYFSPGNDDFKYNERFKGKATLTADKSSSTAYVQLNSLTSEDSAVYFCTRSLNMAYWGQGTSVTVSSAKTTGPSVFPLAPSSKSTSGGTAALGCLVK

SEQ ID NO: 30 - protein, 445 aa, Artificial Sequence; identical to SEQ ID NO: 28
  Nucleotide and amino acid sequences of chimeric antibody-receptor constructs, IL-22R1-2xlinker-H2-Fc - Extracellular

SEQ ID NO: 31 - protein, 24 aa, Artificial Sequence
  Nucleotide and amino acid sequences of chimeric antibody-receptor constructs, mH2/ gamma R1- Transmembrane
WLAPVVAALLLFLVLSLVFICFYI

SEQ ID NO: 32 - protein, 25 aa, Artificial Sequence
  Nucleotide and amino acid sequences of chimeric antibody-receptor constructs, mH2/ gamma R2 - Transmembrane
QWLAISVGTFSLLSVLAGACFFLVL

SEQ ID NO: 33 - protein, 24 aa, Artificial Sequence
  Nucleotide and amino acid sequences of chimeric antibody-receptor constructs, SPd6xHis-hH2/gammaR1-Transmembrane
WLAPVVAALLLFLVLSLVFICFYI

SEQ ID NO: 34 - protein, 24 aa, Artificial Sequence
  Nucleotide and amino acid sequences of chimeric antibody-receptor constructs, SPFL-hL2/gammaR2 - Transmembrane
WLAISVGTFSLLSVLAGACFFLVL

SEQ ID NO: 35 - protein, 24 aa, Artificial Sequence
  Nucleotide and amino acid sequences of chimeric antibody-receptor constructs, SPFL-mFc/gammaR1- Transmembrane
WLAPVVAALLLFLVLSLVFICFYI

SEQ ID NO: 36 - protein, 23 aa, Artificial Sequence
  Nucleotide and amino acid sequences of chimeric antibody-receptor constructs, SPFL-mFc/IFN-lamdaR1 - Transmembrane
WLALVLPSLLILLLVIAAGGVIW

SEQ ID NO: 37 - protein, 21 aa, Artificial Sequence
  Nucleotide and amino acid sequences of chimeric antibody-receptor constructs, SPFL-mFc/IFN-alphaR2 - Transmembrane
WLAIITVFLIALVLTSTIVTL

SEQ ID NO: 38 - protein, 23 aa, Artificial Sequence
  Nucleotide and amino acid sequences of chimeric antibody-receptor constructs, SPFL-mFc/IL-20R1 - Transmembrane
WLASGAFLFSMGFLVAVLCYLSY

SEQ ID NO: 39 - protein, 23 aa, Artificial Sequence
  Nucleotide and amino acid sequences of chimeric antibody-receptor constructs, SPFL-mFc/IL-20R1 - Transmembrane
WLASGAFLFSMGFLVAVLCYLSY

SEQ ID NO: 40 - protein, 22 aa, Artificial Sequence
  Nucleotide and amino acid sequences of chimeric antibody-receptor constructs, SPFL-mFc/EGFR - Transmembrane
WLAMVGALLLLLVVALGIGLFM

SEQ ID NO: 41 - protein, 219 aa, Artificial Sequence
  Nucleotide and amino acid sequences of chimeric antibody-receptor constructs, mH2/ gamma R1 - Intracellular
KKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 42 - protein, 65 aa, Artificial Sequence
  Nucleotide and amino acid sequences of chimeric antibody-receptor constructs, mH2/ gamma R2 - Intracellular
YRGLIKYWFHTPPSIPLQIEEYLKDPTQPILEALDKDSSPKDDVWDSVSIISFPEKEQEDVLQTL

SEQ ID NO: 43 - protein, 219 aa, Artificial Sequence; identical to SEQ ID NO: 41
  Nucleotide and amino acid sequences of chimeric antibody-receptor constructs, SPd6xHis-hH2/gammaR1 - Intracellular

SEQ ID NO: 44 - protein, 66 aa, Artificial Sequence
  Nucleotide and amino acid sequences of chimeric antibody-receptor constructs, SPFL-hL2/gammaR2 - Intracellular
KYRGLIKYWFHTPPSIPLQIEEYLVWDSVSIISFPEKEQEDVLQKDPTQPILEALDKDSSPKDDTL

SEQ ID NO: 45 - protein, 219 aa, Artificial Sequence; identical to SEQ ID NO: 41
  Nucleotide and amino acid sequences of chimeric antibody-receptor constructs, SPFL-mFc/gammaR1 - Intracellular

SEQ ID NO: 46 - protein, 271 aa, Artificial Sequence
  Nucleotide and amino acid sequences of chimeric antibody-receptor constructs, SPFL-mFc/IFN-lamdaR1 - Intracellular
KTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 47 - protein, 251 aa, Artificial Sequence
  Nucleotide and amino acid sequences of chimeric antibody-receptor constructs, SPFL-mFc/IFN-alphaR2 - Intracellular
KWIGYICLRNSLPKVLNFHNFLAWPFPNLPPLEAMDMVEVIYINRKKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 48 - protein, 279 aa, Artificial Sequence
  Nucleotide and amino acid sequences of chimeric antibody-receptor constructs, SPFL-mFc/IL-20R1 - Intracellular
RYIHVGKEKHPANLILIYGNEFDKRFFVPAEKIVINFITLNISDDSKISHQDMSLLGKSSDVSSLNDPQPSGNLRPPQEEEEVKHLGYASHLMEIFCDSEENTEGTSLTQQESLSRTIPPDKTVIEYEYDVRTTDICAGPEEQELSLQEEVSTQGTLLESQAALAVLGPQTLQYSYTPQLQDLDPLAQEHTDSEEGPEEEPSTTLVDWDPQTGRLCIPSLSSFDQDSEGCEPSEGDGLGEEGLLSRLYEEPAPDRPPGENETYLMQFMEEWGLYVQMEN

SEQ ID NO: 49 - protein, 323 aa, Artificial Sequence
  Nucleotide and amino acid sequences of chimeric antibody-receptor constructs, SPFL-mFc/IL-20R1 - Intracellular
RYVTKPPAPPNSLNVQRVLTFQPLRFIQEHVLIPVFDLSGPSSLAQPVQYSQIRVSGPREPAGAPQRHSLSEITYLGQPDISILQPSNVPPPQILSPLSYAPNAAPEVGPPSYAPQVTPEAQFPFYAPQAISKVQPSSYAPQATPDSWPPSYGVCMEGSGKDSPTGTLSSPKHLRPKGQLQKEPPAGSCMLGGLSLQEVTSLAMEESQEAKSLHQPLGICTDRTSDPNVLHSGEEGTPQYLKGQLPLLSSVQIEGHPMSLPLQPPSGPCSPSDQGPSPWGLLESLVCPKDEAKSPAPETSDLEQPTELDSLFRGLALTVQWES

SEQ ID NO: 50 - protein, 542 aa, Artificial Sequence
  Nucleotide and amino acid sequences of chimeric antibody-receptor constructs, SPFL-mFc/EGFR - Intracellular
RRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 51 - protein, 220 aa, Artificial Sequence
  Nucleotide and amino acid sequences of chimeric antibody-receptor constructs, IL-10R2-2xlinker-L2-linker-FL - Extracellular
MAWSLGSWLGGCLLVSALGMVPPPENVRMNSVNFKNILQWESPAFAEGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPS

SEQ ID NO: 52 - protein, 235 aa, Artificial Sequence
  Nucleotide and amino acid sequences of chimeric antibody-receptor constructs IL-10R1-2xlinker-H2-linker-6xHis - Extracellular
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 53 - protein, 235 aa, Artificial Sequence; identical to SEQ ID NO: 52
  Nucleotide and amino acid sequences of chimeric antibody-receptor constructs IL-10R1-2xlinker-H2-Fc - Extracellular

SEQ ID NO: 54 - protein, 226 aa, Artificial Sequence
  Nucleotide and amino acid sequences of chimeric antibody-receptor constructs, IFN-lamdaR1-linker-H2-Fc - Extracellular
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEAN

SEQ ID NO: 55 - protein, 228 aa, Artificial Sequence
  Nucleotide and amino acid sequences of chimeric antibody-receptor constructs, IL-22R1-2xlinker-H2-Fc - Extracellular
MRTLLTILTVGSLAAHAPEDPSDLLQHVKFQSSNFENILTWDSGPEGTPDTVYSIEYKTYGERDWVAKKGCQRITRKSCNLTVETGNLTELYYARVTAVSAGGRSATKMTDRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRTWT

SEQ ID NO: 56 - DNA, 1452 bp, Artificial Sequence
  Nucleotide and amino acid sequences of chimeric antibody-receptor constructs, mH2/ gamma R1 - full length
atggaatggagctgggtctttctcttcatcctctcaggaactgcaagtgtccactcccaggttcagctgcagcagcctggagctgagctggcgaggcctggggcttcagtgaagatgtcctgcaaggcttctggttactcattcacaagctatggtataagctgggtgaagcagaaaactagacagggccttgagtggattggagagatttatcctggaagtggtaacacttactacaatgaaaagttcaagggcaaggccacactgactgcagacaaatcctccagcacagcctacatgcagctcagcagcctgacatctgaggactctgcagtctatttctgtgcaagatggggggagccctgggacgtggactactggggccaaggcaccactatcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggtaagtggctagcgccagttgttgctgctttactactctttctagtgcttagcctggtattcatctgtttttatattaagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttaaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttca

SEQ ID NO: 57 - DNA, 981 bp, Artificial Sequence
  Nucleotide and amino acid sequences of chimeric antibody-receptor constructs, mH2/ gamma R2 - full length
atggattttcaagtgcagattttcagcttcctgctaatcagtgcctcagtcataatatccagaggacaaattgttctcacccagtctccggcaatcatgtctgcatctccaggggagagggtcaccatgacctgcagtgccagctcaagtgtaagtcacatgcactggtaccagcagaagtcaggcacctcccccaaaagatggatttatgacacattcaagttgacttctggagtccctgatcgcttcagtggcagtgggtctgggacctcttactctctcacaatcagcaacatggaggctgaagatgttgccacttattactgccagcagtggagtagaaacccacccacgttcggtgttgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtcagcagtggctagcgatctccgtgggaacattttcgttgctgtcggtgctggcaggagcctgtttcttcctggtcctgaaatatagaggcctgattaaatactggtttcacactccaccaagcatcccattacagatagaagagtatttaaaagacccaactcagcccatcttagaggccttggacaaggacagctcaccaaaggatgacgtctgggactctgtgtccattatctcgtttccggaaaaggagcaagaagatgttctccaaacgctt

SEQ ID NO: 58 - DNA, 1485 bp, Artificial Sequence
  Nucleotide and amino acid sequences of chimeric antibody-receptor constructs, SPd6xHis-hH2/gammaR1 - full length
atgaagttatgcatattactggccgtcgtggcctttgttggcctctcgctcgggagacctcatcatcaccatcaccatccggatccccaggtgcagctggtgcagtctggggcagaggtgaaaaagcccggggagtctctgaagatctcctgtcagggttctggctacaagtttaccagttattggatcggctgggtgcgccagatgcccgggaaaggcctggagtggatggcgatcgtctatccttctgactctgatgccagatatagcccgtccttccaaggccgggtcaccatctcagccgacaagtccaccagcaccgcctacatgcagtggagcgccctgaatacctcggacaccgccatttatttctgtgcgcgaggcggctggcgtgacgcttttgatgtctggggccaagggacaattgtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaatggctagcgccagttgttgctgctttactactctttctagtgcttagcctggtattcatctgtttttatattaagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttgaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttca

SEQ ID NO: 59 - DNA, 1026 bp, Artificial Sequence
  Nucleotide and amino acid sequences of chimeric antibody-receptor constructs, SPFL-hL2/gammaR2 - full length
atgcgaccgacgctgctgtggtcgctgctgctgctgctcggagtcttcgccgccgccgccgccgactacaaggacgacgatgacaaggcggatcccgtgatgacccagtctccagactccctggctgtgtctctgggcgagagggccaccatcaactgcaagtccagccagagtgttgtatccagctccgacaataggtactacttagcttggtaccagcagaaaccaggacagcctcctaagctgctcatttactgggcatctacccgggaatccggggtccctgaccgattcagtggcagcgggtctgggacagatttcactctcaccatcagcagcctacaggctgaagatatggcagtttattactgtcagcaatattatagtcctccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtcagcagtggctagcgatctccgtgggaacattttcgttgctgtcggtgctggcaggagcctgtttcttcctggtcctgaaatatagaggcctgattaaatactggtttcacactccaccaagcatcccattacagatagaagagtatttaaaagacccaactcagcccatcttagaggccttggacaaggacagctcaccaaaggatgacgtctgggactctgtgtccattatctcgtttccggaaaaggagcaagaagatgttctccaaacgctt

SEQ ID NO: 60 - DNA, 1491 bp, Artificial Sequence
  Nucleotide and amino acid sequences of chimeric antibody-receptor constructs, SPFL-mFc/gammaR1 - full length
atgcgaccgacgctgctgtggtcgctgctgctgctgctcggagtcttcgccgccgccgccgccgactacaaggacgacgatgacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatggctagcgccagttgttgctgctttactactctttctagtgcttagcctggtattcatctgtttttatattaagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttaaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttca

SEQ ID NO: 61 - DNA, 1644 bp, Artificial Sequence
  Nucleotide and amino acid sequences of chimeric antibody-receptor constructs, SPFL-mFc/IFN-lamdaR1 - full length
atgcgaccgacgctgctgtggtcgctgctgctgctgctcggagtcttcgccgccgccgccgccgactacaaggacgacgatgacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatggctagccctggtgctgccatcgcttctgatactgctgttagtaattgccgcagggggtgtgatctggaagaccctcatggggaacccctggtttcagcgggcaaagatgccacgggccctggacttttctggacacacacaccctgtggcaacctttcagcccagcagaccagagtccgtgaatgacttgttcctctgtccccaaaaggaactgaccagaggggtcaggccgacgcctcgagtcagggccccagccacccaacagacaagatggaagaaggaccttgcagaggacgaagaggaggaggatgaggaggacacagaagatggcgtcagcttccagccctacattgaaccaccttctttcctggggcaagagcaccaggctccagggcactcggaggctggtggggtggactcagggaggcccagggctcctctggtcccaagcgaaggctcctctgcttgggattcttcagacagaagctgggccagcactgtggactcctcctgggacagggctgggtcctctggctatttggctgagaaggggccaggccaagggccgggtggggatgggcaccaagaatctctcccaccacctgaattctccaaggactcgggtttcctggaagagctcccagaagataacctctcctcctgggccacctggggcaccttaccaccggagccgaatctggtccctgggggacccccagtttctcttcagacactgaccttctgctgggaaagcagccctgaggaggaagaggaggcgagggaatcagaaattgaggacagcgatgcgggcagctggggggctgagagcacccagaggaccgaggacaggggccggacattggggcattacatggccagg

SEQ ID NO: 62 - DNA, 1578 bp, Artificial Sequence
  Nucleotide and amino acid sequences of chimeric antibody-receptor constructs, SPFL-mFc/IFN-alphaR2 - full length
atgcgaccgacgctgctgtggtcgctgctgctgctgctcggagtcttcgccgccgccgccgccgactacaaggacgacgatgacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatggctagcaataattactgtgtttttgatagcattggtcttgacaagcaccatagtgacactgaaatggattggttatatatgcttaagaaatagcctccccaaagtcttgaattttcataactttttagcctggccatttcctaacctgccaccgttggaagccatggatatggtggaggtcatttacatcaacagaaagaagaaagtgtgggattataattatgatgatgaaagtgatagcgatactgaggcagcgcccaggacaagtggcggtggctataccatgcatggactgactgtcaggcctctgggtcaggcctctgccacctctacagaatcccagttgatagacccggagtccgaggaggagcctgacctgcctgaggttgatgtggagctccccacgatgccaaaggacagccctcagcagttggaactcttgagtgggccctgtgagaggagaaagagtccactccaggacccttttcccgaagaggactacagctccacggaggggtctgggggcagaattaccttcaatgtggacttaaactctgtgtttttgagagttcttgatgacgaggacagtgacgacttagaagcccctctgatgctatcgtctcatctggaagagatggttgacccagaggatcctgataatgtgcaatcaaaccatttgctggccagcggggaagggacacagccaacctttcccagcccctcttcagagggcctgtggtccgaagatgctccatctgatcaaagtgacacttctgagtcagatgttgaccttggggatggttatataatgaga

SEQ ID NO: 63 - DNA, 1671 bp, Artificial Sequence
  Nucleotide and amino acid sequences of chimeric antibody-receptor constructs, SPFL-mFc/IL-20R1 - full length
atgcgaccgacgctgctgtggtcgctgctgctgctgctcggagtcttcgccgccgccgccgccgactacaaggacgacgatgacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatggctagcctggtatgttttgcccatatctattaccgtgtttcttttttctgtgatgggctattccatctaccgatatatccacgttggcaaagagaaacacccagcaaatttgattttgatttatggaaatgaatttgacaaaagattctttgtgcctgctgaaaaaatcgtgattaactttatcaccctcaatatctcggatgattctaaaatttctcaccaggatatgagtttactgggaaaaagcagtgatgtatccagccttaatgatcctcagcccagcgggaacctgaggccccctcaggaggaagaggaggtgaaacatttagggtatgcttcgcatttgatggaaattttttgtgactctgaagaaaacacggaaggtacttctctcacccagcaagagtccctcagcagaacaatacccccggataaaacagtcattgaatatgaatatgatgtcagaaccactgacatttgtgcggggcctgaagagcaggagctcagtttgcaggaggaggtgtccacacaaggaacattattggagtcgcaggcagcgttggcagtcttgggcccgcaaacgttacagtactcatacacccctcagctccaagacttagaccccctggcgcaggagcacacagactcggaggaggggccggaggaagagccatcgacgaccctggtcgactgggatccccaaactggcaggctgtgtattccttcgctgtccagcttcgaccaggattcagagggctgcgagccttctgagggggatgggctcggagaggagggtcttctatctagactctatgaggagccggctccagacaggccaccaggagaaaatgaaacctatctcatgcaattcatggaggaatgggggttatatgtgcagatggaaaac

SEQ ID NO: 64 - DNA, 1800 bp, Artificial Sequence
  Nucleotide and amino acid sequences of chimeric antibody-receptor constructs, SPFL-mFc/IL-20R1 - full length
atgcgaccgacgctgctgtggtcgctgctgctgctgctcggagtcttcgccgccgccgccgccgactacaaggacgacgatgacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatggctagcctccggagccttcctgttctccatgggcttcctcgtcgcagtactctgctacctgagctacagatatgtcaccaagccgcctgcacctcccaactccctgaacgtccagcgagtcctgactttccagccgctgcgcttcatccaggagcacgtcctgatccctgtctttgacctcagcggccccagcagtctggcccagcctgtccagtactcccagatcagggtgtctggacccagggagcccgcaggagctccacagcggcatagcctgtccgagatcacctacttagggcagccagacatctccatcctccagccctccaacgtgccacctccccagatcctctccccactgtcctatgccccaaacgctgcccctgaggtcgggcccccatcctatgcacctcaggtgacccccgaagctcaattcccattctacgccccacaggccatctctaaggtccagccttcctcctatgcccctcaagccactccggacagctggcctccctcctatggggtatgcatggaaggttctggcaaagactcccccactgggacactttctagtcctaaacaccttaggcctaaaggtcagcttcagaaagagccaccagctggaagctgcatgttaggtggcctttctctgcaggaggtgacctccttggctatggaggaatcccaagaagcaaaatcattgcaccagcccctggggatttgcacagacagaacatctgacccaaatgtgctacacagtggggaggaagggacaccacagtacctaaagggccagctccccctcctctcctcagtccagatcgagggccaccccatgtccctccctttgcaacctccttccggtccatgttccccctcggaccaaggtccaagtccctggggcctgctggagtcccttgtgtgtcccaaggatgaagccaagagcccagcccctgagacctcagacctggagcagcccacagaactggattctcttttcagaggcctggccctgactgtgcagtgggagtcc

SEQ ID NO: 65 - DNA, 2454 bp, Artificial Sequence
  Nucleotide and amino acid sequences of chimeric antibody-receptor constructs, SPFL-mFc/EGFR, full length
atgcgaccgacgctgctgtggtcgctgctgctgctgctcggagtcttcgccgccgccgccgccgactacaaggacgacgatgacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatggctagcgatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcaactcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagca

SEQ ID NO: 66 - DNA, 1437 bp, Artificial Sequence
  Nucleotide and amino acid sequences of chimeric antibody-receptor constructs, IL-10R2-2xlinker-L2-linker-FL - full length
atggcgtggagccttgggagctggctgggtggctgcctgctggtgtcagcattgggaatggtgccacctcccgaaaatgtcagaatgaattctgttaatttcaagaacattctacagtgggagtcacctgcttttgccgaagggaacctgactttcacagctcagtacctaagttataggatattccaagataaatgcatgaatactaccttgacggaatgtgatttctcaagtctttccaagtatggtgaccacaccttgagagtcagggctgaatttgcagatgagcattcagactgggtaaacatcaccttctgtcctgtggatgacaccattattggaccccctggaatgcaagtagaagtacttgctgattctttacatatgcgtttcttagcccctaaaattgagaatgaatacgaaacttggactatgaagaatgtgtataactcatggacttataatgtgcaatactggaaaaacggtactgatgaaaagtttcaaattactccccagtatgactttgaggtcctcagaaacctggagccatggacaacttattgtgttcaagttcgagggtttcttcctgatcggaacaaagctggggaatggagtgagcctgtctgtgagcaaacaacccatgacgaaacggtcccctccgctagcgggagttctggtggtagctctgggactagcgggagttctggtggtagctctgggactagtacggatcctgtgatgtcacagtctccatcctccctacctgtgtcagttggcgagaaggttactttgagctgcaagtccagtcagagccttttatatagtggtaatcaaaagaactacttggcctggtaccagcagaaaccagggcagtctcctaaactgctgatttactgggcatccgctagggaatctggggtccctgatcgcttcacaggcagtggatctgggacagatttcactctctccatcagcagtgtgaagactgaagacctggcagtttattactgtcagcagtattatagctatcccctcacgttcggtgctgggaccaagctggtgctgaaacgggccgcggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttcgtcggggggaactagccctgactacaaggacgacgatgacaagctc

SEQ ID NO: 67 - DNA, 1488 bp, Artificial Sequence
  Nucleotide and amino acid sequences of chimeric antibody-receptor constructs IL-10R1-2xlinker-H2-linker-6xHis - full length
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgctagcgggagttctggtggtagctctgggactagcgggagttctggtggtagctctgggactagtacggatccccaggttcagttgcagcagtctgacgctgagttggtgaaacctggggcttcagtgaagatttcctgcaaggcttctggctacaccttcactgaccatgcaattcactgggtgaaacagaaccctgaacagggcctggaatggattggatatttttctcccggaaatgatgattttaaatacaatgagaggttcaagggcaaggccacactgactgcagacaaatcctccagcactgcctacgtgcagctcaacagcctgacatctgaggattctgcagtgtatttctgtacaagatccctgaatatggcctactggggtcaaggaacctcagtcaccgtctcctcagccaaaacgacgggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtactagcgggagttctggtggtagctctgggactagccatcatcaccatcaccat

SEQ ID NO: 68 - DNA, 2115 bp, Artificial Sequence
  Nucleotide and amino acid sequences of chimeric antibody-receptor constructs IL-10R1-2xlinker-H2-Fc - full length
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgctagcgggagttctggtggtagctctgggactagcgggagttctggtggtagctctgggactagtacggatccccaggttcagttgcagcagtctgacgctgagttggtgaaacctggggcttcagtgaagatttcctgcaaggcttctggctacaccttcactgaccatgcaattcactgggtgaaacagaaccctgaacagggcctggaatggattggatatttttctcccggaaatgatgattttaaatacaatgagaggttcaagggcaaggccacactgactgcagacaaatcctccagcactgcctacgtgcagctcaacagcctgacatctgaggattctgcagtgtatttctgtacaagatccctgaatatggcctactggggtcaaggaacctcagtcaccgtctcctcagccaaaacgacgggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 69 - DNA, 2058 bp, Artificial Sequence
  Nucleotide and amino acid sequences of chimeric antibody-receptor constructs, IFN-lamdaR1-linker-H2-Fc - full length
atggcggggcccgagcgctggggccccctgctcctgtgcctgctgcaggccgctccagggaggccccgtctggcccctccccagaatgtgacgctgctctcccagaacttcagcgtgtacctgacatggctcccagggcttggcaacccccaggatgtgacctattttgtggcctatcagagctctcctacccgtagacggtggcgcgaagtggaagagtgtgcgggaaccaaggagctgctatgttctatgatgtgcctgaagaaacaggacctgtacaacaagttcaagggacgcgtgcggacggtttctcccagctccaagtccccctgggtggagtccgaatacctggattacctttttgaagtggagccggccccacctgtcctggtgctcacccagacggaggagatcctgagtgccaatgccacgtaccagctgcccccctgcatgcccccactggatctgaagtatgaggtggcattctggaaggagggggccggaaacaagaccctatttccagtcactccccatggccagccagtccagatcactctccagccagctgccagcgaacaccactgcctcagtgccagaaccatctacacgttcagtgtcccgaaatacagcaagttctctaagcccacctgcttcttgctggaggtcccagaagccaacgctagcgggagttctggtggtagctctgggactagtacggatccccaggttcagttgcagcagtctgacgctgagttggtgaaacctggggcttcagtgaagatttcctgcaaggcttctggctacaccttcactgaccatgcaattcactgggtgaaacagaaccctgaacagggcctggaatggattggatatttttctcccggaaatgatgattttaaatacaatgagaggttcaagggcaaggccacactgactgcagacaaatcctccagcactgcctacgtgcagctcaacagcctgacatctgaggattctgcagtgtatttctgtacaagatccctgaatatggcctactggggtcaaggaacctcagtcaccgtctcctcagccaaaacgacgggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 70 - DNA, 2097 bp, Artificial Sequence
  Nucleotide and amino acid sequences of chimeric antibody-receptor constructs, IL-22R1-2xlinker-H2-Fc - full length
atgaggacgctgctgaccatcttgactgtgggatccctggctgctcacgcccctgaggacccctcggatctgctccagcacgtgaaattccagtccagcaactttgaaaacatcctgacgtgggacagcgggccagagggcaccccagacacggtctacagcatcgagtataagacgtacggagagagggactgggtggcaaagaagggctgtcagcggatcacccggaagtcctgcaacctgacggtggagacgggcaacctcacggagctctactatgccagggtcaccgctgtcagtgcgggaggccggtcagccaccaagatgactgacaggttcagctctctgcagcacactaccctcaagccacctgatgtgacctgtatctccaaagtgagatcgattcagatgattgttcatcctacccccacgccaatccgtgcaggcgatggccaccggctaaccctggaagacatcttccatgacctgttctaccacttagagctccaggtcaaccgcacctaccaaatgcaccttggagggaagcagagagaatatgagttcttcggcctgacccctgacacagagttccttggcaccatcatgatttgcgttcccacctgggccaaggagagtgccccctacatgtgccgagtgaagacactgccagaccggacatggaccgctagcgggagttctggtggtagctctgggactagcgggagttctggtggtagctctgggactagtacggatccccaggttcagttgcagcagtctgacgctgagttggtgaaacctggggcttcagtgaagatttcctgcaaggcttctggctacaccttcactgaccatgcaattcactgggtgaaacagaaccctgaacagggcctggaatggattggatatttttctcccggaaatgatgattttaaatacaatgagaggttcaagggcaaggccacactgactgcagacaaatcctccagcactgcctacgtgcagctcaacagcctgacatctgaggattctgcagtgtatttctgtacaagatccctgaatatggcctactggggtcaaggaacctcagtcaccgtctcctcagccaaaacgacgggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 71 - DNA, 1455 bp, Artificial Sequence
  Nucleotide and amino acid sequences of chimeric antibody-receptor constructs, mH2/ gamma R1 - full length + stop codon
atggaatggagctgggtctttctcttcatcctctcaggaactgcaagtgtccactcccaggttcagctgcagcagcctggagctgagctggcgaggcctggggcttcagtgaagatgtcctgcaaggcttctggttactcattcacaagctatggtataagctgggtgaagcagaaaactagacagggccttgagtggattggagagatttatcctggaagtggtaacacttactacaatgaaaagttcaagggcaaggccacactgactgcagacaaatcctccagcacagcctacatgcagctcagcagcctgacatctgaggactctgcagtctatttctgtgcaagatggggggagccctgggacgtggactactggggccaaggcaccactatcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggtaagtggctagcgccagttgttgctgctttactactctttctagtgcttagcctggtattcatctgtttttatattaagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttaaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttcatga

SEQ ID NO: 72 - DNA, 984 bp, Artificial Sequence
  Nucleotide and amino acid sequences of chimeric antibody-receptor constructs, mH2/ gamma R2 - full length + stop codon
atggattttcaagtgcagattttcagcttcctgctaatcagtgcctcagtcataatatccagaggacaaattgttctcacccagtctccggcaatcatgtctgcatctccaggggagagggtcaccatgacctgcagtgccagctcaagtgtaagtcacatgcactggtaccagcagaagtcaggcacctcccccaaaagatggatttatgacacattcaagttgacttctggagtccctgatcgcttcagtggcagtgggtctgggacctcttactctctcacaatcagcaacatggaggctgaagatgttgccacttattactgccagcagtggagtagaaacccacccacgttcggtgttgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtcagcagtggctagcgatctccgtgggaacattttcgttgctgtcggtgctggcaggagcctgtttcttcctggtcctgaaatatagaggcctgattaaatactggtttcacactccaccaagcatcccattacagatagaagagtatttaaaagacccaactcagcccatcttagaggccttggacaaggacagctcaccaaaggatgacgtctgggactctgtgtccattatctcgtttccggaaaaggagcaagaagatgttctccaaacgctttga

SEQ ID NO: 73 - DNA, 1488 bp, Artificial Sequence
  Nucleotide and amino acid sequences of chimeric antibody-receptor constructs, SPd6xHis-hH2/gammaR1 - full length + stop codon
atgaagttatgcatattactggccgtcgtggcctttgttggcctctcgctcgggagacctcatcatcaccatcaccatccggatccccaggtgcagctggtgcagtctggggcagaggtgaaaaagcccggggagtctctgaagatctcctgtcagggttctggctacaagtttaccagttattggatcggctgggtgcgccagatgcccgggaaaggcctggagtggatggcgatcgtctatccttctgactctgatgccagatatagcccgtccttccaaggccgggtcaccatctcagccgacaagtccaccagcaccgcctacatgcagtggagcgccctgaatacctcggacaccgccatttatttctgtgcgcgaggcggctggcgtgacgcttttgatgtctggggccaagggacaattgtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaatggctagcgccagttgttgctgctttactactctttctagtgcttagcctggtattcatctgtttttatattaagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttgaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttcatga

SEQ ID NO: 74 - DNA, 1029 bp, Artificial Sequence
  Nucleotide and amino acid sequences of chimeric antibody-receptor constructs, SPFL-hL2/gammaR2 - full length + stop condon
atgcgaccgacgctgctgtggtcgctgctgctgctgctcggagtcttcgccgccgccgccgccgactacaaggacgacgatgacaaggcggatcccgtgatgacccagtctccagactccctggctgtgtctctgggcgagagggccaccatcaactgcaagtccagccagagtgttgtatccagctccgacaataggtactacttagcttggtaccagcagaaaccaggacagcctcctaagctgctcatttactgggcatctacccgggaatccggggtccctgaccgattcagtggcagcgggtctgggacagatttcactctcaccatcagcagcctacaggctgaagatatggcagtttattactgtcagcaatattatagtcctccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtcagcagtggctagcgatctccgtgggaacattttcgttgctgtcggtgctggcaggagcctgtttcttcctggtcctgaaatatagaggcctgattaaatactggtttcacactccaccaagcatcccattacagatagaagagtatttaaaagacccaactcagcccatcttagaggccttggacaaggacagctcaccaaaggatgacgtctgggactctgtgtccattatctcgtttccggaaaaggagcaagaagatgttctccaaacgctttga

SEQ ID NO: 75 - DNA, 1494 bp, Artificial Sequence
  Nucleotide and amino acid sequences of chimeric antibody-receptor constructs, SPFL-mFc/gammaR1 - full length + stop codon
atgcgaccgacgctgctgtggtcgctgctgctgctgctcggagtcttcgccgccgccgccgccgactacaaggacgacgatgacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatggctagcgccagttgttgctgctttactactctttctagtgcttagcctggtattcatctgtttttatattaagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttaaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttcatga

SEQ ID NO: 76 - DNA, 1647 bp, Artificial Sequence
  Nucleotide and amino acid sequences of chimeric antibody-receptor constructs, SPFL-mFc/IFN-lamdaR1 - full length + stop codon
atgcgaccgacgctgctgtggtcgctgctgctgctgctcggagtcttcgccgccgccgccgccgactacaaggacgacgatgacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatggctagccctggtgctgccatcgcttctgatactgctgttagtaattgccgcagggggtgtgatctggaagaccctcatggggaacccctggtttcagcgggcaaagatgccacgggccctggacttttctggacacacacaccctgtggcaacctttcagcccagcagaccagagtccgtgaatgacttgttcctctgtccccaaaaggaactgaccagaggggtcaggccgacgcctcgagtcagggccccagccacccaacagacaagatggaagaaggaccttgcagaggacgaagaggaggaggatgaggaggacacagaagatggcgtcagcttccagccctacattgaaccaccttctttcctggggcaagagcaccaggctccagggcactcggaggctggtggggtggactcagggaggcccagggctcctctggtcccaagcgaaggctcctctgcttgggattcttcagacagaagctgggccagcactgtggactcctcctgggacagggctgggtcctctggctatttggctgagaaggggccaggccaagggccgggtggggatgggcaccaagaatctctcccaccacctgaattctccaaggactcgggtttcctggaagagctcccagaagataacctctcctcctgggccacctggggcaccttaccaccggagccgaatctggtccctgggggacccccagtttctcttcagacactgaccttctgctgggaaagcagccctgaggaggaagaggaggcgagggaatcagaaattgaggacagcgatgcgggcagctggggggctgagagcacccagaggaccgaggacaggggccggacattggggcattacatggccaggtga

SEQ ID NO: 77 - DNA, 1581 bp, Artificial Sequence
  Nucleotide and amino acid sequences of chimeric antibody-receptor constructs, SPFL-mFc/IFN-alphaR2 - full length + stop codon
atgcgaccgacgctgctgtggtcgctgctgctgctgctcggagtcttcgccgccgccgccgccgactacaaggacgacgatgacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatggctagcaataattactgtgtttttgatagcattggtcttgacaagcaccatagtgacactgaaatggattggttatatatgcttaagaaatagcctccccaaagtcttgaattttcataactttttagcctggccatttcctaacctgccaccgttggaagccatggatatggtggaggtcatttacatcaacagaaagaagaaagtgtgggattataattatgatgatgaaagtgatagcgatactgaggcagcgcccaggacaagtggcggtggctataccatgcatggactgactgtcaggcctctgggtcaggcctctgccacctctacagaatcccagttgatagacccggagtccgaggaggagcctgacctgcctgaggttgatgtggagctccccacgatgccaaaggacagccctcagcagttggaactcttgagtgggccctgtgagaggagaaagagtccactccaggacccttttcccgaagaggactacagctccacggaggggtctgggggcagaattaccttcaatgtggacttaaactctgtgtttttgagagttcttgatgacgaggacagtgacgacttagaagcccctctgatgctatcgtctcatctggaagagatggttgacccagaggatcctgataatgtgcaatcaaaccatttgctggccagcggggaagggacacagccaacctttcccagcccctcttcagagggcctgtggtccgaagatgctccatctgatcaaagtgacacttctgagtcagatgttgaccttggggatggttatataatgagatga

SEQ ID NO: 78 - DNA, 1674 bp, Artificial Sequence
  Nucleotide and amino acid sequences of chimeric antibody-receptor constructs, SPFL-mFc/IL-20R1 - full length + stop condon
atgcgaccgacgctgctgtggtcgctgctgctgctgctcggagtcttcgccgccgccgccgccgactacaaggacgacgatgacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatggctagcctggtatgttttgcccatatctattaccgtgtttcttttttctgtgatgggctattccatctaccgatatatccacgttggcaaagagaaacacccagcaaatttgattttgatttatggaaatgaatttgacaaaagattctttgtgcctgctgaaaaaatcgtgattaactttatcaccctcaatatctcggatgattctaaaatttctcaccaggatatgagtttactgggaaaaagcagtgatgtatccagccttaatgatcctcagcccagcgggaacctgaggccccctcaggaggaagaggaggtgaaacatttagggtatgcttcgcatttgatggaaattttttgtgactctgaagaaaacacggaaggtacttctctcacccagcaagagtccctcagcagaacaatacccccggataaaacagtcattgaatatgaatatgatgtcagaaccactgacatttgtgcggggcctgaagagcaggagctcagtttgcaggaggaggtgtccacacaaggaacattattggagtcgcaggcagcgttggcagtcttgggcccgcaaacgttacagtactcatacacccctcagctccaagacttagaccccctggcgcaggagcacacagactcggaggaggggccggaggaagagccatcgacgaccctggtcgactgggatccccaaactggcaggctgtgtattccttcgctgtccagcttcgaccaggattcagagggctgcgagccttctgagggggatgggctcggagaggagggtcttctatctagactctatgaggagccggctccagacaggccaccaggagaaaatgaaacctatctcatgcaattcatggaggaatgggggttatatgtgcagatggaaaactga

SEQ ID NO: 79 - DNA, 1803 bp, Artificial Sequence
  Nucleotide and amino acid sequences of chimeric antibody-receptor constructs, SPFL-mFc/IL-20R1 - full length + stop codon
atgcgaccgacgctgctgtggtcgctgctgctgctgctcggagtcttcgccgccgccgccgccgactacaaggacgacgatgacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatggctagcctccggagccttcctgttctccatgggcttcctcgtcgcagtactctgctacctgagctacagatatgtcaccaagccgcctgcacctcccaactccctgaacgtccagcgagtcctgactttccagccgctgcgcttcatccaggagcacgtcctgatccctgtctttgacctcagcggccccagcagtctggcccagcctgtccagtactcccagatcagggtgtctggacccagggagcccgcaggagctccacagcggcatagcctgtccgagatcacctacttagggcagccagacatctccatcctccagccctccaacgtgccacctccccagatcctctccccactgtcctatgccccaaacgctgcccctgaggtcgggcccccatcctatgcacctcaggtgacccccgaagctcaattcccattctacgccccacaggccatctctaaggtccagccttcctcctatgcccctcaagccactccggacagctggcctccctcctatggggtatgcatggaaggttctggcaaagactcccccactgggacactttctagtcctaaacaccttaggcctaaaggtcagcttcagaaagagccaccagctggaagctgcatgttaggtggcctttctctgcaggaggtgacctccttggctatggaggaatcccaagaagcaaaatcattgcaccagcccctggggatttgcacagacagaacatctgacccaaatgtgctacacagtggggaggaagggacaccacagtacctaaagggccagctccccctcctctcctcagtccagatcgagggccaccccatgtccctccctttgcaacctccttccggtccatgttccccctcggaccaaggtccaagtccctggggcctgctggagtcccttgtgtgtcccaaggatgaagccaagagcccagcccctgagacctcagacctggagcagcccacagaactggattctcttttcagaggcctggccctgactgtgcagtgggagtcctga

SEQ ID NO: 80 - DNA, 2457 bp, Artificial Sequence
  Nucleotide and amino acid sequences of chimeric antibody-receptor constructs, SPFL-mFc/EGFR, full length plus stop codon
atgcgaccgacgctgctgtggtcgctgctgctgctgctcggagtcttcgccgccgccgccgccgactacaaggacgacgatgacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatggctagcgatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcaactcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 81 - DNA, 1440 bp, Artificial Sequence
  Nucleotide and amino acid sequences of chimeric antibody-receptor constructs, IL-10R2-2xlinker-L2-linker-FLm- full length + stop codon
atggcgtggagccttgggagctggctgggtggctgcctgctggtgtcagcattgggaatggtgccacctcccgaaaatgtcagaatgaattctgttaatttcaagaacattctacagtgggagtcacctgcttttgccgaagggaacctgactttcacagctcagtacctaagttataggatattccaagataaatgcatgaatactaccttgacggaatgtgatttctcaagtctttccaagtatggtgaccacaccttgagagtcagggctgaatttgcagatgagcattcagactgggtaaacatcaccttctgtcctgtggatgacaccattattggaccccctggaatgcaagtagaagtacttgctgattctttacatatgcgtttcttagcccctaaaattgagaatgaatacgaaacttggactatgaagaatgtgtataactcatggacttataatgtgcaatactggaaaaacggtactgatgaaaagtttcaaattactccccagtatgactttgaggtcctcagaaacctggagccatggacaacttattgtgttcaagttcgagggtttcttcctgatcggaacaaagctggggaatggagtgagcctgtctgtgagcaaacaacccatgacgaaacggtcccctccgctagcgggagttctggtggtagctctgggactagcgggagttctggtggtagctctgggactagtacggatcctgtgatgtcacagtctccatcctccctacctgtgtcagttggcgagaaggttactttgagctgcaagtccagtcagagccttttatatagtggtaatcaaaagaactacttggcctggtaccagcagaaaccagggcagtctcctaaactgctgatttactgggcatccgctagggaatctggggtccctgatcgcttcacaggcagtggatctgggacagatttcactctctccatcagcagtgtgaagactgaagacctggcagtttattactgtcagcagtattatagctatcccctcacgttcggtgctgggaccaagctggtgctgaaacgggccgcggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttcgtcggggggaactagccctgactacaaggacgacgatgacaagctctag

SEQ ID NO: 82 - DNA, 1491 bp, Artificial Sequence
  Nucleotide and amino acid sequences of chimeric antibody-receptor constructs IL-10R1-2xlinker-H2-linker-6xHis - full length + stop codon
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgctagcgggagttctggtggtagctctgggactagcgggagttctggtggtagctctgggactagtacggatccccaggttcagttgcagcagtctgacgctgagttggtgaaacctggggcttcagtgaagatttcctgcaaggcttctggctacaccttcactgaccatgcaattcactgggtgaaacagaaccctgaacagggcctggaatggattggatatttttctcccggaaatgatgattttaaatacaatgagaggttcaagggcaaggccacactgactgcagacaaatcctccagcactgcctacgtgcagctcaacagcctgacatctgaggattctgcagtgtatttctgtacaagatccctgaatatggcctactggggtcaaggaacctcagtcaccgtctcctcagccaaaacgacgggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtactagcgggagttctggtggtagctctgggactagccatcatcaccatcaccattga

SEQ ID NO: 83 - DNA, 2118 bp, Artificial Sequence
  Nucleotide and amino acid sequences of chimeric antibody-receptor constructs IL-10R1-2xlinker-H2-Fc - full length + stop codon
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgctagcgggagttctggtggtagctctgggactagcgggagttctggtggtagctctgggactagtacggatccccaggttcagttgcagcagtctgacgctgagttggtgaaacctggggcttcagtgaagatttcctgcaaggcttctggctacaccttcactgaccatgcaattcactgggtgaaacagaaccctgaacagggcctggaatggattggatatttttctcccggaaatgatgattttaaatacaatgagaggttcaagggcaaggccacactgactgcagacaaatcctccagcactgcctacgtgcagctcaacagcctgacatctgaggattctgcagtgtatttctgtacaagatccctgaatatggcctactggggtcaaggaacctcagtcaccgtctcctcagccaaaacgacgggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 84 - DNA, 2061 bp, Artificial Sequence
  Nucleotide and amino acid sequences of chimeric antibody-receptor constructs, IFN-lamdaR1-linker-H2-Fc - full length + stop codon
atggcggggcccgagcgctggggccccctgctcctgtgcctgctgcaggccgctccagggaggccccgtctggcccctccccagaatgtgacgctgctctcccagaacttcagcgtgtacctgacatggctcccagggcttggcaacccccaggatgtgacctattttgtggcctatcagagctctcctacccgtagacggtggcgcgaagtggaagagtgtgcgggaaccaaggagctgctatgttctatgatgtgcctgaagaaacaggacctgtacaacaagttcaagggacgcgtgcggacggtttctcccagctccaagtccccctgggtggagtccgaatacctggattacctttttgaagtggagccggccccacctgtcctggtgctcacccagacggaggagatcctgagtgccaatgccacgtaccagctgcccccctgcatgcccccactggatctgaagtatgaggtggcattctggaaggagggggccggaaacaagaccctatttccagtcactccccatggccagccagtccagatcactctccagccagctgccagcgaacaccactgcctcagtgccagaaccatctacacgttcagtgtcccgaaatacagcaagttctctaagcccacctgcttcttgctggaggtcccagaagccaacgctagcgggagttctggtggtagctctgggactagtacggatccccaggttcagttgcagcagtctgacgctgagttggtgaaacctggggcttcagtgaagatttcctgcaaggcttctggctacaccttcactgaccatgcaattcactgggtgaaacagaaccctgaacagggcctggaatggattggatatttttctcccggaaatgatgattttaaatacaatgagaggttcaagggcaaggccacactgactgcagacaaatcctccagcactgcctacgtgcagctcaacagcctgacatctgaggattctgcagtgtatttctgtacaagatccctgaatatggcctactggggtcaaggaacctcagtcaccgtctcctcagccaaaacgacgggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 85 - DNA, 2100 bp, Artificial Sequence
  Nucleotide and amino acid sequences of chimeric antibody-receptor constructs, IL-22R1-2xlinker-H2-Fc - full length + stop codon
atgaggacgctgctgaccatcttgactgtgggatccctggctgctcacgcccctgaggacccctcggatctgctccagcacgtgaaattccagtccagcaactttgaaaacatcctgacgtgggacagcgggccagagggcaccccagacacggtctacagcatcgagtataagacgtacggagagagggactgggtggcaaagaagggctgtcagcggatcacccggaagtcctgcaacctgacggtggagacgggcaacctcacggagctctactatgccagggtcaccgctgtcagtgcgggaggccggtcagccaccaagatgactgacaggttcagctctctgcagcacactaccctcaagccacctgatgtgacctgtatctccaaagtgagatcgattcagatgattgttcatcctacccccacgccaatccgtgcaggcgatggccaccggctaaccctggaagacatcttccatgacctgttctaccacttagagctccaggtcaaccgcacctaccaaatgcaccttggagggaagcagagagaatatgagttcttcggcctgacccctgacacagagttccttggcaccatcatgatttgcgttcccacctgggccaaggagagtgccccctacatgtgccgagtgaagacactgccagaccggacatggaccgctagcgggagttctggtggtagctctgggactagcgggagttctggtggtagctctgggactagtacggatccccaggttcagttgcagcagtctgacgctgagttggtgaaacctggggcttcagtgaagatttcctgcaaggcttctggctacaccttcactgaccatgcaattcactgggtgaaacagaaccctgaacagggcctggaatggattggatatttttctcccggaaatgatgattttaaatacaatgagaggttcaagggcaaggccacactgactgcagacaaatcctccagcactgcctacgtgcagctcaacagcctgacatctgaggattctgcagtgtatttctgtacaagatccctgaatatggcctactggggtcaaggaacctcagtcaccgtctcctcagccaaaacgacgggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaatt

SEQ ID NO: 86 - DNA, 723 bp, Artificial Sequence
  Nucleotide and amino acid sequences of chimeric antibody-receptor constructs, mH2/ gamma R1 - Ab portion
atggaatggagctgggtctttctcttcatcctctcaggaactgcaagtgtccactcccaggttcagctgcagcagcctggagctgagctggcgaggcctggggcttcagtgaagatgtcctgcaaggcttctggttactcattcacaagctatggtataagctgggtgaagcagaaaactagacagggccttgagtggattggagagatttatcctggaagtggtaacacttactacaatgaaaagttcaagggcaaggccacactgactgcagacaaatcctccagcacagcctacatgcagctcagcagcctgacatctgaggactctgcagtctatttctgtgcaagatggggggagccctgggacgtggactactggggccaaggcaccactatcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggtaag

SEQ ID NO: 87 - DNA, 711 bp, Artificial Sequence
  Nucleotide and amino acid sequences of chimeric antibody-receptor constructs, mH2/ gamma R2 - Ab portion
atggattttcaagtgcagattttcagcttcctgctaatcagtgcctcagtcataatatccagaggacaaattgttctcacccagtctccggcaatcatgtctgcatctccaggggagagggtcaccatgacctgcagtgccagctcaagtgtaagtcacatgcactggtaccagcagaagtcaggcacctcccccaaaagatggatttatgacacattcaagttgacttctggagtccctgatcgcttcagtggcagtgggtctgggacctcttactctctcacaatcagcaacatggaggctgaagatgttgccacttattactgccagcagtggagtagaaacccacccacgttcggtgttgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtcagcag

SEQ ID NO: 88 - DNA, 678 bp, Artificial Sequence
  Nucleotide and amino acid sequences of chimeric antibody-receptor constructs, SPd6xHis-hH2/gammaR1 - Ab portion
ccggatccccaggtgcagctggtgcagtctggggcagaggtgaaaaagcccggggagtctctgaagatctcctgtcagggttctggctacaagtttaccagttattggatcggctgggtgcgccagatgcccgggaaaggcctggagtggatggcgatcgtctatccttctgactctgatgccagatatagcccgtccttccaaggccgggtcaccatctcagccgacaagtccaccagcaccgcctacatgcagtggagcgccctgaatacctcggacaccgccatttatttctgtgcgcgaggcggctggcgtgacgcttttgatgtctggggccaagggacaattgtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaa

SEQ ID NO: 89 - DNA, 676 bp, Artificial Sequence
  Nucleotide and amino acid sequences of chimeric antibody-receptor constructs, SPFL-hL2/gammaR2 - Ab portion
tgacaaggcggatcccgtgatgacccagtctccagactccctggctgtgtctctgggcgagagggccaccatcaactgcaagtccagccagagtgttgtatccagctccgacaataggtactacttagcttggtaccagcagaaaccaggacagcctcctaagctgctcatttactgggcatctacccgggaatccggggtccctgaccgattcagtggcagcgggtctgggacagatttcactctcaccatcagcagcctacaggctgaagatatggcagtttattactgtcagcaatattatagtcctccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtcagcag

SEQ ID NO: 90 - DNA, 681 bp, Artificial Sequence
  Nucleotide and amino acid sequences of chimeric antibody-receptor constructs, SPFL-mFc/gammaR1 - Ab portion
gacaaggcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 91 - DNA, 675 bp, Artificial Sequence
  Nucleotide and amino acid sequences of chimeric antibody-receptor constructs, SPFL-mFc/IFN-lamdaR1 - Ab portion
gcggatcctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 92 - DNA, 675 bp, Artificial Sequence; identical to SEQ ID NO: 91
  Nucleotide and amino acid sequences of chimeric antibody-receptor constructs, SPFL-mFc/IFN-alphaR2 - Ab portion

SEQ ID NO: 93 - DNA, 675 bp, Artificial Sequence; identical to SEQ ID NO: 91
  Nucleotide and amino acid sequences of chimeric antibody-receptor constructs, SPFL-mFc/IL-20R1 - Ab portion

SEQ ID NO: 94 - DNA, 675 bp, Artificial Sequence; identical to SEQ ID NO: 91
  Nucleotide and amino acid sequences of chimeric antibody-receptor constructs, SPFL-mFc/IL-20R1 - Ab portion

SEQ ID NO: 95 - DNA, 2454 bp, Artificial Sequence; identical to SEQ ID NO: 65
  Nucleotide and amino acid sequences of chimeric antibody-receptor constructs, SPFL-mFc/EGFR - Ab Portion

SEQ ID NO: 96 - DNA, 663 bp, Artificial Sequence
  Nucleotide and amino acid sequences of chimeric antibody-receptor constructs, IL-10R2-2xlinker-L2-linker-FL - Ab portion
gtgatgtcacagtctccatcctccctacctgtgtcagttggcgagaaggttactttgagctgcaagtccagtcagagccttttatatagtggtaatcaaaagaactacttggcctggtaccagcagaaaccagggcagtctcctaaactgctgatttactgggcatccgctagggaatctggggtccctgatcgcttcacaggcagtggatctgggacagatttcactctctccatcagcagtgtgaagactgaagacctggcagtttattactgtcagcagtattatagctatcccctcacgttcggtgctgggaccaagctggtgctgaaacgggccgcggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttcgtcgggg

SEQ ID NO: 97 - DNA, 653 bp, Artificial Sequence
  Nucleotide and amino acid sequences of chimeric antibody-receptor constructs IL-10R1-2xlinker-H2-linker-6xHis - Ab portion
aggttcagttgcagcagtctgacgctgagttggtgaaacctggggcttcagtgaagatttcctgcaaggcttctggctacaccttcactgaccatgcaattcactgggtgaaacagaaccctgaacagggcctggaatggattggatatttttctcccggaaatgatgattttaaatacaatgagaggttcaagggcaaggccacactgactgcagacaaatcctccagcactgcctacgtgcagctcaacagcctgacatctgaggattctgcagtgtatttctgtacaagatccctgaatatggcctactggggtcaaggaacctcagtcaccgtctcctcagccaaaacgacgggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 98 - DNA, 1335 bp, Artificial Sequence
  Nucleotide and amino acid sequences of chimeric antibody-receptor constructs IL-10R1-2xlinker-H2-Fc - Ab portion
caggttcagttgcagcagtctgacgctgagttggtgaaacctggggcttcagtgaagatttcctgcaaggcttctggctacaccttcactgaccatgcaattcactgggtgaaacagaaccctgaacagggcctggaatggattggatatttttctcccggaaatgatgattttaaatacaatgagaggttcaagggcaaggccacactgactgcagacaaatcctccagcactgcctacgtgcagctcaacagcctgacatctgaggattctgcagtgtatttctgtacaagatccctgaatatggcctactggggtcaaggaacctcagtcaccgtctcctcagccaaaacgacgggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 99 - DNA, 1335 bp, Artificial Sequence; identical to SEQ ID NO: 98
  Nucleotide and amino acid sequences of chimeric antibody-receptor constructs, IFN-lamdaR1-linker-H2-Fc - Ab portion

SEQ ID NO: 100 - DNA, 1338 bp, Artificial Sequence
  Nucleotide and amino acid sequences of chimeric antibody-receptor constructs, IL-22R1-2xlinker-H2-Fc - Ab portion
caggttcagttgcagcagtctgacgctgagttggtgaaacctggggcttcagtgaagatttcctgcaaggcttctggctacaccttcactgaccatgcaattcactgggtgaaacagaaccctgaacagggcctggaatggattggatatttttctcccggaaatgatgattttaaatacaatgagaggttcaagggcaaggccacactgactgcagacaaatcctccagcactgcctacgtgcagctcaacagcctgacatctgaggattctgcagtgtatttctgtacaagatccctgaatatggcctactggggtcaaggaacctcagtcaccgtctcctcagccaaaacgacgggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 101 - DNA, 72 bp, Artificial Sequence
  Nucleotide and amino acid sequences of chimeric antibody-receptor constructs, mH2/ gamma R1 - Transmembrane
tggctagcgccagttgttgctgctttactactctttctagtgcttagcctggtattcatctgtttttatatt

SEQ ID NO: 102 - DNA, 72 bp, Artificial Sequence
  Nucleotide and amino acid sequences of chimeric antibody-receptor constructs, mH2/ gamma R2 - Transmembrane
tggctagcgatctccgtgggaacattttcgttgctgtcggtgctggcaggagcctgtttcttcctggtcctg

SEQ ID NO: 103 - DNA, 72 bp, Artificial Sequence; identical to SEQ ID NO: 101
  Nucleotide and amino acid sequences of chimeric antibody-receptor constructs, SPd6xHis-hH2/gammaR1- Transmembrane

SEQ ID NO: 104 - DNA, 72 bp, Artificial Sequence; identical to SEQ ID NO: 102
  Nucleotide and amino acid sequences of chimeric antibody-receptor constructs, SPFL-hL2/gammaR2 - Transmembrane

SEQ ID NO: 105 - DNA, 72 bp, Artificial Sequence; identical to SEQ ID NO: 101
  Nucleotide and amino acid sequences of chimeric antibody-receptor constructs, SPFL-mFc/gammaR1 - Transmembrane

SEQ ID NO: 106 - DNA, 69 bp, Artificial Sequence
  Nucleotide and amino acid sequences of chimeric antibody-receptor constructs, SPFL-mFc/IFN-lamdaR1 - Transmembrane
tggctagccctggtgctgccatcgcttctgatactgctgttagtaattgccgcagggggtgtgatctgg

SEQ ID NO: 107 - DNA, 63 bp, Artificial Sequence
  Nucleotide and amino acid sequences of chimeric antibody-receptor constructs, SPFL-mFc/IFN-alphaR2 - Transmembrane
tggctagcaataattactgtgtttttgatagcattggtcttgacaagcaccatagtgacactg

SEQ ID NO: 108 - DNA, 74 bp, Artificial Sequence
  Nucleotide and amino acid sequences of chimeric antibody-receptor constructs, SPFL-mFc/IL-20R1 - Transmembrane
tggctagcctggtatgttttgcccatatctattaccgtgtttcttttttctgtgatgggctattccatctaccg

SEQ ID NO: 109 - DNA, 69 bp, Artificial Sequence
  Nucleotide and amino acid sequences of chimeric antibody-receptor constructs, SPFL-mFc/IL-20R1 - Transmembrane
tggctagcctccggagccttcctgttctccatgggcttcctcgtcgcagtactctgctacctgagctac

SEQ ID NO: 110 - DNA, 66 bp, Artificial Sequence
  Nucleotide and amino acid sequences of chimeric antibody-receptor constructs, SPFL-mFc/EGFR, Transmembrane
tggctagcgatggtgggggccctcctcttgctgctggtggtggccctggggatcggcctcttcatg

SEQ ID NO: 111 - DNA, 657 bp, Artificial Sequence
  Nucleotide and amino acid sequences of chimeric antibody-receptor constructs, mH2/ gamma R1 - Intracellular
aagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttaaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttca

SEQ ID NO: 112 - DNA, 198 bp, Artificial Sequence
  Nucleotide and amino acid sequences of chimeric antibody-receptor constructs, mH2/ gamma R2 - Intracellular
aaatatagaggcctgattaaatactggtttcacactccaccaagcatcccattacagatagaagagtatttaaaagacccaactcagcccatcttagaggccttggacaaggacagctcaccaaaggatgacgtctgggactctgtgtccattatctcgtttccggaaaaggagcaagaagatgttctccaaacgctt

SEQ ID NO: 113 - DNA, 657 bp, Artificial Sequence
  Nucleotide and amino acid sequences of chimeric antibody-receptor constructs, SPd6xHis-hH2/gammaR1 - Intracellular
aagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttgaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttca

SEQ ID NO: 114 - DNA, 198 bp, Artificial Sequence; identical to SEQ ID NO: 112
  Nucleotide and amino acid sequences of chimeric antibody-receptor constructs, SPFL-hL2/gammaR2 - Intracellular

SEQ ID NO: 115 - DNA, 657 bp, Artificial Sequence; identical to SEQ ID NO: 111
  Nucleotide and amino acid sequences of chimeric antibody-receptor constructs, SPFL-mFc/gammaR1 - Intracellular

SEQ ID NO: 116 - DNA, 816 bp, Artificial Sequence
  Nucleotide and amino acid sequences of chimeric antibody-receptor constructs, SPFL-mFc/IFN-lamdaR1 - Intracellular
aagaccctcatggggaacccctggtttcagcgggcaaagatgccacgggccctggacttttctggacacacacaccctgtggcaacctttcagcccagcagaccagagtccgtgaatgacttgttcctctgtccccaaaaggaactgaccagaggggtcaggccgacgcctcgagtcagggccccagccacccaacagacaagatggaagaaggaccttgcagaggacgaagaggaggaggatgaggaggacacagaagatggcgtcagcttccagccctacattgaaccaccttctttcctggggcaagagcaccaggctccagggcactcggaggctggtggggtggactcagggaggcccagggctcctctggtcccaagcgaaggctcctctgcttgggattcttcagacagaagctgggccagcactgtggactcctcctgggacagggctgggtcctctggctatttggctgagaaggggccaggccaagggccgggtggggatgggcaccaagaatctctcccaccacctgaattctccaaggactcgggtttcctggaagagctcccagaagataacctctcctcctgggccacctggggcaccttaccaccggagccgaatctggtccctgggggacccccagtttctcttcagacactgaccttctgctgggaaagcagccctgaggaggaagaggaggcgagggaatcagaaattgaggacagcgatgcgggcagctggggggctgagagcacccagaggaccgaggacaggggccggacattggggcattacatggccaggtga

SEQ ID NO: 117 - DNA, 756 bp, Artificial Sequence
  Nucleotide and amino acid sequences of chimeric antibody-receptor constructs, SPFL-mFc/IFN-alphaR2 - Intracellular
aaatggattggttatatatgcttaagaaatagcctccccaaagtcttgaattttcataactttttagcctggccatttcctaacctgccaccgttggaagccatggatatggtggaggtcatttacatcaacagaaagaagaaagtgtgggattataattatgatgatgaaagtgatagcgatactgaggcagcgcccaggacaagtggcggtggctataccatgcatggactgactgtcaggcctctgggtcaggcctctgccacctctacagaatcccagttgatagacccggagtccgaggaggagcctgacctgcctgaggttgatgtggagctccccacgatgccaaaggacagccctcagcagttggaactcttgagtgggccctgtgagaggagaaagagtccactccaggacccttttcccgaagaggactacagctccacggaggggtctgggggcagaattaccttcaatgtggacttaaactctgtgtttttgagagttcttgatgacgaggacagtgacgacttagaagcccctctgatgctatcgtctcatctggaagagatggttgacccagaggatcctgataatgtgcaatcaaaccatttgctggccagcggggaagggacacagccaacctttcccagcccctcttcagagggcctgtggtccgaagatgctccatctgatcaaagtgacacttctgagtcagatgttgaccttggggatggttatataatgagatga

SEQ ID NO: 118 - DNA, 835 bp, Artificial Sequence
  Nucleotide and amino acid sequences of chimeric antibody-receptor constructs, SPFL-mFc/IL-20R1 - Intracellular
atatatccacgttggcaaagagaaacacccagcaaatttgattttgatttatggaaatgaatttgacaaaagattctttgtgcctgctgaaaaaatcgtgattaactttatcaccctcaatatctcggatgattctaaaatttctcaccaggatatgagtttactgggaaaaagcagtgatgtatccagccttaatgatcctcagcccagcgggaacctgaggccccctcaggaggaagaggaggtgaaacatttagggtatgcttcgcatttgatggaaattttttgtgactctgaagaaaacacggaaggtacttctctcacccagcaagagtccctcagcagaacaatacccccggataaaacagtcattgaatatgaatatgatgtcagaaccactgacatttgtgcggggcctgaagagcaggagctcagtttgcaggaggaggtgtccacacaaggaacattattggagtcgcaggcagcgttggcagtcttgggcccgcaaacgttacagtactcatacacccctcagctccaagacttagaccccctggcgcaggagcacacagactcggaggaggggccggaggaagagccatcgacgaccctggtcgactgggatccccaaactggcaggctgtgtattccttcgctgtccagcttcgaccaggattcagagggctgcgagccttctgagggggatgggctcggagaggagggtcttctatctagactctatgaggagccggctccagacaggccaccaggagaaaatgaaacctatctcatgcaattcatggaggaatgggggttatatgtgcagatggaaaac

SEQ ID NO: 119 - DNA, 969 bp, Artificial Sequence
  Nucleotide and amino acid sequences of chimeric antibody-receptor constructs, SPFL-mFc/IL-20R1 - Intracellular
agatatgtcaccaagccgcctgcacctcccaactccctgaacgtccagcgagtcctgactttccagccgctgcgcttcatccaggagcacgtcctgatccctgtctttgacctcagcggccccagcagtctggcccagcctgtccagtactcccagatcagggtgtctggacccagggagcccgcaggagctccacagcggcatagcctgtccgagatcacctacttagggcagccagacatctccatcctccagccctccaacgtgccacctccccagatcctctccccactgtcctatgccccaaacgctgcccctgaggtcgggcccccatcctatgcacctcaggtgacccccgaagctcaattcccattctacgccccacaggccatctctaaggtccagccttcctcctatgcccctcaagccactccggacagctggcctccctcctatggggtatgcatggaaggttctggcaaagactcccccactgggacactttctagtcctaaacaccttaggcctaaaggtcagcttcagaaagagccaccagctggaagctgcatgttaggtggcctttctctgcaggaggtgacctccttggctatggaggaatcccaagaagcaaaatcattgcaccagcccctggggatttgcacagacagaacatctgacccaaatgtgctacacagtggggaggaagggacaccacagtacctaaagggccagctccccctcctctcctcagtccagatcgagggccaccccatgtccctccctttgcaacctccttccggtccatgttccccctcggaccaaggtccaagtccctggggcctgctggagtcccttgtgtgtcccaaggatgaagccaagagcccagcccctgagacctcagacctggagcagcccacagaactggattctcttttcagaggcctggccctgactgtgcagtgggagtcc

SEQ ID NO: 120 - DNA, 1626 bp, Artificial Sequence
  Nucleotide and amino acid sequences of chimeric antibody-receptor constructs, SPFL-mFc/EGFR, Intracelluar
cgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcaactcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagca

SEQ ID NO: 121 - DNA, 660 bp, Artificial Sequence
  Nucleotide and amino acid sequences of chimeric antibody-receptor constructs, IL-10R2-2xlinker-L2-linker-FL - Extracellular
atggcgtggagccttgggagctggctgggtggctgcctgctggtgtcagcattgggaatggtgccacctcccgaaaatgtcagaatgaattctgttaatttcaagaacattctacagtgggagtcacctgcttttgccgaagggaacctgactttcacagctcagtacctaagttataggatattccaagataaatgcatgaatactaccttgacggaatgtgatttctcaagtctttccaagtatggtgaccacaccttgagagtcagggctgaatttgcagatgagcattcagactgggtaaacatcaccttctgtcctgtggatgacaccattattggaccccctggaatgcaagtagaagtacttgctgattctttacatatgcgtttcttagcccctaaaattgagaatgaatacgaaacttggactatgaagaatgtgtataactcatggacttataatgtgcaatactggaaaaacggtactgatgaaaagtttcaaattactccccagtatgactttgaggtcctcagaaacctggagccatggacaacttattgtgttcaagttcgagggtttcttcctgatcggaacaaagctggggaatggagtgagcctgtctgtgagcaaacaacccatgacgaaacggtcccctcc

SEQ ID NO: 122 - DNA, 705 bp, Artificial Sequence
  Nucleotide and amino acid sequences of chimeric antibody-receptor constructs IL-10R1-2xlinker-H2-linker-6xHis - Extracellular
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcgaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaac

SEQ ID NO: 123 - DNA, 705 bp, Artificial Sequence; identical to SEQ ID NO: 122
  Nucleotide and amino acid sequences of chimeric antibody-receptor constructs IL-10R1-2xlinker-H2-Fc - Extracellular

SEQ ID NO: 124 - DNA, 678 bp, Artificial Sequence
  Nucleotide and amino acid sequences of chimeric antibody-receptor constructs, IFN-lamdaR1-linker-H2-Fc - Extracellular
atggcggggcccgagcgctggggccccctgctcctgtgcctgctgcaggccgctccagggaggccccgtctggcccctccccagaatgtgacgctgctctcccagaacttcagcgtgtacctgacatggctcccagggcttggcaacccccaggatgtgacctattttgtggcctatcagagctctcctacccgtagacggtggcgcgaagtggaagagtgtgcgggaaccaaggagctgctatgttctatgatgtgcctgaagaaacaggacctgtacaacaagttcaagggacgcgtgcggacggtttctcccagctccaagtccccctgggtggagtccgaatacctggattacctttttgaagtggagccggccccacctgtcctggtgctcacccagacggaggagatcctgagtgccaatgccacgtaccagctgcccccctgcatgcccccactggatctgaagtatgaggtggcattctggaaggagggggccggaaacaagaccctatttccagtcactccccatggccagccagtccagatcactctccagccagctgccagcgaacaccactgcctcagtgccagaaccatctacacgttcagtgtcccgaaatacagcaagttctctaagcccacctgcttcttgctggaggtcccagaagccaac

SEQ ID NO: 125 - DNA, 684 bp, Artificial Sequence
  Nucleotide and amino acid sequences of chimeric antibody-receptor constructs, IL-22R1-2xlinker-H2-Fc - Extracellular
atgaggacgctgctgaccatcttgactgtgggatccctggctgctcacgcccctgaggacccctcggatctgctccagcacgtgaaattccagtccagcaactttgaaaacatcctgacgtgggacagcgggccagagggcaccccagacacggtctacagcatcgagtataagacgtacggagagagggactgggtggcaaagaagggctgtcagcggatcacccggaagtcctgcaacctgacggtggagacgggcaacctcacggagctctactatgccagggtcaccgctgtcagtgcgggaggccggtcagccaccaagatgactgacaggttcagctctctgcagcacactaccctcaagccacctgatgtgacctgtatctccaaagtgagatcgattcagatgattgttcatcctacccccacgccaatccgtgcaggcgatggccaccggctaaccctggaagacatcttccatgacctgttctaccacttagagctccaggtcaaccgcacctaccaaatgcaccttggagggaagcagagagaatatgagttcttcggcctgacccctgacacagagttccttggcaccatcatgatttgcgttcccacctgggccaaggagagtgccccctacatgtgccgagtgaagacactgccagaccggacatggacc